(19) United States
(12) Patent Application Publication
(10) Pub. No.: US 2023/0117670 A1
(43) Pub. Date: Apr. 20, 2023

(54) BIOACTIVE SUBSTANCE COMPOSITION, SERUM-FREE MEDIUM COMPRISING THE COMPOSITION, AND USES THEREOF

(71) Applicant: Zhejiang University, Hangzhou (CN)

(72) Inventors: Xiao CHEN, Hangzhou (CN); Hong ZHANG, Hangzhou (CN); Hongwei OUYANG, Hangzhou (CN); Zi YIN, Hangzhou (CN); Weiliang SHEN, Hangzhou (CN)

(21) Appl. No.: 18/066,266

(22) Filed: Dec. 14, 2022

Related U.S. Application Data
(63) Continuation of application No. PCT/CN2021/099929, filed on Jun. 14, 2021.

(30) Foreign Application Priority Data
Jun. 14, 2020 (CN) 202010539537.0

(51) Int. Cl. C12N 5/00; C12N 5/0775; C12N 5/077

(52) U.S. Cl. CPC C12N 5/0037; C12N 5/0662; C12N 5/066; C12N 5/0667; C12N 2501/115; C12N 2501/135; C12N 2501/39; C12N 2501/91; C12N 2500/24; C12N 2501/33; C12N 2501/392; C12N 2500/46; C12N 2500/38; C12N 2501/999; C12N 2500/90; C12N 2500/32

(57) ABSTRACT

The invention provides a bioactive substance composition, a serum-free medium comprising the composition and the uses thereof. The bioactive substance composition is used for serum-free medium and/or composition and the preparation thereof; The serum-free medium and/or composition can be used for primary culture and secondary culture of cells and/or tissues. The cells are selected from any one or more of tendon and/or ligament derived cells, chondrocytes, meniscus stem cells, mesenchymal stem cells, skeleton stem cells, and muscle stem cells. The tissue is the musculoskeletal system tissue. The bioactive substance composition and/or serum-free medium and/or the composition can be used to prepare drugs for tissue and/or organ injury treatment; The tissue or organ injury is selected from the tissue or organ injury of the musculoskeletal system.

### CROSS REFERENCE TO RELATED APPLICATIONS

The present application is a continuation of International Patent Application No. PCT/CN2021/099929, entitled “BIOACTIVE SUBSTANCE COMPOSITION, SERUM-FREE MEDIUM COMPRISING THE COMPOSITION, AND USES THEREOF,” filed Jun. 14, 2021, which claims priority to Chinese Patent Application No. 202010539537.0, entitled the same and filed Jun. 14, 2020 in the China National Intellectual Property Administration (CNIPA), the entire of which is hereby incorporated in its entirety by reference.

### TECHNICAL FIELD

This invention belongs to the technical field of biomedicine, and specifically relates to preparation of a serum-free medium containing bioactive substances and its application.

### BACKGROUND

Data from the World Health Organization shows that tissue and organ injury is an severe health problem, which is the second leading cause of disability. Due to the rapid development of industry, agriculture, transportation and sports, the trauma caused by various accidents is increasing. In 2008, the data released by the National Bureau of Statistics showed that trauma and poisoning were the fifth leading cause of death. With the development of social economy and the acceleration of transportation and urban construction, the incidence of tissue injury has increased significantly, which mainly are caused by sports injuries, traffic accidents, natural disasters (such as the Wenchuan and Yushu earthquakes), and industrial accidents (such as the explosion accident in Tianjin Port, the explosion of the PX factory in Zhangzhou, Fujian), etc. And because the population of severe trauma is mainly young and middle-aged, the loss of social productivity appears to be particularly serious. Meanwhile, along with aging, the regeneration and repair ability of human tissues and organs declines, leading to the occurrence of various diseases. Therefore, how to repair injured tissues quickly and effectively has become a major health problem that restricts social and economic development that needs to be resolved urgently.

In recent years, the development of cell therapy, tissue engineering technology and molecular biology technology has brought new opportunities to improve the quality of tissue repair. Researchers have begun to try to use tissue engineering technology and cell therapy to treat and repair tissue defects. Obtaining ideal seed cells is the key to tissue repair and regeneration. Common cells used for cell therapy include mesenchymal stem cells (MSCs), adipose-derived stem cells (ADSCs, or Adipose stem cells, ASCs), tendon and/or ligament-derived cells and chondrocytes, etc. However, the number of cells naturally present in vivo that can be used for cell therapy is small and cannot meet the needs of cell therapy. Therefore, in vitro expansion of stem cells with good quality and quantity is the guarantee for the current implementation of stem cell therapy.

The natural microenvironment for cell growth in the body is very complex. Except for vascular wall cells and blood-related cells, all other cells in the body are in the extracellular fluid without serum. Complex extracellular fluid, cell interaction through the extracellular matrix and cell-cell interactions constitute a complex natural microenvironment for cell growth in vivo, which constitute the complex natural microenvironment for cell growth in the body. Cells from different tissues and organs are in different microenvironment in the body, such as different types and contents of extracellular fluid, different interactions among cells, and different interactions between cells and extracellular matrix. These microenvironments match the functions of tissues or organs and can well promote cell proliferation, phenotype maintenance, metabolism and other life activities in the body to maintain tissue/organ function. At present, we know very little about the specific components and interaction mechanisms of the complex and diverse microenvironment in the body. We cannot be well inspired to simulate the microenvironment in vivo for cell culture in vitro. Nowadays, the culture environment of stem cells in vitro is mainly culture medium containing serum. The commonly used serum is fetal bovine serum. The extracellular fluid microenvironment in which the cells in the body are located is serum-free, and due to the complex extracellular fluid, cell-cell interaction, and cell-extracellular matrix interaction, the growth environment of the cells in the body is particularly complex, making the difference between the in vivo and in vitro environments very large. Moreover, the in vitro stem cell culture environment cannot maintain the proliferation and phenotype of the cultured cells. Therefore, how to better simulate the growth environment of in vivo cells in vitro is a major problem that has not yet been resolved in in vitro cell culture.

Taking the in vitro culture of cells derived from tendons and/or ligaments as an example:

Tendon and/or ligament-derived cells are a mixture of cells isolated from tendon or ligament tissue. They are highly expressing multiple tendon/ligament tissue-specific genes and proteins, including scleraxis (SCX), nestin (NES), tenomodulin (TNMD), thrombospondin-4 (THBS4), collagen type I alpha 1 (COL1, COL1A1), Tenascin-C (TNC), etc. These cells contain tendon stem/progenitor cells (TSPCs), tenocytes, tenoblasts, fibroblasts, ligament stem/progenitor cells, ligament cells, etc, which are the most ideal seed cells for tendon injury treatment. Researchers in the field at home and abroad have successively isolated tendon stem cells from mouse, human, rat, and rabbit tendons, and made comprehensive identifications of their functions and phenotypes. Studies have shown that these cells not only have stem cell features like bone marrow mesenchymal stem cells, but also highly express multiple tendon tissue-specific genes and proteins, including SCX, NES, TNMD, THBS4, COL1, Tenascin-C. Therefore, cells derived from tendons and/or ligaments, especially tendon stem cells, are considered suitable seed cells for tendon tissue engineering and tendon injury cell therapy.

Due to the small number of cells present in mature tendon and ligament tissues, the number of extracted tendon or ligament-derived cells is not enough to be used for tendon regeneration. It is necessary to expand these cells in vitro culture environment to obtain cells derived from tendons and/or ligaments with good quantity and quality. Therefore, in order to meet the purpose of clinical treatment of tendon and/or ligament injury, cells derived from tendon and/or ligament cultured in vitro must meet certain quality and quantity requirements at the same time. Cells derived from tendons and/or ligaments not only share the features of mesenchymal stem cells, adipose stem cells and other stem cells, but also have their unique tendon phenotypes. The quality and quantity requirements mentioned above are mainly evaluated and scored from two major aspects: the common features of stem cells and the unique phenotype of tendon/ligament-derived cells. The score is 100 points. The specific scoring criteria are as follows (Bi Y et al., Nature medicine, 2007,13(10):1219-27. Harvey T, Nature cell biology, 2019,21(12):1490-1503. Yin Z et al., Science advances, 2016,2(11):e1600874. Lee S Y, Stem Cells, 2015,33(10):2995-3005. Zhang C, Biomaterials, 2018,172:66-82.):

Common Features of Stem Cells (Also Called Common Features of Cells, Cell Common Features) (60 Points)

1. Proliferation rate (30 points): If the cell doubling time is within 30 h, the score is 30 points. Cell doubling time within 76 h or proliferation rates more than 5 folds a week is considered as a passing line for 10 points. Cell doubling time between 76 h-100 h is scored as 5 points, and cell doubling time above 100 h is scored as 0 points.

2. Stem cell phenotype (20 points): The phenotype of stem cells includes the surface markers of stem cells, clone formation ability, and three-lineage differentiation ability. Stem cell surface markers include positive markers CD105, CD90, CD44, whose expression must be above 95% as phenotypic maintenance or improvement, and negative markers CD34, CD18, whose expression must be 1% or less as phenotype maintenance. Clone forming ability refers to the ability of a cell to form a single clone. Cells whose clonal formation ability is better than those cultivated by the existing serum culture technology are considered to have improved clonal formation ability, and the maintenance refers to the clone formation ability is consistent with the cells cultured by the existing serum culture technology. The three-lineage differentiation ability includes osteogenic differentiation, chondrogenic differentiation and adipogenic differentiation. Cells whose three-line differentiation ability is better than cells cultured with the existing serum culture technology are considered to have improved three-lineage differentiation ability, and the three-line differentiation ability of cells that are consistent with the cells cultured with the existing serum culture technology is considered to have maintained three-lineage differentiation ability. Stem cell surface markers, cloning ability and three-lineage differentiation ability are all increased as 20 points, one or two items are improved and other maintenance is regarded as 15 points, three items are maintained as 10 points, two items are reduced as 5 points, and three items declines are considered as 0 points. Other scores are based on the situation.

3. Safety (10 points): This characteristic includes normal karyotype, no serum residue, and no viral Mycoplasma contamination. Karyotype analysis results show that more than 90% of the cell karyotype is consistent with the normal karyotype of the species, and the cultured cell is considered to be normal. All three items that have achieved safety will be rated as a passing line 10 points. If any of the three items fails to achieve safety, the score is 0. As long as the cells have been cultured with serum, whether they are primary culture or secondary culture, they will be deemed to have residual serum, and the score is 0; only the primary culture and secondary culture do not use serum can they be considered as free of serum.

Cell-Specific Phenotypes Derived from Tendons and/or Ligaments (Also Called Cell-Specific Phenotypes) (40 Points)

4. Tendon phenotype and tenogenic differentiation ability (20 points): This item involves SCX, Nestin, TNMD, THBS4, COL1 and other tendon gene or protein markers, collagen forming ability. If the cells express three or more tendon genes or protein markers such as SCX, Nestin, TNMD, THBS4, COL1, and the positive rate of tendon markers such as Nestin is more than 90%, the score is 20 points; If the cells express three or more tendon genes or protein markers such as SCX, Nestin, TNMD, THBS4, COL1, and the positive rate of tendon markers such as Nestin is more than 60%, the score is 15 points; If the cells express two tendon genes or protein markers such as SCX, Nestin, TNMD, THBS4, COL1, and the positive rate of tendon markers such as Nestin is more than 30%, the score is 10 points; If the cells express one tendon genes or protein markers such as SCX, Nestin, TNMD, THBS4, COL1, and the positive rate of tendon markers such as Nestin is more than 10%, the score is 5 points (pass line); If the cells don't express or underexpress tendon genes or protein markers such as SCX, Nestin, TNMD, THBS4, COL1, and the positive rate of tendon markers such as Nestin is less than 10%, the score is 0 points (pass line). Other scores are based on the situation. Among them, “high expression” refers to cells cultured with existing serum culture technology as a control, and the expression of genes or protein markers in cultured cells is relatively higher than that of the control; “Low expression” means that the gene or protein marker expression of the cultured cells is relatively lower than that of the control; “No expression” means that the expression of the cultured cell gene or protein marker is 0 or the gene or protein marker expression level is extremely low so that it cannot be detected.

5. Tendons and/or ligaments repair ability in vivo (20 points): If the histological morphology of the repair tissue is close to normal tissue, the collagen is dense, neatly arranged, and no formation of non-tendon and/or ligament tissues such as bone, cartilage, muscle, etc., it will be scored as 20 points. If there is a small defect in the repaired tissue, the collagen formation and arrangement are relatively neat, and there is no bone, cartilage and other non-tendon tissues in the repaired tissue, it is scored as a passing line of 10 points. If there is large defect or non-tendon tissue such as bone, cartilage and muscle formation in the repaired tissue, the score is 0 points. Specific scores are based on the degree of the tissue repair.

In vitro cultured tendon and/or ligament-derived cells must meet the requirements for clinical treatment of tendon and/or ligament injury, that is, the five individual items contained in the common features of the cells and the cell-specific phenotype have reached their respective passing lines and the total cell score 60 points or more, which means that the cell indicators are qualified and can meet the requirements of clinical cell therapy. The higher the score, the better the quality and quantity of cultured cells, and the more it meets the needs of clinical cell therapy. That is, each of the five individual indicators of proliferation rate, stem cell phenotype, safety, tendon phenotype and tenogenic differentiation ability, as well as tendon and/or ligament repair ability in vivo has reached its own passing line and the total score is greater than or equal to 60 points can be considered qualified. If any one of the five individual indicators fails to reach the passing line or the total cell score is less than 60 points, the cell indicators are deemed to be unqualified and not suitable for clinical cell therapy.

In vitro cell culture refers to a method of simulating the complex environment of in vivo cell growth in vitro to enable it to survive, grow, reproduce, and maintain its main structure and function. In vitro cell culture mainly includes the following two steps: 1) Primary culture: refers to the first culture of tissues or cells removed from the body. Strictly speaking, the tissues or cells are removed from the body and cultured to the first passage. The cells cultured in this stage are called primary cells (P0). This process involves the conversion of cells from the in vivo microenvironment to the in vitro culture environment. In order to make the cells adapt to the in vitro environment for proliferation as soon as possible, the in vitro culture environment of primary cells needs to mimic the complex microenvironment in the body as much as possible, and the environmental requirements are higher than that of secondary culture. 2) Secondary culture: The cells cultured in the primary (P0) or other generations of cells that have been cultured in vitro are separated into single cells for secondary culture. Tendon and/or ligament-derived cells are generally cultured to the fifth generation (P5) or higher. According to the common features of stem cells and the maintenance of the unique phenotype of tendon and/or ligament-derived cells, suitable generation cells are selected for tendon injury repair or other purposes.

The culture medium is a key factor for cell expansion in vitro. The medium refers to a soluble liquid nutrient matrix prepared by a combination of different nutrients for cell growth and reproduction. The culture medium is not only the basic material for providing nutrition and promoting cell proliferation to the cells cultured in vitro, but also the in vitro living environment for cell growth and reproduction. It can realize cell primary culture and secondary culture.

Because the microenvironment in vivo of cells is very complicated, there are still many components/factors that have not been studied clearly. Animal serum/plasma, such as fetal bovine serum, has a wide range of sources and mature preparation technology, which contains a wealth of nutrients such as protein, hormones, enzymes, etc., and can promote the growth of cells. Therefore, in the current in vitro culture of tendon and/or ligament-derived cells, especially primary culture, in order to make the cells adapt to the in vitro environment for proliferation as quickly as possible, the in vitro culture environment of primary cells needs to mimic the complex microenvironment in the body as much as possible. The environment requirement is higher than that of secondary culture. Only serum/plasma-containing medium can be used to provide cells with an environment that is relatively similar to that of living organisms. However, studies have found that the tendon and/or ligament-derived cells cultured in vitro in serum-containing media cannot simultaneously meet the quality and quantity requirements for the clinical treatment of tendon and/or ligament injuries. The total cell score is below 60, that is, the cell index is not qualified, the specific reasons are as follows: 1) Cells derived from tendons and/or ligaments in in vitro serum-containing media proliferate slowly, and replicative senescence is prone to occur with the increase in the number of cultures in vitro, resulting in slower and slower cell proliferation, which cannot be expanded for sufficient cell mass in a short period of time; 2) Serum-cultured tendon and/or ligament-derived cells cannot maintain the tendon phenotype as the number of cultures increases. This is mainly manifested in the gradual decline or complete loss of tendon gene or protein expression such as SCX, DCN, TNMD, etc. (FIG. 1-2), high expression of bone line genes alkaline phosphatase (ALP), osteocalcin (OCN), etc.; 3) Animal experiments show that the repaired tendon and/or ligament with serum-cultured tendon and/or ligament-derived cells are composed of large number of small-diameter collagen fibers, and their function is significantly lower than that of normal tendons (FIG. 3). Not only that, the tendon and/or ligament-derived cells obtained under serum-containing culture are prone to heterotopic ossification when used for tendon and/or ligament repair, that is, bone tissue grows out of the tendon and/or ligament tissue, resulting repair failure of tendon and/or ligament tissue (FIG. 4); 4) The complexity and characteristics of the serum itself lead to safety hazards in the cells derived from tendons and/or ligaments obtained by its culture, including: a. The use of serum has the risk of contaminating foreign viruses and pathogenic factors, and it is easy to cause the cultured cells to be contaminated by viruses and Mycoplasma; b. The composition of serum is complex and unclear, and it is easy to remain in the cell products and cause the allergic reaction of the inoculated person to the serum, which is not conducive to animal experiments or clinical trials; c. There are batch differences in serum or plasma. Inconsistent biological activities and factors between different batches of serum will result in poor reproducibility of cell products and experimental results, which will require a lot of verification work. Therefore, it is necessary to explore suitable methods to replace the existing serum culture technology, reduce or avoid the adverse effects of serum, and expand the tendon and/or ligament-derived cells with a total cell score greater than or equal to 60 points for clinical tendon and/or ligament injury.

Existing research attempts to use single or multiple physical factors to reduce or avoid the adverse effects of serum culture, as far as possible to meet the quality and quantity requirements of the cells used in the clinical treatment of tendon and/or ligament injuries. The physical factors mainly include the topological structure of the bottom surface of the culture medium, hardness and mechanical stimulation. The addition of these factors can maintain the tendon phenotype of the serum-cultured tendon and/or ligament-derived cells to a certain extent, but its effect of promoting cell proliferation is average. Cell proliferation is slow, the amount of cells obtained is small, and it is impossible to guarantee a sufficient number of cells at the same time. Moreover, the addition of physical factors still needs to be based on serum culture, which cannot avoid the potential safety hazards caused by serum itself, nor can it replace serum culture. It also increases the complexity of the culture system and increases the difficulty of implementation. Therefore, this culture method cannot meet the quality and quantity requirements of clinical treatment cells for tendon and/or ligament injuries at the same time, and the total score of cultured cells is lower than the pass line.

Serum-free medium or serum-free culture medium refers to a liquid nutrient matrix that does not contain blood-derived substances such as serum, plasma, platelet-rich plasma (PRP), and contains a variety of well-defined biologically active substances, inorganic salts, and water. Serum-free medium is divided into completely serum-free medium and partial serum-free medium. Completely serum-free medium refers to the ability to achieve serum-free primary culture of cells in vitro, as well as support the serum-free secondary culture of cells in vitro. It provides nutrients required for life activities such as cell proliferation, phenotype maintenance, metabolism, etc. The partial serum-free medium refers to a serum-free medium that cannot be completely separated from blood-derived substances such as serum for cell culture, and can only support the secondary culture of cells, but cannot support the primary culture of cells. Since cells are separated from the complex and suitable microenvironment in the body during the primary culture and converted to the in vitro culture environment, there will be an adaptation process. The closer the in vitro culture environment is to the complex growth microenvironment of the cells in the body, the shorter the cell adaptation process. Therefore, the process of primary culture needs to provide cells with a more bionic environment than secondary culture so that the cells can rejuvenate as soon as possible to adapt to the in vitro culture environment for expansion.

The serum-free medium of prior technology for tendon and/or ligament-derived cells cannot replace the role of serum for primary culture, and can only achieve the secondary culture of tendon and/or ligament-derived cells, so they all belong to partial serum-free medium. The cultured cells cannot meet the requirements of clinical cell therapy. Specifically, in the prior art, single or multiple growth factors or cytokines are combined with basic media such as DMEM or F12 to form part of a serum-free medium, which cannot replace serum for primary culture of tendon and/or ligament-derived cells. The cultured cells are still derived from the existing serum culture technology, which brings the adverse effects of the existing serum culture technology. Subsequent secondary culture with these partial serum-free media can only relieve to a certain extent but cannot avoid the adverse effects of serum culture. Moreover, the secondary culture effect of these partial serum-free media is not good, and the total score of cultured cells is lower than the pass line, which cannot meet the requirements of the number and quality of cells required for clinical treatment of tendon and/or ligament injuries (Cells Tissues Organs 2013; 197:27-36, CHINESE JOURNAL OF SURGERY. 2014. 31(2): 395-398, J. Hand Surg 2005; 30:441-447, Biomaterials 2015; 69: 99-109). Therefore, the partial serum-free medium in the prior art cannot solve the disadvantages of the existing serum culture technology, and the completely serum-free medium for tendon and/or ligament-derived cells has not yet seen research and application.

Existing commercial serum-free medium is developed for MSC, adipose stem cells (ADSC or ASC), pluripotent stem cells (PSC), neural stem cells (NSC) and other cells. The common ones are StemProTMMSC SFM XenoFree (Invitrogen, Gibco), MesenCult™-ACF Plus Medium (STEMCELL Technologies), Mesenchymal Stem Cell Growth Medium DXF (PromoCell), MSC XF (Biological Industries), StemPro™ NSC SFM (Invitrogen, Gibco), etc. Cells derived from tendons and/or ligaments are different from these stem cells and have their own features, namely, high expression of SCX, NES, TNMD, THBS4, COL1 and other tendon and/or ligament tissue-specific genes and proteins. Other cells, such as nerve cells, highly express PSA-NSAM, p75NTR, Musashi1, ASH1, CD133, GFAP and other markers that are not expressed or underexpressed by cells derived from tendons and/or ligaments. Therefore, at present, there is no relevant research and application on the completely serum-free medium developed for cells derived from tendons and/or ligaments that can replace serum for primary culture and secondary culture to meet the requirements for the quantity and quality of clinical treatment cells.

The paper (Cells Tissues Organs 2013; 197:27-36) discloses that 50 ng/mL insulin-like growth factor 1 and 10 ng/mL transforming growth factor R 3 can maintain the phenotype of tenocytes without serum. However, in the experimental methods and materials of this research, the method of separation and culture of tenocytes partly shows that the cells used in this research are primary cultured with a medium containing 20% FBS, so the medium used in this research is only partially serum-free. Basically, there is no way to avoid the disadvantages of existing serum culture technology, and the safety score of cultured cells is 0. From Comparative Example 1, it can be seen that the total score of cells cultured by serum culture technique is less than 60 points, and the paper shows that the proliferation effect of tenocytes under this condition is only ⅓ of that of the serum culture group (FIG. 5), and the formation of collagen is only ½ (FIG. 6). The expression of tendon line-specific markers such as SCX of these cells is also much lower than that of serum culture medium. Therefore, the total score of cells cultured in this way is lower than the total score of cells cultured by serum culture technology, which is much lower than 60. Not only can it not replace serum for the isolation and culture of tendon-derived cells in vitro, it also cannot meet the cell requirements for clinical treatment of tendon injuries.

The paper (Chinese Journal of Experimental Surgery. 2014. 31(2): 395-398) discloses that adding 50 μg/L IGF-1+10 μg/L TGF-β3 to α-MEM medium without serum can maintain the phenotype of human tendons. The production of collagen fibers is similar to that of tenocytes cultured with 10% FBS, and it also up-regulates the phenotype of tenocytes and the mRNA expression of differentiation markers. However, this kind of culture method cannot promote cell proliferation. The single item score of cell proliferation is 0, and the cultured cells cannot meet the cell mass requirements for clinical cell therapy.

The paper (J. Hand Surg 2005; 30:441-447) discloses that the basal medium DMEM supplemented with platelet-derived growth factor BB (PDGF-BB) and basic fibroblast growth factor (bFGF) can promote tenocyte proliferation and collagen form. In the research materials and methods, the isolation and culture of tendon fibroblasts showed that the cells used in the research were primary cultured with 10% FBS. Therefore, the medium used in this study is a partial serum-free medium, which cannot avoid the shortcomings of the existing serum culture technology, and the safety score is 0. And the results of this paper show that the cell proliferation is slow under this kind of culture conditions, and the amount of cells obtained is small, which is not enough to provide a suitable environment for cell expansion in vitro. Therefore, the total cells obtained in this article are lower than the passing line, and this result does not indicate that the addition of these two growth factors can replace the existing serum culture technology for in vitro culture of tenocytes to obtain tenocytes that meet the number of clinical cell treatments and treatment requirements.

The paper (Biomaterials 2015; 69: 99-109) discloses that biomimetic micro-tissue spheres and specific growth factor supplements are used in vitro to improve tendon cell differentiation. This culture method uses cell hanging drop technology combined with a low serum growth medium containing L-ascorbate 2-phosphate, insulin and transforming growth factor (TGF)-1 to maintain the tendon lineage phenotype of differentiated tendon cells in vitro. However, the research literature shows that its culture system still requires the participation of low-concentration serum, and the adverse effects of serum cannot be avoided. The safety score is 0, and the cell proliferation problem has not been solved, which cannot meet the needs of clinical cell therapy and quantity requirements.

For the culture of tendon and/or ligament-derived cells, although the prior researches attempt to use single or compound physical or biological factors to avoid the current drawbacks of culturing tendon and/or ligament-derived cells with serum, these techniques still require serum to participate in primary culture and even secondary culture of cells. Moreover, the effect is not good. The cells derived from tendons and/or ligaments cultured by the existing culture technology cannot simultaneously meet the quality and quantity requirements of clinical treatment cells for tendon and/or ligament injuries. The total score of the cultured cells is less than 60 points, and the cell indicators are not qualified and suitable for clinical cell therapy. However, the current commercial serum-free medium cannot maintain the specificity of cells derived from tendons and/or ligaments, and is not suitable for in vitro culture of cells derived from tendons and/or ligaments. Therefore, for tendon and/or ligament-derived cells, developing a completely serum-free medium that is conducive to the efficient proliferation and phenotype maintenance of tendon and/or ligament-derived cells, avoid the disadvantages of the existing tendon and/or ligament-derived cell culture technology, and to meet the requirements of the number and quality of cells required for clinical treatment of tendon and/or ligament injuries, is currently an urgent problem to be solved. However, the prior art does not have a corresponding solution.

### SUMMARY OF THE DISCLOSURE

Aiming at the problems of slow cell proliferation, small number, easy loss of phenotype, unstable cell quality, poor safety, and failure to repair injury after transplantation in the existing in vitro cell culture, the present invention provides a serum-free medium containing a bioactive substance composition. Surprisingly, through continuous research, the inventor invented a completely serum-free medium with defined components, which can achieve complete serum-free primary culture and secondary culture of cells. In particular, the inventor overcomes the technical prejudice that serum must be involved in the primary culture of cells derived from tendons and/or ligaments in the prior technology, and can simultaneously meet the requirements of the number and quality of cells required for clinical treatment of tendon and/or ligament injuries. The cell common phenotype and cell-specific phenotype of the cells cultured in the serum-free medium of the present invention can be maintained or improved. That is, these five items have reached their respective passing lines and the total cell score is greater than or equal to 60 points, and under the best conditions, the total cell score can reach 100 points, which can simultaneously meet the treatment and quantity requirements of clinical cell therapy.

The technical scheme of the present invention is as follows:

The first object of the present invention is to provide a bioactive substance composition comprising fibroblast growth factor, platelet-derived growth factor, transforming growth factor-β, glucocorticoid, heparin or its salt, vitamin C or its derivatives, transferrin, insulin, progesterone, putrescine or its salt, selenite.

Among them, the mass-volume concentration range ratio of each component is: Fibroblast growth factor:platelet-derived growth factor:transforming growth factor-β:glucocorticoid:heparin or its salt:vitamin C or its derivative:transferrin:insulin:progesterone:putrescine or its salt:selenite=1-50:1-50:1-40:1-11:10-5000:10-100000:10-300000:1-25000:1-25:1-25000:1-25. Preferably, fibroblast growth factor:platelet-derived growth factor:transforming growth factor-β:glucocorticoid:heparin or its salt:vitamin C or its derivative:transferrin:insulin:progesterone:putrescine or its salt:selenite=5-40:5-40:2-30:1-8:500-4000:1000-90000:1000-200000:10-15000:1-15:2-15000:1-15. More preferably, fibroblast growth factor:platelet-derived growth factor:transforming growth factor-β:glucocorticoid:heparin or its salt:vitamin C or its derivative:transferrin:insulin:progesterone:putrescine or its salt:selenite=10-30:10-30:3-20:2-5:1000-2000:10000-80000:2000-80000:100-5000:2-7:7-10000:2-7.

Further, in the bioactive substance composition, the fibroblast growth factor is selected from any one or more of FGF-basic, FGF1, FGF2, FGF4, FGF7, FGF12, FGF18, and fibroblast growth factor synthetic peptides. Preferably, the mass-volume concentration of the fibroblast growth factor in the bioactive substance composition is 1-100 ng/ml, and the mass ratio is 0.0000001%-0.00001%. Preferably, the mass-volume concentration of the fibroblast growth factor in the bioactive substance composition is 5-70 ng/ml, and the mass ratio is 0.0000005%-0.000007%. More preferably, the mass-volume concentration of the fibroblast growth factor in the bioactive substance composition is 10-40 ng/ml, and the mass ratio is 0.000001%-0.000004%.

Further, in the bioactive substance composition, the platelet-derived growth factor is selected from any one or more of PDGF-AA, PDGF-AB, PDGF-BB, and synthetic peptides of platelet-derived growth factor. Preferably, the mass-volume concentration of the platelet-derived factor in the bioactive substance composition is 1-100 ng/ml, accounting for 0.0000001%-0.00001% by mass. Preferably, the mass-volume concentration of the platelet-derived factor in the bioactive substance composition is 5-70 ng/ml, accounting for 0.0000005%-0.000007% by mass. More preferably, the mass-volume concentration of the platelet-derived factor in the bioactive substance composition is 10-40 ng/ml, accounting for 0.000001%-0.000004% by mass.

Further, in the bioactive substance composition, the transforming growth factor-β is selected from any one or more of TGF-β1, TGF-β2, TGF-β3, and transforming growth factor-β synthetic peptide. Preferably, the mass-volume concentration of the transforming growth factor-β in the bioactive substance composition is 0.1-80 ng/ml, accounting for 0.00000001%-0.000008% by mass. Preferably, the mass-volume concentration of the transforming growth factor-β in the bioactive substance composition is 2-50 ng/ml, accounting for 0.0000002%-0.000005% by mass. More preferably, the mass-volume concentration of the transforming growth factor-β in the bioactive substance composition is 5-25 ng/ml, accounting for 0.0000005%-0.0000025% by mass.

Further, in the bioactive substance composition, the glucocorticoid is selected from any one or more of dexamethasone or its salt, dexamethasone solvent, hydrocortisone or its salt, cortisone acetate, cortisone acetate or its salt, methylprednisone sodium succinate, prednisone, betamethasone, betamethasone valerate, beclomethasone propionate, prednisolone acetate, prednisolone acetate, or prednisolone. Preferably, the molar concentration of the glucocorticoid in the bioactive substance composition is 0.1-90 nM, accounting for 0.0000000039%-0.00000354% by mass. Preferably, the molar concentration of the glucocorticoid in the bioactive substance composition is 1-50 nM, accounting for 0.000000039%-0.00000197% by mass. More preferably, the molar concentration of the glucocorticoid in the bioactive substance composition is 1-20 nM, accounting for 0.000000039%-0.000000785% by mass.

Further, in the bioactive substance composition, the heparin or its salt is selected from any one or more of heparin, heparin sodium and heparin calcium. Preferably, the mass volume concentration of the heparin or its salt in the bioactive substance composition is 0.1-10 μg/ml, accounting for 0.00001%-0.001% by mass. Preferably, the mass volume concentration of the heparin or its salt in the bioactive substance composition is 0.5-8 μg/ml, accounting for 0.00005%-0.0008% by mass. More preferably, the mass volume concentration of the heparin or its salt in the bioactive substance composition is 1-5 μg/ml, accounting for 0.0001%-0.0005% by mass.

Further, in the bioactive substance composition, the vitamin C or its derivatives are selected from any one or more of Vitamin C (i.e. ascorbic acid), ascorbic acid glucoside, ethyl vitamin C, 3-o-ethyl ascorbic acid, magnesium phosphate of vitamin C, sodium phosphate of vitamin C, L-ascorbic acid 2-phosphate sesquimagnesium salt complex, vitamin C tetraisopalmitate, ascorbic acid palmitate, ascorbic acid 2-phosphate 6-palmitate, esterified vitamin C, other solvates of ascorbic acid. Preferably, the mass volume concentration of the vitamin C or its derivatives in the bioactive substance composition is 0.1-100 μg/ml, accounting for 0.00001%-0.01% by mass. Preferably, the mass volume concentration of the vitamin C or its derivatives in the bioactive substance composition is 1-100 μg/ml, accounting for 0.0001%-0.01% by mass. More preferably, the mass volume concentration of the vitamin C or its derivatives in the bioactive substance composition is 10-80 μg/ml, accounting for 0.001%-0.008% by mass.

Further, the mass-volume concentration of the transferrin in the bioactive substance composition is 0.1-300 μg/ml, and the mass ratio is 0.00001%-0.03%. Preferably, the mass-volume concentration of the transferrin in the bioactive substance composition is 1-200 μg/ml, and the mass ratio is 0.0001%-0.02%. More preferably, the mass-volume concentration of the transferrin in the bioactive substance composition is 1-150 μg/ml, and the mass ratio is 0.0001%-0.015%.

Further, the mass-volume concentration of the insulin in the bioactive substance composition is 0.01-50 μg/ml, and the mass ratio is 0.000001%-0.005%. Preferably, the mass-volume concentration of the insulin in the bioactive substance composition is 0.1-30 μg/ml, and the mass ratio is 0.00001%-0.003%. More preferably, the mass-volume concentration of the insulin in the bioactive substance composition is 1-20 μg/ml, and the mass ratio is 0.0001%-0.002%.

Further, the mass-volume concentration of the progesterone in the bioactive substance composition is 0.1-50 ng/ml, and the mass ratio is 0.00000001%-0.000005%. Preferably, the mass-volume concentration of the progesterone in the bioactive substance composition is 1-30 ng/ml, and the mass ratio is 0.0000001%-0.000003%. More preferably, the mass-volume concentration of the progesterone in the bioactive substance composition is 2-20 ng/ml, and the mass ratio is 0.0000002%-0.000002%.

Further, the putrescine or its salt is selected from any one or more of putrescine and putrescine dihydrochloride. The mass volume concentration of putrescine or its salt in the bioactive substance composition is 0.01-50 μg/ml, accounting for 0.000001%-0.005% by mass. Preferably, the mass volume concentration of putrescine or its salt in the bioactive substance composition is 0.1-40 μg/ml, accounting for 0.00001%-0.004% by mass. More preferably, the mass volume concentration of putrescine or its salt in the bioactive substance composition is 1-30 μg/ml, accounting for 0.0001%-0.003% by mass.

Further, the selenite in the bioactive substance composition is a water-soluble selenite. Preferably, the selenite is sodium selenite. The mass volume concentration of the selenite in the bioactive substance composition is 0.1-50 ng/ml, accounting for 0.00000001%-0.000005% by mass. Preferably, the mass volume concentration of the selenite in the bioactive substance composition is 1-30 ng/ml, accounting for 0.0000001%-0.000003% by mass. More preferably, the mass volume concentration of the selenite in the bioactive substance composition is 2-20 ng/ml, accounting for 0.0000002%-0.000002% by mass.

The second object of the present invention is to provide a method for preparing a bioactive substance composition, the preparation of the bioactive substance composition comprises the following steps: mixing fibroblast growth factor, platelet-derived growth factor, transforming growth factor-β, glucocorticoid, heparin or its salt, vitamin C or its derivatives, transferrin, insulin, progesterone, putrescine or its salt and selenite in proportion. The addition order of each component is not in order. The mass volume concentration range of each component is:

Fibroblast growth factor:platelet-derived growth factor:transforming growth factor-β: glucocorticoid:heparin or its salt:vitamin C or its derivative:transferrin:insulin:progesterone:putrescine or its salt:selenite=1-50:1-50:1-40:1-11:10-5000:10-100000:10-300000:1-25000:1-25:1-25000:1-25.

Preferably, fibroblast growth factor:platelet-derived growth factor:transforming growth factor-β:glucocorticoid:heparin or its salt:vitamin C or its derivative:transferrin:insulin:progesterone:putrescine or its salt:selenite=5-40:5-40:2-30:1-8:500-4000:1000-90000:1000-200000:10-15000:1-15:2-15000:1-15.

More preferably, fibroblast growth factor:platelet-derived growth factor:transforming growth factor-β:glucocorticoid:heparin or its salt:vitamin C or its derivative:transferrin:insulin:progesterone:putrescine or its salt:selenite=10-30:10-30:3-20:2-5:1000-2000:10000-80000:2000-80000:100-5000:2-7:7-10000:2-7.

Preferably, the mass-volume concentration of the transferrin in the bioactive substance composition is 0.1-300 μg/ml, and the mass ratio is 0.00001%-0.03%. Preferably, the mass-volume concentration of the transferrin in the bioactive substance composition is 1-200 μg/ml, and the mass ratio is 0.0001%-0.02%. More preferably, the mass-volume concentration of the transferrin in the bioactive substance composition is 1-150 μg/ml, and the mass ratio is 0.0001%-0.015%.

Preferably, the mass-volume concentration of the insulin in the bioactive substance composition is 0.01-50 μg/ml, and the mass ratio is 0.000001%-0.005%. Preferably, the mass-volume concentration of the insulin in the bioactive substance composition is 0.1-30 μg/ml, and the mass ratio is 0.00001%-0.003%. More preferably, the mass-volume concentration of the insulin in the bioactive substance composition is 1-20 μg/ml, and the mass ratio is 0.0001%-0.002%.

Preferably, the mass-volume concentration of the progesterone in the bioactive substance composition is 0.1-50 ng/ml, and the mass ratio is 0.00000001%-0.000005%. Preferably, the mass-volume concentration of the progesterone in the bioactive substance composition is 1-30 ng/ml, and the mass ratio is 0.0000001%-0.000003%. More preferably, the mass-volume concentration of the progesterone in the bioactive substance composition is 2-20 ng/ml, and the mass ratio is 0.0000002%-0.000002%. Preferably, the temperature for the mixing is 0-37° C.

The third object of the present invention is to provide a serum-free medium. The serum-free medium includes a basal medium and additional components, and the additional components include a bioactive substance composition in any form as described above or a bioactive substance composition prepared by the method described above. Preferably, the serum-free medium is a completely serum-free medium; preferably, the culture refers to the primary culture and secondary culture of cells or tissues; preferably, the culturing refers to maintaining or enhancing the proliferation and phenotype of cells or tissues. Preferably, the scores of each individual item in the cell common features and cell-specific phenotypes of the cells cultured in the serum-free medium reach their respective passing lines, and the total cell score reaches 60 points or more.

Further, the basic medium is selected from any one or more of DMEM low-sugar medium, DMEM high-sugar medium, DMEM/F12 medium, F12 medium, F10 medium, MEM medium, BEM medium, RPMI 1640 medium, Media 199 medium, IMDM medium, mTesR medium and E8 medium.

The fourth object of the present invention is to provide a method for preparing a serum-free medium. The preparation method includes the step of mixing a basic medium and additional components, and the additional components include any one form of bioactive substance composition as described above; preferably, the temperature of the mixing is 0-37° C.

The fifth object of the present invention is to provide a composition comprising at least one bioactive component and at least one additive. The bioactive component is selected from at least one of the bioactive substance composition as described above, a bioactive substance composition prepared by the method described above, and the serum-free medium prepared by the aforementioned method. Preferably, the cell common features and cell-specific phenotypes of the cells cultured in the composition reach their respective passing line and the total score of each individual item reaches 60 points or more. Preferably, the cell common features and cell-specific phenotypes of the cells cultured by the composition all reach their respective pass lines, and the total cell score reaches more than 80 points. Preferably, the cell common features and cell-specific phenotypes of the cells cultured by the composition all reach their respective pass lines, and the total cell score reaches more than 90 points. More preferably, the cell common features and cell-specific phenotypes of the cells cultured by the composition all reach their respective pass lines, and the total cell score reaches more than 100 points.

Further, in the composition, the additives are selected from any one or more of cell culture additives, growth factors, small molecule drugs, hormones, vitamins, wall promoting substances, macromolecular proteins, synthetic peptides, amino acids, lipids, enzymes, carbohydrate, pH regulating substances, trace elements and antibiotics.

Preferably, the cell culture additive includes any one or more of B27 cell culture additive, N2 cell culture additive, chemically defined lipid concentrate, ITS, and fatty acid additive. More preferably, calculated by the volume of the composition, the concentration of the cell culture additive in the composition is 0.1-5× (that is, 0.1-5 folds). More preferably, based on the volume of the composition, the concentration of the cell culture additive in the composition is 0.5-2× (that is, 0.5-2 folds).

Preferably, the growth factor is selected from any one or more of vascular endothelial growth factor, vascular endothelial growth factor synthetic peptide, epidermal growth factor, epidermal growth factor synthetic peptide, insulin-like growth factor, insulin-like growth factor synthetic peptide, nerve growth factor, nerve growth factor synthetic peptide, colony stimulating factor, colony stimulating factor synthetic peptide, growth hormone release inhibiting factor, growth hormone release inhibiting factor synthetic peptide. The mass volume concentration of the growth factor is 1-100 ng/ml. Preferably, the mass volume concentration of the growth factor is 1-50 ng/ml. More preferably, the mass volume concentration of the growth factor is 5-40 ng/ml.

Preferably, the small molecule drug is selected from GSK3 inhibitor. The GSK3 inhibitor is selected from CHIR99021. Preferably, the molar concentration of the small molecule drug is 0.1-10 μM. More preferably, the molar concentration of the small molecule drug is 0.1-5 μM.

Preferably, the amino acid is selected from any one or more of nonessential amino acids, L-glutamic acid and L-glutamine. More preferably, the molar concentration of the amino acid is 0.01-4 mM.

Preferably, the carbohydrate is sodium pyruvate; more preferably, the mass-volume concentration of the carbohydrate is 0.01-2 mM.

Preferably, the pH maintaining agent is selected from any one or more of 4-hydroxyethyl piperazine ethanesulfonic acid (HEPES) and L-glyceryl phosphate disodium salt water complexes. More preferably, the molar concentration of the pH maintaining agent is 1-20 mM.

Preferably, the adhesion promoting substance is selected from any one or more of laminin, fibronectin, vitronectin, collagen, gelatin and the synthetic peptide of the adhesion promoting substance. Preferably, the mass volume concentration range of laminin is 0.1-100 μg/ml. Preferably, the mass volume concentration range of fibronectin is 0.1-200 μg/ml. Preferably, the mass volume concentration range of vitronectin is 0.1-100 μg/ml. Preferably, the mass volume concentration range of collagen is 0.1-100 μg/ml. Preferably, the mass volume concentration range of gelatin is 0.1-100 μg/ml. Preferably, the mass volume concentration range of synthetic peptides of the adhesion promoting substance is 0.1-100 μg/ml.

Preferably, the antibiotic is selected from any one or more of penicillin, streptomycin and gentamicin. More preferably, the mass volume concentration range of the antibiotic is 50-100 μg/mL.

The sixth object of the present invention is to provide a preparation method of a composition, the preparation method comprising the step of mixing at least one bioactive component and at least one additive. The bioactive component is selected from at least one of the bioactive substance composition as described above, a bioactive substance composition prepared by the method described above, a serum-free medium as described above, the serum-free medium prepared by the method described above; preferably, the temperature of the mixing is 0-37° C.

The seventh object of the present invention is to provide a usage of a bioactive substance composition in any form as described above, a bioactive substance composition prepared by the method described above, a serum-free medium in any form as described above, a serum-free medium prepared by the method described above, and a composition in any form described above, and a composition prepared by the method described above.

Preferably, the cell is selected from any one or more of cells derived from tendons and/or ligaments, mesenchymal stem cells, meniscal stem cells, chondrocytes, skeletal stem cells, and muscle stem cells. Preferably, the tissue is the tissue derived from the musculoskeletal system. Preferably, the tissue derived from the musculoskeletal system is selected from tendon tissue, ligament tissue, meniscus tissue, cartilage tissue, fat tissue and muscle tissue. Preferably, the tissue and/or organ injury is the tissue and/or organ injury of the musculoskeletal system. Preferably, the tissue and/or organ injury of the musculoskeletal system is selected from at least one of tendon and/or ligament injury, cartilage injury, bone injury, muscle injury, skin injury, and blood vessel injury.

The eighth object of the present invention is to provide a cell culture method based on any one of the above-mentioned serum-free medium and/or composition. The culture method includes the step of contacting cells and/or tissues with a serum-free medium and/or composition. The serum-free medium is a serum-free medium in any form as described above, and a serum-free medium prepared by the method described above. The composition is any one form composition as described above, and a composition prepared by the method described above.

Preferably, the culture method is selected from suspension culture method and adherent culture method. Preferably, the adherent culture method is selected from the method of coating the surface of culture carriers by adhesion promoting substance, and the method of adding the adhesion promoting substance to the culture medium.

Preferably, the method of coating the surface of culture carriers by adhesion promoting substance comprises the following steps: 1) Treating the culture carrier with the adhesion promoting substance, preferably, the culture carrier is selected from at least one of the pore plate, culture dish, culture bottle, microcarrier, microsphere, microarray and bioactive material; 2) Inoculate cells and/or tissues into the culture carriers treated in step 1); 3) Add to the serum-free medium and/or the composition for culture.

More preferably, the method of adding the adhesion promoting substance to the culture medium comprises the following steps: 1) inoculating cells and/or tissues into a culture carrier, preferably, the culture carrier is selected from at least one of the pore plates, culture dishes, culture bottles, microcarriers, microspheres, microarrays, and bioactive materials; 2) Add the adhesion promoting substance directly to the serum-free medium and/or the composition, and then add it to the culture carrier in step 1) for cell culture.

Preferably, the cells are selected from any one or more of tendon and/or ligament derived cells, mesenchymal stem cells, meniscal stem cells, chondrocytes, skeletal stem cells, and muscle stem cells.

Preferably, the tissue is the tissue derived from the musculoskeletal system. Preferably, the tissue derived from the musculoskeletal system is selected from tendon tissue, ligament tissue, meniscus tissue, cartilage tissue, fat tissue and muscle tissue.

Preferably, the adhesion promoting substance is selected from any one or more of laminin, fibronectin, vitronectin, collagen, gelatin and the synthetic peptide of the adhesion promoting substance.

Preferably, the synthetic peptide of the adhesion promoting substance is a synthetic polypeptide, oligopeptide or amino acid sequence that can replace the adhesion promoting substance to promote cell adhesion, including any one or more of laminin synthetic peptide, fibronectin synthetic peptide, fibronectin synthetic peptide, RGD (Arg Gly Asp) peptide, KRSR (Lys Arg Ser Arg) peptide.

Preferably, the concentration range of laminin is 0.1-100 μg/ml, and/or the concentration range of fibronectin is 0.1-200 μg/ml, and/or fibronectin 0.1-100 μg/ml, and/or the concentration range of collagen is 0.1-100 mg/ml, and/or the concentration of gelatin is 0.1-100 mg/ml.

Preferably, the concentration range of laminin synthetic peptide is 0.1-100 μg/ml, and/or the concentration range of the fibronectin synthetic peptide is 0.1-200 μg/ml, and/or hyaluronan synthetic peptide 0.1-100 μg/ml, and/or the RGD (Arg-Gly-Asp) peptide concentration range is 50-1000 mg/ml, and/or the KRSR (Lys-Arg-Ser-Arg) peptide concentration range is 50-1000 mg/ml.

Preferably, the suspension culture method comprises the following steps: 1) Inoculate cells and/or tissues into low-adhesive or non-adhesive culture well plates, culture dishes, culture flasks, other culture carriers, cell dynamic culture bioreactors; 2) Add the serum-free medium and/or composition as described above to culture.

The ninth object of the present invention is to provide a cell and/or tissue that is obtained by culturing in the serum-free medium and/or the composition as described above. The serum-free medium is any one of serum-free medium as described above, the serum-free medium prepared by the method described above. The composition is any one form composition as described above, and a composition prepared by the method described above. Preferably, the cells are selected from any one or more of tendon and/or ligament derived cells, mesenchymal stem cells, meniscal stem cells, chondrocytes, bone stem cells, and muscle stem cells. Preferably, the tissue is the tissue derived from the musculoskeletal system. Preferably, the tissue derived from the musculoskeletal system is selected from tendon tissue, ligament tissue, meniscus tissue, cartilage tissue, fat tissue and muscle tissue. Preferably, the scores of each single item in the cell common features and cell-specific phenotypes of the cells reach their respective pass lines, and the total score of the cells reaches more than 60 points. Preferably, the scores of each single item in the cell common features and cell-specific phenotypes of the cells reach their respective pass lines, and the total score of the cells reaches more than 80 points. Preferably, the scores of each single item in the cell common features and cell-specific phenotypes of the cells reach their respective pass lines, and the total score of the cells reaches more than 90 points. More preferably, the scores of each single item in the cell common features and cell-specific phenotypes of the cells reach their respective pass lines, and the total score of the cells reaches more than 100 points.

Some technical terms involved in the present invention will be further explained below. These descriptions only use examples to illustrate how the method of the present invention is implemented, and does not constitute any limitation to the present invention.

Bioactive Substance Composition

The bioactive substance composition comprises fibroblast growth factor, platelet-derived growth factor, transforming growth factor-β, glucocorticoid, heparin or its salt, vitamin C or its derivative, transferrin, insulin, progesterone, putrescine or its salt, selenite.

The fibroblast growth factor (FGF), also known as heparin-binding growth factor, mainly includes two types of acidic and alkaline, and refers to a type of active protein or polypeptide substance that can promote the growth of cells. FGF includes but are not limited to FGF-basic, FGF1, FGF2, FGF4, FGF7, FGF12, FGF18, fibroblast growth factor synthetic peptides, etc.

The platelet-derived growth factor (PDGF) is a low-molecular-weight mitogen and a peptide regulator that stimulates the growth of connective tissue and other tissue cells. The platelet-derived growth factor family includes PDGF and vascular endothelial cytokine (VEGF). Each growth factor receptor is a tyrosine kinase (RTK) type receptor. Members of the platelet-derived growth factor family include: PDGFA, PDGFB, PDGFC, PDGFD, placental growth factor (PGF), vascular endothelial growth factor (VEGF), VEGF41, VEGFB, VEGFC, FIGF (VEGFD), homotype or heterodimers PDGF-AA, PDGF-BB, PDGF-AB, PDGF-CC and PDGF-DD formed by two polypeptide chains connected by disulfide bonds, and platelet-derived growth factor synthetic peptides.

The transforming growth factor-β (TGF-β) belongs to the multifunctional cytokine of the transforming growth factor superfamily, and has the functions of regulating cell growth and differentiation and maintaining cell phenotype, including but not limited to TGF-β1, TGF-β2, TGF-β3, transforming growth factor-β synthetic peptide, etc.

The glucocorticoid refers to a type of hormone that has important regulatory effects on cell growth, differentiation, metabolism, anti-inflammatory, and immunity. Common glucocorticoids include glucocorticoids, glucocorticoid-derived salts, glucocorticoid solvates, including but not limited to dexamethasone or its salt, dexamethasone solvate, hydrocortisone or its salt, hydrocortisone pine solvate, cortisone acetate, cortisone or its salt, hydroprednisolone sodium succinate, prednisone, betamethasone, betamethasone valerate, beclomethasone dipropionate, prednisolone acetate, prednisolone, etc.

The heparin or its salt refers to heparin or a salt of heparin. Heparin was first discovered from the liver and got its name. It is a mucopolysaccharide sulfate composed of glucosamine, L-iduronic acid, N-acetylglucosamine and D-glucuronic acid. The average molecular weight is 15 KDa, which is strongly acidic. Heparin is a natural anticoagulant substance in animals. It can promote cell proliferation during cell culture. Heparin or its salt includes but is not limited to heparin, heparin sodium, heparin calcium, and heparin.

The vitamin C or its derivatives refer to vitamin C, vitamin C salt and vitamin C solvate. Vitamin C (Vitamin C/ascorbic acid), also known as ascorbic acid, has an antioxidant effect, which can inhibit cell senescence, promote cell growth and phenotype maintenance. Vitamin C or its derivatives include, but are not limited to, vitamin C, ascorbyl glucoside, ethyl vitamin C, 3-o-ethyl ascorbic acid, vitamin C magnesium phosphate, vitamin C sodium phosphate, L-ascorbic acid 2-phosphate sesquimagnesium hydrate, vitamin C tetraisopalmitate, ascorbyl palmitate, ascorbic acid 2 phosphate 6 Palmitate, esterified vitamin C, other solvates of ascorbic acid, etc.

Additives

The additives include, but are not limited to, growth factors, small molecule drugs, hormones, vitamins, cell culture additives, adhesion-promoting substances, macromolecular proteins, synthetic peptides, amino acids, lipids, enzymes, carbohydrates, PH regulating substances, trace elements, antibiotics, etc.

The growth factor is a kind of polypeptide substance that regulates cell growth and other cell functions by binding to specific, high-affinity cell membrane receptors, and has an important regulatory effect on human immunity, hematopoietic regulation, tumorigenesis, inflammation and infection, wound healing, blood vessel formation, cell differentiation, cell apoptosis, morphogenesis, embryogenesis and other aspects. Growth factors are widely present in various tissues of the body, including mature tissues and embryonic tissues. They regulate the proliferation and differentiation of various cells through autocrine and/or paracrine methods, and many cells cultured in vitro can also release growth factors. In the present invention, microbial life activities are indispensable, and trace organic substances that the microbes cannot synthesize by themselves can be called growth factors. There are many kinds of growth factors, including fibroblast growth factor (FGF), platelet-derived growth factor (PDGF), and transforming growth factor (TGF), vascular endothelial growth factor (VEGF), insulin, epidermal growth factor (EGF), insulin-like growth factor (IGF), nerve growth factor (NGF), colony-stimulating factor (CSF) and growth hormone release inhibitor (somatostatin=SRIH) described in the above-mentioned bioactive substance composition. In this application, the fibroblast growth factor, platelet-derived growth factor, and transforming growth factor described in the above-mentioned bioactive substance composition are necessary for the activity of the bioactive substance composition or serum-free medium or composition. They work together with other components in the bioactive substance composition to realize cell/tissue culture and tissue repair. The functions of other growth factors described in the addition are mainly for further enhancing the activity of the composition, and are not necessary conditions.

The synthetic peptide refers to: 1) an amino acid sequence based on an effective fragment or a combination of fragments of a certain protein or polypeptide itself; or, 2) a polypeptide having the same amino acid sequence as a growth factor or adhesion factor prepared by a chemical synthesis method or oligopeptides, or, 3) mimetic peptides of a certain protein or polypeptide. These mimetic peptides can be obtained from peptide libraries using the receptors of known proteins or polypeptides, and their amino acid sequence is corresponding to the amino acid of the cytokine or adhesion factor. The sequence is different, but it has the activity of cytokine or adhesion factor, and has the advantage of small relative molecular mass. Among them, for the effective fragments of proteins or peptides, the corresponding amino acid sequence can be found according to the existing literature reports, and then synthesized by designing primers or entrusting biological companies to synthesize. If the effective fragments of proteins or peptides that have not been reported in the literature, they can be obtained by protein enzymatic hydrolysis and peptide mapping methods to find the optimized peptides, and then synthesize them by designing primers or entrust biological companies to synthesize them. In the same way, a mimetic peptide of a certain protein or polypeptide can be synthesized by a biological company based on the known amino acid sequence of the mimetic peptide of the protein or polypeptide. Synthetic peptides include, but are not limited to, growth factor synthetic peptides, and adhesion-promoting substances synthetic peptides. Growth factor synthetic peptides include but are not limited to one or more of fibroblast growth factor synthetic peptides, platelet-derived growth factor synthetic peptides, transforming growth factor synthetic peptides, epidermal growth factor synthetic peptides, insulin-like growth factor synthetic peptides, vascular endothelial growth factor synthetic peptides, nerve growth factor synthetic peptides and colony stimulating factor synthetic peptides. Adhesion promoting substance synthetic peptides include but are not limited to any one or more of laminin synthetic peptides, fibronectin synthetic peptides, vitronectin synthetic peptides, RGD (Arg-Gly-Asp) peptides, KRSR (Lys-Arg-Ser-Arg) peptides, synthetic peptides of other extracellular matrix components, and the like.

The small molecule drugs refer to a class of chemical small molecule drugs that are convenient to penetrate membranes and can enter cells through diffusion or carrier proteins to act on the intracellular pathway, including but not limited to any one or more of heparin or its salt, putrescine, selenious acid or its salt, CHIR99021, SB431542, etc.

The hormones include, but are not limited to any one or more of glucocorticoids, progesterone, insulin, progesterone, cortisol, corticosterone, triiodothyronine, and thyroxine (T3), etc.

The vitamins include, but are not limited to any one or more of vitamin A, vitamin B, vitamin C or derivatives thereof, vitamin D, vitamin E, vitamin K, vitamin H, vitamin P, vitamin PP, vitamin M, vitamin T, vitamin U, water-soluble vitamins, water-soluble vitamins, biotin, choline chloride, calcium D-pantothenate, folic acid, inositol, nicotinamide, pyridoxine hydrochloride, riboflavin, thiamine hydrochloride, coenzyme Q10, vitamin B12, putrescine dihydrochloride, tocopherol acetate, tocopherol, L-carnitine hydrochloride, acetyl-L-carnitine and the like.

The cell culture additive refers to a type of nutrient mixture that can provide nutrition to cells, promote cell proliferation and maintain phenotype, including any one or more of B27 cell culture additives (including but not limited to Gibco's B-27™ Supplement (50×), original concentration 50 times, ie 50×), N2 cell culture additives (including but not limited to Gibco's N-2 additive (100×), the original concentration is 100 times, ie 100×), chemically defined lipid concentrates (including but not limited to chemically defined lipid concentrates produced by Gibco), ITS (ie Insulin-Transferrin-Selenium mixed solution, including but not limited to Gibco, ITS produced by Sigma) and fatty acid additives (including but not limited to fatty acid additives produced by Gibco).

The adhesion promoting substance refers to a class of substances that can promote cell adhesion growth, including but not limited to any one or more of laminin, fibronectin, vitronectin, collagen, gelatin, and other extracellular matrix components.

The macromolecular protein includes, but is not limited to albumin, albumin-related protein and other non-growth factors that promote cell growth. Albumin-related proteins include, but are not limited to retinol binding protein, α-2-glycoprotein, transthyretin, heme binding globulin a, keratin precursors, and the like. Albumin can replace serum in cell culture, play a role in physiological and mechanical protection and as a carrier, and can promote the growth of mammalian cells and improve survival. In this application, albumin is an optional additive component, and the serum-free medium of the present invention can maintain cell proliferation and phenotype without adding albumin.

The amino acids include but are not limited to any one or more of MEM non-essential amino acid solution (Non-Essential Amino Acids, NEAA), glutathione, L-glutamine, L-carnitine, adenine, guanine, uracil, thymine, Cytosine, L-histidine, L-isoleucine, L-leucine, L-lysine, L-methionine, L-phenylalanine, L-threonine, L-tryptophan, L-valine, etc. Among them, the MEM non-essential amino acid solution includes 7 non-essential amino acids of L-alanine, L-glutamic acid, L-asparagine, L-aspartic acid, L-proline, L-serine and glycine, which can effectively improve the ratio of cell culture media, reduce the side effects of the cell's own production of non-essential amino acids during cell culture, and promote cell proliferation and metabolism. It is one of the commonly used additives in cell culture.

The lipids include, but are not limited to, any one or more of linoleic acid, oleic acid, linolenic acid, and cholesterol.

The enzymes refer to a type of reducing agent that scavenges free radicals, has strong antioxidant activity, can protect cells from cytotoxicity caused by glucose and glucose oxidase, and are mainly protein components. They include but not limited to any one or more of superoxide dismutase or its analogs, catalase or its analogs.

The carbohydrates refer to a class of substances that provide the main source of energy for cell growth, some of which are components that synthesize proteins and nucleic acids, including but not limited to any one or more galactose, glucose, ribose, deoxyribose, chondroitin sulfate, sodium pyruvate, and acetic acid.

The pH adjusting substance refers to a substance or mixture that maintains the pH stability of the cell culture environment and maintains the balance of cell osmotic pressure, including but not limited to any one or more of ethanolamine and its salts, 4-hydroxyethylpiperazine ethanesulfonic acid (HEPES) buffer, L-phosphate disodium glycerol hydrate, phenol red, sodium dihydrogen phosphate, trisodium phosphate, sodium pyrophosphate, potassium pyrophosphate, sodium bicarbonate, potassium hydroxide, ammonium hydroxide, triethanolamine and citric acid.

The trace elements are mainly involved in cell composition and metabolism, including but not limited to any one or more of iron, copper, zinc, cobalt, manganese, complex, selenium, iodine, nickel, fluorine, molybdenum, silver, tin, aluminum, barium, boron, rubidium, etc.

Musculoskeletal System

The generalized musculoskeletal system is composed of the central nervous system, peripheral nerves, nerve-muscle junctions; skeletal muscles; cardiopulmonary and metabolic support systems. The narrow movement system is composed of three organs: bone, bone connection (including tendon and ligament) and skeletal muscle. Bones are connected together in different forms to form skeletons. It forms the basic shape of the human body and provides attachment for the muscles. Under the innervation of the nerves, the muscles contract and pull the bones to which they are attached. The movable bone connection is used as the hub to produce lever motion.

Mesenchymal Stem Cells

Mesenchymal stem cells, also known as multipotent stromal cells, or MSCs for short, are a type of multipotent stem cells belonging to the mesoderm. They are mainly found in connective tissue and interstitium of organs. Their sources include: bone marrow, umbilical cord, fat, mucosa, bones, muscle, pulp, lung, liver, pancreas and other tissues, as well as amniotic fluid, amniotic membrane, placenta, etc. It is a type of cell that has the ability to self-renew and can differentiate into a variety of tissues such as fat, bone, cartilage, etc. under suitable conditions. Mesenchymal stem cells include, but are not limited to, bone marrow mesenchymal stem cells, umbilical cord mesenchymal stem cells, adipose stem cells, mucosal mesenchymal stem cells, dental pulp mesenchymal stem cells, amniotic fluid mesenchymal stem cells, amniotic membrane mesenchymal stem cells, placental mesenchymal stem cells, etc.

Tendon and/or Ligament-Derived Cells

Tendon and/or ligament-derived cells are a mixture of cells isolated from tendon or ligament tissue, and are highly expressing multiple tendon/ligament tissue-specific genes and proteins scleraxis (SCX), nestin (NES), tenomodulin (TNMD), thrombospondin-4 (THBS4), collagen type I alpha 1 (COL1, COL1A1), tenascin-C (TNC), which include tendon stem/progenitor cells (TSPCs, tendon derived stem cells, TDSCs, tendon stem cells, TSCs), tenocytes, tenoblasts, fibroblasts, ligament stem/progenitor cells, ligament cells, etc. They are the most ideal seed cell for tendon injury treatment.

Cell Common Features

The cell common features refer to the survival, proliferation and safety of cells. For stem cells, the cell common features in this application refer to the survival, proliferation, stem cell phenotype and safety of the stem cells, which are also called the stem cell common features or common features of stem cell.

Cell-Specific Phenotype

The cell-specific phenotype in this application refers to a unique phenotype that is distinguished from the features shared by stem cells. Take tendon stem cells as an example, the cell-specific phenotype is tendon phenotype and tendon differentiation ability, tendon and/or ligament repair ability in vivo.

Cultivation

Cultivation refers to a method that simulates the in vivo environment (sterile, suitable temperature, pH, and certain nutritional conditions, etc.) in vitro to make it survive, grow, proliferate and maintain its main structure and function (ie phenotype). The culture in the present invention refers to primary culture and secondary culture that maintain or enhance the proliferation and phenotype of cells or tissues.

Bioactive Materials

Bioactive materials refer to a class of natural or synthetic materials that are non-toxic or low-toxic to cells and/or tissues, have good biocompatibility, and can support cell and/or tissue culture.

Mass-Volume Concentration

The mass-volume concentration refers to the concentration expressed by the mass of the solute contained in the unit volume (1 m³, 1 L, 1 ml, etc.) solution, with the symbols g/m³, mg/L, mg/ml, g/ml. Mass-volume concentration=mass of solute/volume of solution. For example, if the mass of FGF2 in 1 ml serum-free medium is 10 ng, the concentration of FGF2 is 10 ng/ml.

Molar Concentration

Molar concentration generally refers to the amount concentration of a substance. The amount of substance concentration is defined as the amount of solute B in the solution divided by the volume of the mixture, represented by the symbol c(B). That is: in the formula, c(B)=n(B)/V, where Cb represents the quantity and concentration of the solute, n(B) represents the quantity of the solute B, and V represents the volume of the solution. If there are no special instructions, then it is considered that the solvent is water. The SI unit of the amount of substance is mol·m⁻³, and the common unit is mol·L⁻¹, abbreviated as M. mM, M and nM are commonly used in the patent of the present invention. The conversion formula of mass and molar concentration is: mass (mg)=molar concentration (mM)×volume (mL)×molecular weight (g/mol).

Conversion of Mass-Volume Concentration to Molar Concentration

The present invention relates to the conversion of molar concentration to mass-volume concentration. The specific conversion formula is mass-volume concentration (mg/ml)=molar concentration (mM)×molecular weight (g/mol). For example, the molecular weight of dexamethasone is 392.46, and the mass-volume concentration of 10 nM dexamethasone is 3.9246 ng/ml.

Various Concentration Calculation Methods Involved in the Present Invention

The mass-volume concentration ratio range of each component involved in the present invention is calculated by first converting the concentration of each component into a mass-volume concentration uniformly. The mass ratio involved in the present invention refers to the mass percentage, which is calculated by treating the solution as water. For example, the mass-volume concentration of fibroblast growth factor is 100 ng/ml, then the weight ratio in 1 ml solution (1 ml water is 1 g) is calculated as: (100 ng/1 g)*100%, that is (100 ng/109 ng)*100%=0.00001%. For example, if the molar concentration of dexamethasone is 10 nM, first convert it to a mass-volume concentration of 3.9246 ng/ml, then the weight ratio in 1 ml solution is calculated as: (3.9246 ng/1 g)*100%, that is (3.9246 ng/109 ng)*100%=0.00000039246%.

### The Beneficial Effects of the Present Invention

1) The present invention provides for the first time a highly active bioactive substance composition. The bioactive substance composition has a unique and indispensable composition of each component with a unique ratio, which makes its activity strong. The completely serum-free medium or composition prepared therefrom has clear ingredients and can realize completely serum-free primary culture and secondary culture of cells, especially overcoming the technical prejudice that serum must be involved in the primary culture of cells derived from tendons and/or ligaments in the prior art. The cultured cells can simultaneously meet the requirements of the number and quality of cells required for clinical treatment of tendon and/or ligament injuries, and have achieved unexpected technical effects. At the same time, the bioactive substance composition can also be used for the preparation of medicines for the treatment of tissue and/or organ injury in the body.

2) This invention provides for the first time a completely serum-free medium or composition with defined ingredients that promotes cell proliferation and phenotype maintenance. The serum-free medium or composition does not contain blood-derived substances such as serum, platelet-rich plasma and blood, and supports the primary culture and secondary culture of cells. Each additive component can effectively replace the serum component in the cell culture process through various mechanisms, so that the cell grows well, and the cell morphology, density, vitality, and function are significantly better than the serum-containing medium. The cells cultured in the serum-free medium or composition can simultaneously meet the quality and quantity requirements of the cells used in the clinical treatment of tissue injury. The total score of the cultured cells is 60 points and above, and can reach 100 points under the best conditions.

3) The serum-free medium or composition provided by the present invention can be used for in vitro culture containing cells derived from tendons and/or ligaments, mesenchymal stem cells, meniscal stem cells, chondrocytes, skeletal stem cells, and muscle stem cells.

4) The serum-free medium or composition of the present invention is particularly suitable for tendon and/or ligament-derived cell culture, especially for the first time to provide a completely serum-free medium with defined ingredients for tendon and/or ligament-derived cells. Taking tendon and/or ligament-derived cells as an example. Specifically: For the first time, complete serum-free culture of tendon and/or ligament-derived cells in vitro, including serum-free primary culture and secondary culture, achieves the following effects: a) Fast proliferation speed and short cell doubling time. Cells can be expanded by 5 times or more in one week. In a better state, the secondary culture can achieve 100,000-fold expansion in four weeks, and sufficient cell numbers can be obtained in a short period of time (FIG. 10-11, Table 1); b). High safety, normal cell karyotype, no serum residue, no Mycoplasma virus contamination (FIG. 13-14); c) Stem cell phenotypes such as maintained stem cell surface markers, clonogenic ability and triline differentiation ability (FIG. 15-17, Table 2, Table 3); d) Improved cell-specific phenotypes, such as the tendon line phenotype and tendon line differentiation ability of tendon and/or ligament-derived cells (FIG. 18-20, Table 4); e) Enhanced cell tissue repair ability (FIG. 21-25). Each individual score of the cultured cells reached the passing line and the total score reached 60 points or more (Table 5), which meets the quality and quantity requirements of clinical cell therapy.

### BRIEF DESCRIPTION OF THE DRAWINGS

FIG. 1 is the GPF fluorescence image of Scx-GFP tendon stem cells cultured by the existing culture technology and the statistical results of the Scx+ positive rate, showing that the prior art leads to the loss of the Scx phenotype, which is a specific marker of tendon lineage of tendon-derived cells (Biomaterials 2018; 172: 66-82).

FIG. 2 shows the detection results of DCN content in tendon-derived cells cultured with the existing culture technology, indicating that the existing culture technology has led to the loss of the DCN phenotype, which is a specific marker of tendon-derived cells, tendon lineage (Tissue Eng 2006; 12(7): 1843-9).

FIG. 3 is a transmission electron microscope image of the collagen transverse interface between the tendon and normal tendon tissue formed by the repair of tendon-derived cells cultured by the existing culture technology. The results show that the tissue repaired by cultured cells in the prior art is composed of a large amount of small collagen, which is much smaller than the diameter of normal tendon collagen.

FIG. 4 is a CT image of a tendon repaired by tendon-derived cells cultured in the prior culture technology, indicating that the tendon regenerated by cells cultured in the prior art has ectopic calcification and repair failure (STEM CELLS 2016; 34:1083-1096).

FIG. 5 is a broken line diagram of the number of cell proliferation in the prior art using insulin-like growth factor 1 and transforming growth factor R 3 to replace FBS for tenocyte culture, where the number of cell proliferation in the growth factor group is only ⅓ of that of the serum control group (Cells Tissues Organs 2013; 197:27-36).

FIG. 6 is a bar graph showing the collagen content of tendon cell culture using insulin-like growth factor 1 and transforming growth factor R 3 in place of FBS in the prior art. The collagen formation of the best combination of growth factor group is only ½ of that of the serum control group (Cells Tissues Organs 2013; 197:27-36).

FIG. 7 is a growth morphology diagram of primary tendon stem cells (P0) cultured in a serum-free medium in Example 1 of the present invention under an inverted microscope (4 times).

FIG. 8 is a diagram showing the growth morphology of tendon stem cells P1-P6 in the serum-free culture experimental group of Example 1 and the serum control group of Comparative Example 1 under an inverted microscope (4 times).

FIG. 9 is a diagram showing the growth morphology of tendon stem cells P3 in the serum-free culture experimental group of Example 1 and the serum control group of Comparative Example 1 under an inverted microscope (20 times).

FIG. 10 is a graph showing the multiplication of the cell proliferation multiples of each generation of tendon stem cells P1-P6 in the serum-free experimental group of Example 1 and the serum control group of Comparative Example 1 of the present invention.

FIG. 11 is a bar graph of the doubling time of tendon stem cells in the serum-free experimental group of Example 1 and the serum control group of Comparative Example 1 of the present invention.

FIG. 12 is a statistical diagram of the diameter of tendon stem cells in the serum-free experimental group of Example 1 and the serum control group of Comparative Example 1 of the present invention.

FIG. 13 is a diagram showing the karyotype analysis of tendon stem cells in the serum-free experimental group of Example 1 and the serum control group of Comparative Example 1 of the present invention.

FIG. 14 is a diagram showing the results of Mycoplasma detection in the serum-free medium, serum and Mycoplasma positive control in Example 1 of the present invention.

FIG. 15 shows the results of the ALP staining experiment of tendon stem cells in the serum-free experimental group of Example 1 and the serum control group of Comparative Example 1 to detect the osteogenic ability of the present invention.

FIG. 16 shows the results of Alcian Blue staining of tendon stem cells in the serum-free experimental group of Example 1 and the serum control group of Comparative Example 1 to test the cartilage ability of the present invention.

FIG. 17 is the results of oil red O staining of tendon stem cells in the serum-free experimental group of Example 1 and the serum control group of Comparative Example 1 of the present invention to detect the fat-forming ability.

FIG. 18 is a bar graph showing the relative expression of the tendon stem cells SCX, Nestin, TNMD and tendon-related genes in the serum-free experimental group of Example 1 and the serum control group of Comparative Example 1 of the present invention.

FIG. 19 is a diagram showing the results of Sirius scarlet staining of the tendon stem cells in the serum-free experimental group of Example 1 and the serum control group of Comparative Example 1 of the present invention after 14 days of induction.

FIG. 20 is a diagram showing the results of the tendon stem cells in the serum-free experimental group of Example 1 and the serum control group of Comparative Example 1 of the present invention.

FIG. 21 is a diagram showing the relative quantitative results of the ectopic formation of tendon tissue Scx, Nestin, Col1a1, and TNMD tendon line genes in nude mice in the serum-free experimental group of Example 1 and the serum control group of Comparative Example 1 of the present invention.

FIG. 22 is a graph showing the results of HE staining and Masson staining of tendon ectopic formation of tendon stem cells in nude mice in the serum-free experimental group of Example 1 and the serum control group of Comparative Example 1 of the present invention.

FIG. 23 is an immunofluorescence graph of the expression of ectopic tendon tissue Scx and Col1 tendon-related proteins in nude mice of the tendon stem cells in the serum-free experimental group of Example 1 and the serum control group of Comparative Example 1 of the present invention.

FIG. 24 is a graph showing the results of HE staining and Masson staining of rat in situ patellar tendon repair samples of tendon stem cells in the serum-free experimental group of Example 1 and the serum control group of Comparative Example 1 of the present invention.

FIG. 25 is an immunofluorescence graph of the Nestin tendon-associated protein expression of the rat in situ patellar tendon repair samples of the tendon stem cells in the serum-free experimental group of Example 1 and the serum control group of Comparative Example 1 of the present invention.

FIG. 26 is a diagram of the growth morphology of human ligament stem cells in the serum-free culture experiment group of Example 2 and the serum control group of Comparative Example 1 under an inverted microscope (20 times) of the present invention.

FIG. 27 is a diagram of the growth morphology of tendon stem cells in the serum-free culture experimental group of Example 3 and the serum control group of Comparative Example 1 under an inverted microscope (20 times) of the present invention.

FIG. 28 is the growth morphology diagram of human adipose-derived stem cells in the serum-free culture experimental group of Example 4 and the serum control group of Comparative Example 1 under an inverted microscope (20 times).

FIG. 29 shows the growth morphology of tendon stem cells under an inverted microscope (20 times) in the serum-free culture experimental group of Example 5, the serum-free control group of the comparative example 1, the commercialized Biological Industries MSC serum-free medium (BI SFM) group of comparative example 2, the commercialized Gibco MSC serum-free culture (ST SFM) group of the comparative example 3.

FIG. 30 shows a bar graph of the relative expression of genes related to tendon stem cells in the serum-free medium (SFM) group of Example 5, the serum-free medium (SCM) group of Comparative Example 1 and the commercialized bio-industrial MSC serum-free medium (BI SFM) in Comparative Example 2.

FIG. 31 is a three-dimensional growth morphology diagram of tendon stem cells in the serum-free culture experimental group of Example 6 and the serum control group of Comparative Example 1 under an inverted fluorescence microscope (10 times) of the present invention.

FIG. 32 is a three-dimensional growth morphology diagram of tendon stem cells in the serum-free culture experimental group of Example 7 and the serum control group of Comparative Example 1 of the present invention under an inverted fluorescence microscope (10 times).

FIG. 33 is a growth morphology diagram of human mesenchymal stem cells cultured in the serum-free culture experimental group in Example 8 and in Comparative Example 4 (SFM-Ctrl4) under an inverted microscope (4 times) of the present invention.

FIG. 34 is a bar graph of the cell proliferation ratio in the serum-free culture experimental group of Example 8 and in Comparative Example 4 (SFM-Ctrl4) after culturing human mesenchymal stem cells for 3 days.

FIG. 35 is a bar graph of the doubling time of human mesenchymal stem cells cultured in the serum-free culture experimental group in Example 8 and in Comparative Example 4 (SFM-Ctrl4) of the present invention.

FIG. 36 is a three-dimensional growth morphology diagram of human chondrocytes in the serum-free culture experimental group of Example 9 under an inverted fluorescence microscope (4 times).

FIG. 37 is a diagram of the cell growth morphology of human bone stem cells in the serum-free culture experimental group of Example 10 and the serum control group of Comparative Example 1 under an inverted fluorescence microscope (20 times) of the present invention.

FIG. 38 is a growth morphology diagram of tendon stem cells in the control medium containing only B27 cell culture additives in Comparative Example 5 under an inverted microscope (20 times).

FIG. 39 is the growth morphology of tendon stem cells in the serum-free medium of the comparative example 6 under the inverted microscope (4 times).

FIG. 40 is the growth morphology of tendon stem cells in the serum-free medium of the comparative example 7 under the inverted microscope (4 times).

FIG. 41 is the growth morphology of tendon stem cells in the serum-free medium of the comparative example 8 under the inverted microscope (4 times).

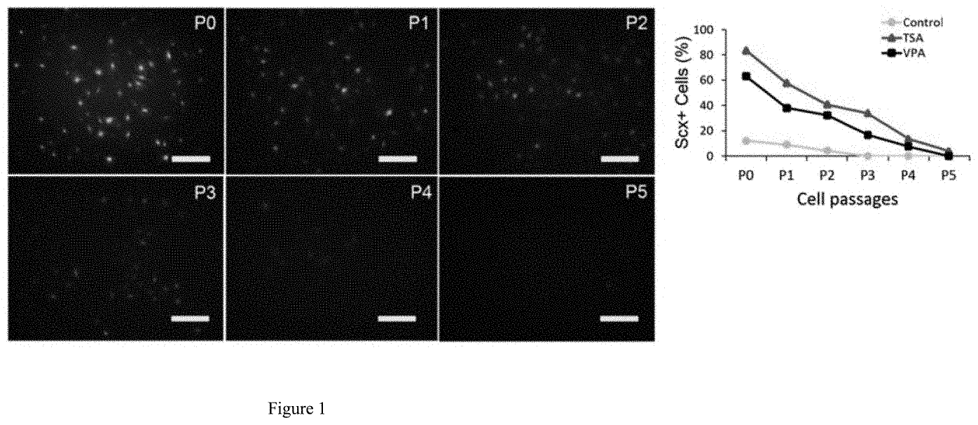

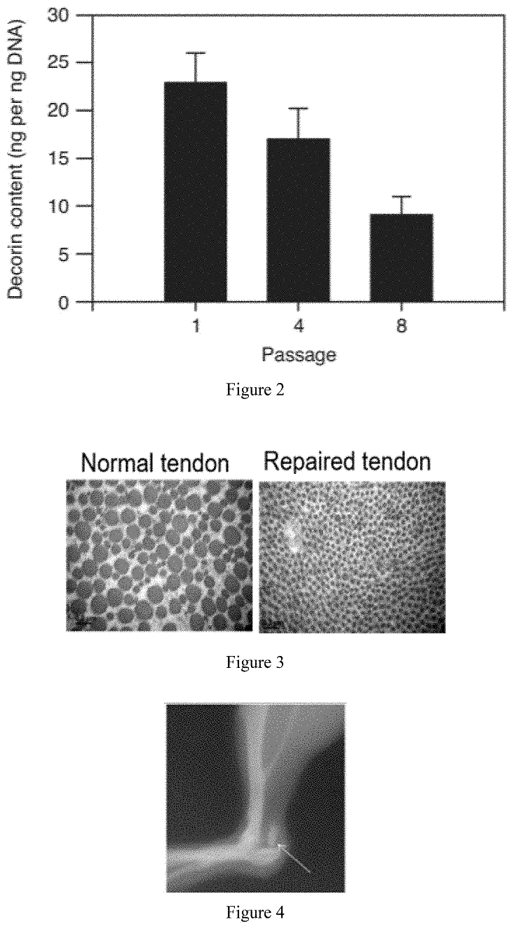

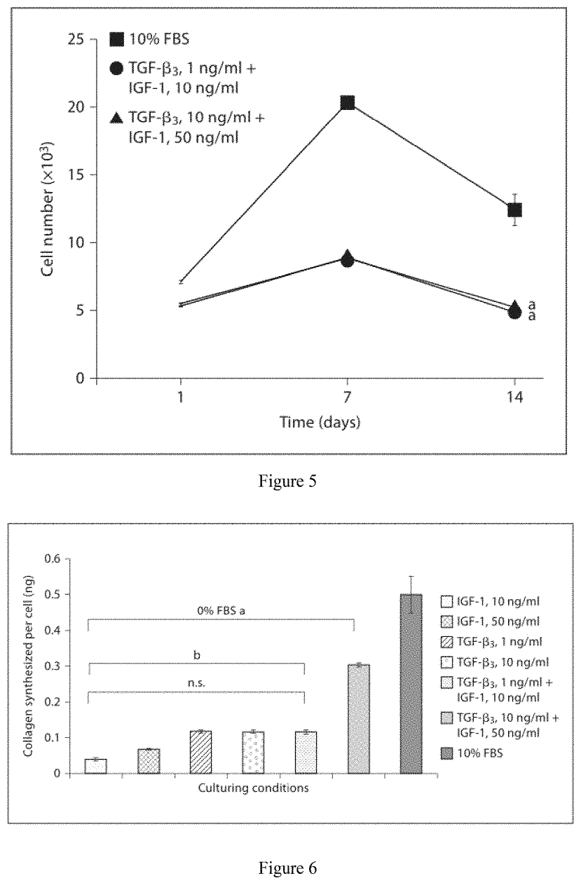

Table 1 shows the cell viability in each example and comparative example.

Table 2 shows the expression of cell surface markers in each example and comparative example.

Table 3 shows the statistics of cell clone formation ability in each example and comparative example.

Table 4 shows the percentage of Nestin+ cells cultured in each Example and Comparative Example.

Table 5 shows the scores of cells cultured in each example and comparative example.

### DETAILED DESCRIPTION OF THE DISCLOSURE

The present invention will be described in detail below in conjunction with specific embodiments. The following examples will help those skilled in the art to further understand the present invention, but do not limit this discovery in any form. It should be pointed out that for those of ordinary skill in the art, several modifications and improvements can be made without departing from the concept of the present invention.

These all belong to the protection scope of the present invention.

### Example 1

In this example, two groups of culture media were prepared, namely: serum-free medium and serum control culture medium, and the cultured cells were tendon stem cells (hTSPCs) extracted from human normal tendon tissue. The following are detailed experiments and tests step:

Medium Configuration

Serum-Free Medium (SFM)

The serum-free medium includes a basal medium and additional components. The basal medium is selected from DMEM/F12 medium, and every 500 mL of DMEM/F12 medium is supplemented with 5 mmol of HEPES, 10,000 U of penicillin, and 10,000 U of Streptomyces. The additional components are added so that the ratio of the concentration range of each component in the serum-free medium is: fibroblast growth factor:platelet-derived growth factor:transforming growth factor-β:glucocorticoid:heparin or its salt:vitamin C or its derivative:transferrin:insulin:progesterone:putrescine or its salt:selenite:epidermal growth factor:CHIR99021=10:10:5:2:1000:25000:2000:500:2:500:2:10:251. At the same time, the medium also contains 0.1 mM non-essential amino acids, 2 mM L-glutamic acid, 1 mM sodium pyruvate, and 1×B27 cell culture additive.

Further, the concentration of each component in the serum-free medium is as follows:

| | | |
|---|---|---|
| FGF2 | 20 | ng/ml |
| PDGF-BB | 20 | ng/ml |
| TGF-β 3 | 10 | ng/ml |
| Dexamethasone | 10 | nM |
| Heparin Sodium | 2 | μg/ml |
| Vitamin C | 50 | μg/ml |
| Transferrin | 4 | μg/ml |
| Insulin | 1 | μg/ml |
| Progesterone | 4 | ng/ml |
| Putrescine | 1 | μg/ml |
| Sodium selenite | 4 | ng/ml |
| Epidermal growth factor | 20 | ng/ml |
| CHIR99021 | 1 | μM |
| Non-essential amino acid | 1 | μM |
| L-glutamic acid | 2 | mM |
| Sodium pyruvate | 1 | mM |

| | |
|---|---|
| B27 Cell Culture Additive | 1X |

### Comparative Example 1

Serum Control Medium (SCM)

The serum control medium is selected from DMEM low-sugar medium, and every 500 mL of DMEM low-sugar medium is supplemented with 55 mL of fetal bovine serum, 5 mmol of HEPES, 10,000 U of penicillin, and 10,000 U of streptomycin.

### Experimental Example 1 of Biological Activity

Cultured Cell Processing

Primary Cell Isolation and Culture

Take the tendons and put them in a petri dish with 10% PS in PBS, 5% PS, 2% PS, and 1% PS in PBS for 1 min each for sterilization. Add the tendon tissue to the 0.2% collagenase solution, cut it into a paste, and make up the digestive juice to 10 ml. Immerse the tendon in the digestion medium, as evenly as possible, and place the medium in a 37° C. incubator. Blow away the tendons every hour until they are basically digested. Centrifuge the digested cell suspension at 1200 rpm for 5 min, discard the supernatant, and add it to a 10 cm culture dish coated with fibronectin, add 10 ml of SFM and place it in a 37° C., 5% CO2 cell incubator for culture. Change the solution once every 3-5 days. After the cells have adhered to 80-90%, perform cell digestion and passage for follow-up experiments, and freeze the excess cells.

Secondary Cell Culture

P1-P6 generation tendon stem cells were seeded at a density of about 9×10\hat{ }3/cm2 in well plates, dishes or flasks coated with 80 μg/ml fibronectin. Serum-free medium and serum were used, respectively. Culture the cells in the control medium and place them in a 37° C., 5% CO2 cell incubator. Change the medium every 3 days until it is almost full, and perform cell photographs, cell counts, gene expression detection, and three-line differentiation, immunofluorescence and other experiments.

Analysis of Results

Cell Morphology Observation

In the process of the above-mentioned cultured cells, the growth and morphological changes of tendon stem cells in the serum-free culture experimental group and the serum-culture control group were observed with an inverted phase contrast microscope, and photographed and recorded through a microscope.

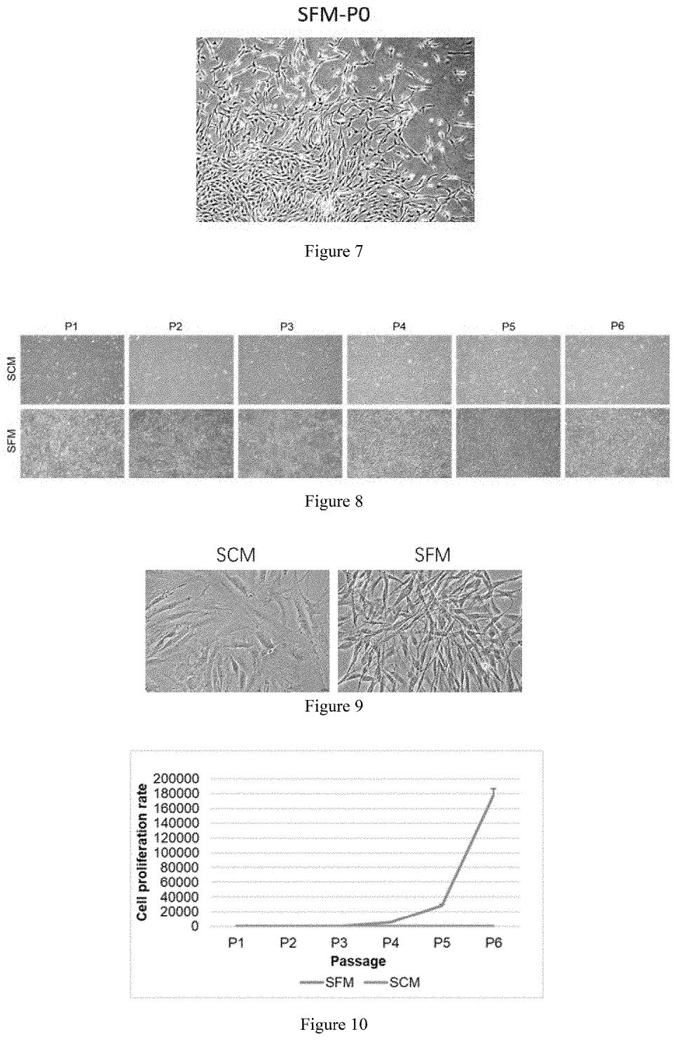

As shown in FIG. 7, under a 4× microscope, the primary cultured tendon stem cells cultured in serum-free culture grew into a single colony. The cells grew vigorously, were uniform in size, had a bright and abundant cytoplasm, and adhered well, indicating that the serum-free medium supported the primary culture of cells.

As shown in FIG. 8, 4× microscope showed the growth of P1-P6 generation cells when they were basically overgrown. The results showed that the tendon stem cells cultured in SFM (serum-free medium) grew vigorously, and the cell proliferation was significantly better than the SCM (serum medium) group.

As shown in FIG. 9, under a 20× microscope, compared with the SCM (serum medium) group, tendon stem cells cultured in SFM (serum-free medium) have more uniform cell morphology and size, rich cytoplasm, good adherence, and a larger number of cells.

Cell Count and Cell Proliferation Multiple Analysis

P1-P6 generation tendon stem cells were inoculated into a 10 cm culture dish coated with fibronectin at a density of about 9×10\hat{ }3/cm2, and the cells were cultured in serum-free medium and serum control medium until they were almost full. At this time, discard the culture medium, wash once with 1λPBS, trypsinize, centrifuge at 1200 rpm for 5 minutes, discard the supernatant, resuspend the pellet in 1 ml culture medium, and mix. The cell count uses trypan blue counting method. The cell suspension and 0.4% trypan blue solution were mixed 9:1 (final concentration of trypan blue 0.04%).

Cell count automatic technique:aspirate 20 μl of cell suspension and use a cell count to automatically count.

Manual counting method of hemocytometer: Pipette the cell suspension into the hemocytometer, observe and count under a microscope, the counting method is: (total number of cells in four large cells/4)×10⁴×dilution factor=cell number of cell suspension/mL.

If the cell membrane is intact and the cell is not stained with trypan blue, it is a normal cell; if the cell membrane is incomplete or ruptured, the trypan blue dye enters the cell and the cell turns blue, which is a necrotic cell.

Statistic cell viability: living cell rate (%)=total number of live cells/(total number of live cells+total number of dead cells)×100%.

Record the number of days of cell culture, the number of harvested cells, and the diameter of the cells at harvest, and draw related maps.

As shown in FIG. 10, the cell proliferation of the SFM (serum-free medium) group was 1.8×10\hat{ }5 times, while the SCM (serum medium) group only expanded by 40 times.

As a result, the cell proliferation rate of the SFM (serum-free medium) group was 4500 times that of the SCM group, and the proliferation rate of tendon stem cells in the SFM (serum-free medium) group was significantly higher than that of the SCM (serum medium) group.

As shown in Table 1, the viability of tendon stem cells in the SFM (serum-free medium) group was significantly higher than that of the SCM (serum medium) group.

Cell Doubling Time Analysis

P1-P6 generation tendon stem cells were inoculated into a 10 cm culture dish coated with fibronectin at a density of about 9×10\hat{ }3/cm2, and the cells were cultured in serum-free medium and serum control medium until they were almost overgrown. At this time, record the number of cell culture days, and calculate the cell doubling time according to the formula DT=t*[lg2/(lgNt−lgNo)]) (t is the culture time, No is the number of cells recorded for the first time, and Nt is the number of cells after t time), draw the relevant map.

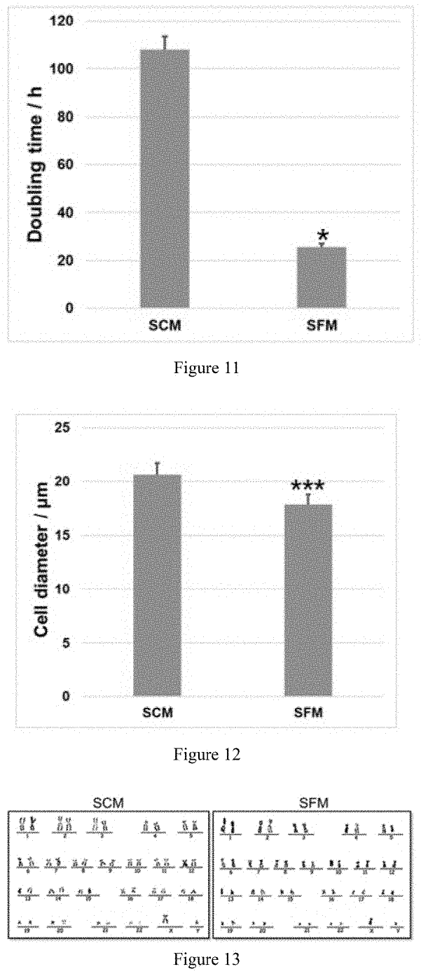

As shown in FIG. 11, the doubling time of tendon stem cells in the experimental group SFM (serum-free medium) was less than 30 hours, and was significantly lower than the SCM (serum medium) control group. The doubling time of the SCM group was 4.2 times that of the SFM group. The shorter the doubling time, the faster the cell proliferation, that is, the proliferation rate of SFM tendon stem cells in the experimental group was significantly faster than that of SCM control group. This shows that the serum-free medium can effectively replace the role of serum, and its ability to promote cell proliferation is significantly better than that of serum.

Cell Size Comparison

Plant tendon stem cells at a density of about 9×10\hat{ }3/cm2 in a well-coated 10 cm petri dish. Cultivate the cells under serum-free and serum-free conditions until they are almost full, discard the culture medium, wash once with 1λPBS, trypsinize, centrifuge at 1200 rpm, 5 minutes, discard the supernatant, resuspend the pellet in 1 ml culture medium, mix well, and aspirate Use a cell count to count 20 μl of cell suspension, record the cell diameter at harvest, and draw a bar graph.

As shown in FIG. 12, the cell diameter of the tendon stem cells harvested from the SFM experimental group was significantly smaller than that of the SCM control group. This shows that the SFM compared with the SCM control, the cells cultured in the serum-free medium are more in line with the features of stem cells.

Karyotype Analysis

Plant tendon stem cells at a density of about 9×10\hat{ }3/cm2 in a coated 10 cm petri dish, culture them under serum-free and serum-free conditions until they are almost fully grown, then send the cells to a genetic diagnosis company for karyotyping analyze.

As shown in FIG. 13, the results show that the tendon stem cells of the SFM experimental group and SCM control group are normal (the ratio of normal karyotype cells is greater than 90%). This indicates that the cells cultured in the serum-free medium are normal cells without karyotype mutation.

Mycoplasma Detection

Take the supernatant of the SFM group cell culture for 3 days and FBS, Mycoplasma positive control, and use the one-step rapid Mycoplasma detection kit for Mycoplasma detection. The main principle is that if the cell culture is contaminated by Mycoplasma, the conservative sequence of Mycoplasma DNA will be amplified in large quantities and quickly, so that the reaction solution will change from blue-purple to sky blue. The result can be distinguished by the naked eye without electrophoresis.

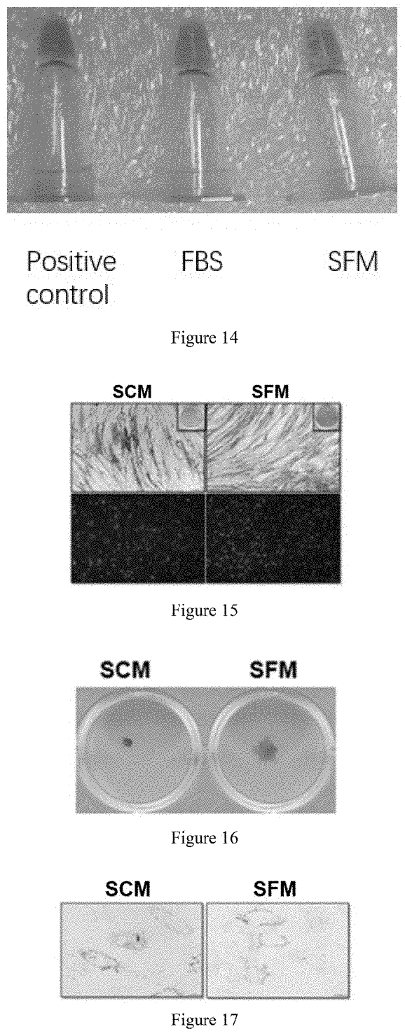

As shown in FIG. 14, the results showed that the SFM group detection reagent was original blue-purple, the Mycoplasma positive control was sky blue, and the FBS group detection color was slightly sky blue. This result indicates that the serum-free medium of the invention is free of Mycoplasma contamination and the cultured cells are safe, and the batch of FBS has slight Mycoplasma contamination, which also indicates that the addition of FBS to culture cells has a great risk of Mycoplasma contamination. The serum-free medium of the invention can avoid this risk.

Flow Cytometry to Detect the Expression of Stem Cell Surface Markers

Plant the P3 or P5 tendon stem cells at a density of about 9×10\hat{ }3/cm2 in a coated 10 cm petri dish, culture them under serum-free and serum-free conditions until they are almost full, discard the medium, and wash once with 1λPBS, trypsin digestion, centrifugation for 5 minutes, discard the supernatant, resuspend the pellet in the blocking solution, and block for 30 minutes, then separate the stem cells to stain the surface of the stem cells. After CD146, CD105, CD90, CD44, CD34, CD18 and other flow-type direct-labeled antibodies are stained for 30 minutes, add 1λPBS to mix and centrifuge to wash twice, add 500 ul PBS to resuspend on the machine to mix, and analyze the CD mark expression.

As shown in Table 2, CD105, CD90, and CD44 are positive expression markers of tendon stem cells, and CD34 and CD18 are negative expression markers of tendon stem cells. The results showed that the positive marker expression of the SFM experimental group was greater than 95%, and the negative marker expression was less than 1%, and it was more in line with the characteristics of tendon stem cells than the SCM control group. This shows that compared with SFM and SCM, the cells cultured in the serum-free medium are more in line with the features of stem cells.

Clone Forming Ability Test

P3 or P5 generation tendon stem cells were seeded in a 6 cm culture dish at a density of 100 cells/culture dish, in triplicate, cultured in serum-free and serum-free conditions for 10-12 days, stained with 1% crystal violet, several diameters >2 mm clone count.

As shown in Table 3, the results show that the clone formation ability of the SFM experimental group is significantly better than that of the SCM control group. This indicates that the serum-free medium is more consistent with the features of stem cells than the serum-free medium.

Three-Line Differentiation Ability Test

Take the P3-P5 generation serum-free medium and serum control medium to culture the cells and pass them to the well plates for osteoinduction, cartilage induction and fat induction respectively. The specific methods are as follows: The method and identification of directed differentiation into osteoblasts: collect and culture tendon stem cells, and then inoculate the digested cells in a 24-well plate at 1×10⁴ cells/cm2. After most of the cells adhere to the wall, the supernatant is removed and replaced. Use high-sugar DMEM culture medium containing 10% FBS, add the osteoinduction system and change the medium every 3 days for 14 days. Qualitatively using alkaline phosphatase (ALP) kit and alizarin red staining (ARS) to quantitatively detect the ability of cells to induce ossification. Use DAPI to label cell nuclei; then use 5% SDS-hydrochloric acid solution to elute Alizarin Red, read the microplate reader to obtain the OD value. The difference in the ratio of the OD value to the number of cells represents the difference in the quantitative osteogenic ability of different cells.

The method and identification of directional induction of chondrocyte differentiation: collect and culture TSPCs cells, digest the cells, drop the cells into the center of the 12-well plate at a concentration of 2×105 TSPCs/10 ul, and place them in the incubator until the cells adhere to the wall, add cartilage induction solution, change the whole solution every 2-3 days, fix it for Aclian blue staining after 2 weeks.

Methods and identification of directed differentiation into adipocytes: collect and culture TSPCs cells, and inoculate the digested cells in a 24-well plate at 1×104 cells/cm2. After most of the cells adhere to the wall, switch to high-glycemic DMEM medium containing 10% FBS and add the fat induction system. After maintaining for 2 weeks, observe the formation of intracellular fat droplets under a microscope, stained with oil red O (Oli redo). Use isopropanol to elute the red color, and read it under the microplate reader to obtain the value of its fat-forming ability (X±SD).

As shown in FIG. 15-17, the results show that the bone differentiation ability and chondrogenic differentiation ability of the SFM experiment is significantly better than the SCM control group. The lipid-forming ability of SFM and The SCM group is comparable. This indicates that the serum-free medium has a stronger ability to differentiate into three lines of cells cultured in the serum-free medium compared with the serum-containing control.

qPCR Detection of Gene Expression

Plant tendon stem cells in a well-coated 12-well plate, and culture them in serum-free medium and serum control medium until the 5th day, discard the medium, wash once with 1λPBS, and transfer the cells to the 12-well plate. Add 500 ul RNA cell lysate to the well, use RNA extraction kit to extract the cellular RNA, reverse transcribed into cDNA, and then load the sample on the machine for Qpcr to detect the relative expression of tendon-related genes in the cell. Analyze the results with the group as the abscissa and the relative gene expression as the ordinate, and draw a histogram of the relative expression of the gene.

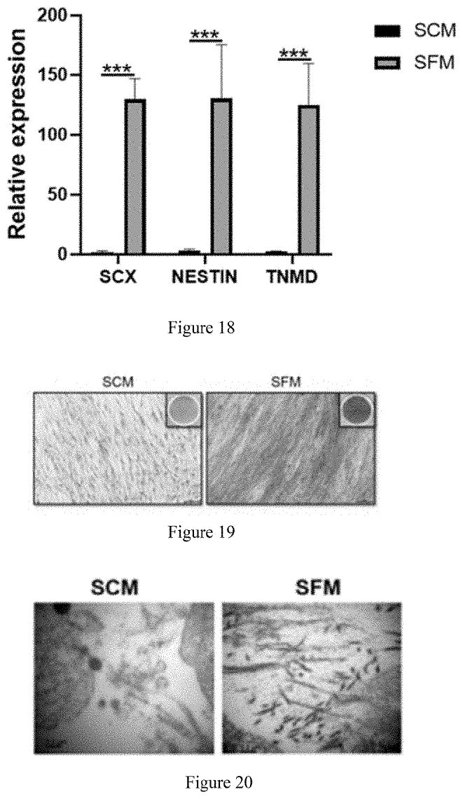

As shown in FIG. 18, comparing SFM and SCM control group, SCX, nestin, and TNMD tendon-related genes are significantly higher in the serum-free experimental group. Therefore, it indicates that the SFM is more conducive to the maintenance of tendon lineage phenotype of tendon stem cells than SCM.

In Vitro Tendon Differentiation Ability Test

Take the P3-P5 generation serum-free medium and serum control medium to culture the cells in a 12-well plate at 4×10\hat{ }4 cells/well, with three multiple wells in each group, respectively in serum-free medium and serum control medium. When cultured in serum-free medium and serum control medium until they are almost overgrown, replace the tendon induction medium, change the medium once every 2-3 days. One to two weeks after the induction, the cells were stained with Sirius scarlet, and the cell sheets were rolled into a transmission electron microscope to evaluate the formation of collagen.

As shown in FIG. 19 by Sirius Scarlet staining, compared with the SFM control group, the amount of collagen formation in the SFM experimental group was significantly increased. It shows that under the condition of tendon line induction, the tendon stem cells cultured in the serum-free medium have stronger tendon line differentiation ability than the tendon stem cells in the serum medium.

As shown in FIG. 20, the results of transmission electron microscopy showed that compared with the control group of SFM (serum-free medium), the amount of collagen formed in the experimental group of SFM (serum-free medium) was significantly increased, and the diameter of the collagen formed was significantly larger than that in the control group. It shows that under the condition of tendon line induction, the tendon stem cells cultured in the serum-free medium have stronger tendon line differentiation ability than the tendon stem cells in the serum medium.

Flow Analysis to Detect Nestin Expression

Plant the P3 or P5 tendon stem cells at a density of about 9×10\hat{ }3/cm2 in a coated 10 cm petri dish, culture them under serum-free and serum-free conditions until they are almost full, discard the medium, wash once with 1λPBS, digest with trypsin, centrifuge for 5 minutes, discard the supernatant, resuspend the pellet in the blocking solution, and block for 30 minutes, break the membrane, separate the tube for Nestin staining of stem cells, after 30 minutes, add 1λPBS to mix and centrifuge to wash twice, add 500 ul PBS to resuspend and mix on the machine. Finally, analyze the expression of Nestin.

As shown in Table 4, the results show that the Nestin positive marker expression in the SFM experimental group is greater than 30%, while the Nestin positive marker expression in the SCM control group of the comparative example 1 is less than 5%. This shows that the phenotype of the cell tendon line cultured in the SFM experimental group is significantly higher than that of the SCM control group.

In Vivo Tendon Formation Ability Test

Take the serum-free medium and the serum control medium to culture the P5 generation cells at a density of about 9×10\hat{ }3/cm2 and inoculate them in a coated 10 cm petri dish, and cultivate them in the serum-free medium and the serum control medium respectively. When it is basically overgrown, it is cultured with tendon induction medium, and the medium is changed once every 2-3 days. Two weeks after induction, they were rolled into a cell sheet and implanted under the skin of the back of nude mice. The samples were collected two weeks later. The tendon formation of the implanted cells was evaluated by HE staining, masson staining, immunofluorescence staining, qPCR and other experiments.

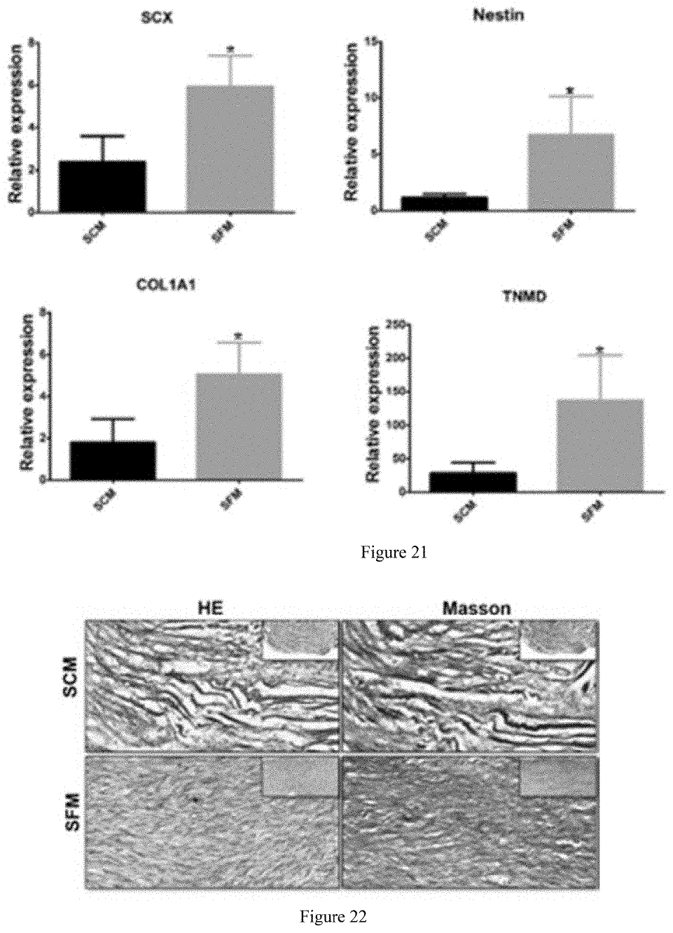

As shown in FIG. 21, the QPCR results showed that compared with SCM control group, the expression of tendon-related genes such as SCX, Nestin, TNMD, COL1A1, etc., was significantly increased in the serum-free experimental group. It shows that the tendon stem cells cultured in the serum-free medium have stronger ability to differentiate tendon lineage, and the tendon tissue formed in the body is more mature.

As shown in FIG. 22, the histological results showed that compared with the SFM control group, the collagen in the tendon tissue formed in the SFM experimental group is arranged more neatly and densely, indicating that the tendon stem cells cultured in the serum-free medium have a stronger ability to form tendons in vivo.

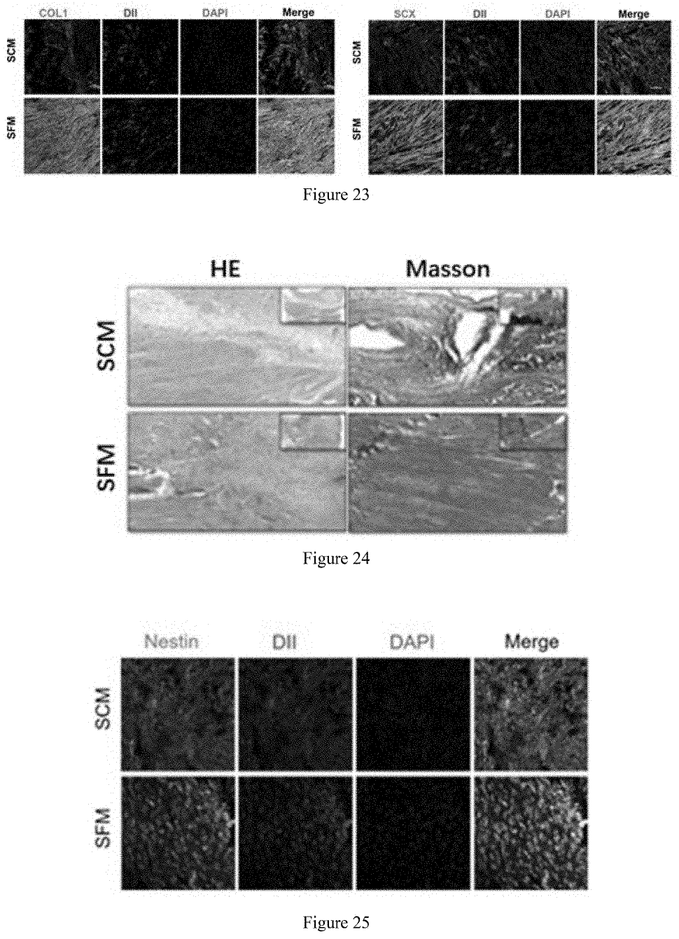

As shown in FIG. 23, the immunofluorescence results showed that compared with the SFM (serum-free medium) control group, the expression of tendon-related proteins such as SCX and COL1A1 were significantly increased in the serum-free experimental group. The serum-free medium is more conducive to the differentiation of tendon stem cells into tendon lineage to form tendon tissue than serum medium.

Evaluation of In Situ Tendon Repair Ability In Vivo

Take the serum-free medium and the serum control medium to culture the P5 generation cells at a density of about 9×10\hat{ }3/cm2 and inoculate them in a coated 10 cm petri dish, change the medium once every 2-3 days, respectively in the serum-free medium and the serum control medium. After being cultured in serum-free medium and serum control medium until they are almost fully grown, the cells are digested into single cells, mixed with fibrin gel to form a gel, and then implanted into the local defect of the rat patellar tendon. After four or eight weeks, samples will be collected, and the formation of tendon of implanted cells will be evaluated by HE staining, masson staining, immunofluorescence staining and other experiments.

As shown in FIG. 24, the histological results show that compared with the SCM group, tendon tissue collagen in the SFM experimental group is more orderly and denser, indicating that the tendon stem cells cultured in the serum-free medium have a stronger ability to repair tendon in situ in vivo.

As shown in FIG. 25, the immunofluorescence results showed that compared with the SCM group, the expression of the tendon-related protein Nestin was significantly increased in the serum-free experimental group. Therefore, it indicates that the serum-free medium is more conducive to the differentiation of tendon stem cells into tendon lineage to form tendon tissue than serum medium.

### Example 1 of Cell Score Calculation

According to the results of “Analysis of Cell Doubling Time” in Example 1, the doubling time of the cells cultured in the serum-free medium in Example 1 is less than 30 hours, so the cell proliferation rate is scored as 30 points. From the results of Example 1 “Detection of stem cell surface marker expression by flow cytometry”, it can be seen that the positive marker expression of cells cultured in serum-free medium is greater than 95%, and the negative marker expression is less than 1%, which is higher than that of SCM control. The cells in SFM group are more in line with the characteristics of tendon stem cells, indicating that the expression of surface markers on the cell stem cells cultured in the serum-free medium in Example 1 is increased. From the analysis result of “Clonogenic Ability Determination” in Example 1, it can be seen that the clonal forming ability of the SFM experimental group (25 cells/well) is significantly better than that of the SCM control group (12 cells/well). According to the analysis results of the “Triline Differentiation Ability Test” in Example 1, it can be seen that the bone differentiation ability and chondrogenic differentiation ability of the SFM experiment is significantly better than the SCM control group, and lipid-forming ability of SFM is equivalent to that of SCM group. This indicates that the serum-free medium has a stronger ability to differentiate into three lines of cells cultured in the serum-free medium compared with the serum-containing control. Based on the above results, it can be seen that using the conventional serum medium cultured cells in the prior art as a control, the surface marker expression, cloning ability and triline differentiation ability of the stem cells obtained by the serum-free medium culture of Example 1 are all improved. Therefore, the score for stem cell phenotype is 20 points.

It can be seen from the “karyotype analysis” result of Example 1 that the karyotype of the cells cultured in the serum-free medium of Example 1 is normal. It can be seen from the results of “Mycoplasma Detection” in Example 1 that the serum-free medium of Example 1 is free of Mycoplasma contamination, and the cultured cells are safe, and this batch of FBS has slight Mycoplasma contamination. In combination with the serum-free medium of the present invention, it is a completely serum-free medium, which can realize cell primary culture and secondary culture. Moreover, no serum participates in the whole process, so there is no serum residue. In summary, the karyotype of the cells obtained by the serum-free medium culture in Example 1 is normal, there is no serum residue, and no Mycoplasma contamination, so the safety score is 10 points.

From the results of “qPCR detection of gene expression” in Example 1, compared with the SCM control group, the SCX, Nestin, and TNMD tendon-related genes of cells cultured in serum-free medium were all significantly highly expressed in the serum-free experimental group. It can be seen from the results of Example 1 “Detection of Nestin Expression by Flow Cytometry” that the Nestin positive rate of cells cultured in the serum-free medium of Example 1 can reach 94%. And the “in vitro tendon differentiation ability test” results also show that the collagen-forming ability of the cells cultured in the serum-free medium of Example 1 is significantly higher than that of the serum control group. To sum up, the phenotype and differentiation ability of tendon line obtained by the serum-free medium of Example 1 were significantly improved compared with the serum control group. In addition, the three tendon line related genes of SCX, Nestin and TNMD were highly expressed, and the positive rate of Nestin was greater than 90%. Therefore, the score for tendon phenotype and tendon differentiation ability is 20 points.

From the results of Example 1 “In vivo tendon formation ability detection” and “In vivo in situ tendon repair ability evaluation”, the histological results show that compared with the serum control group (SCM group), the collagen of tendon tissue formed by repairing cells cultured in serum-free medium is arranged more neatly and densely, without bone, cartilage, muscle and other non-tendon tissues, which is closer to normal tissue morphology. Therefore, the ability to repair tendons and/or ligaments in the body is scored 20 points.

In summary, the total score of the cells obtained by the serum-free culture in Example 1 is 100 points, and at the same time, it meets the cell quantity and quality requirements for clinical cell therapy of tendon and or ligament injuries.

### Example 2

In this embodiment, two groups of culture media are prepared, namely: serum-free medium and serum control culture medium, and the cultured cells are ligament stem cells obtained by separation and culture of human ligament tissue. The following are detailed experiments and detection steps:

Medium Configuration

Serum-Free Medium (SFM)

The serum-free medium includes a basal medium and additional components; the basal medium is selected from F12 medium, and 5 mmol of HEPES, 10000 U of penicillin and 10000 U of streptomycin are added to every 500 mL of F12 medium. Moreover, additional components were added to the medium, and the ratio of the concentration range of each component in the serum-free medium is: fibroblast growth factor:platelet-derived growth factor:transforming growth factor-β:glucocorticoid:heparin or its salt:vitamin C or its derivatives:transferrin:insulin:progesterone:putrescine or its salt:selenite=15:10:6:2:1500:25000:75000:3000:4:7:4.

Further, the concentration of each component in the serum-free medium is as follows:

| | | |
|---|---|---|
| FGF-basic | 30 | ng/ml |
| PDGF-AA | 10 | ng/ml |
| PDGF-BB | 10 | ng/ml |
| TGF-β 1 | 3 | ng/ml |
| TGF-β 3 | 3 | ng/ml |
| Dexamethasone | 10 | nM |
| Heparin sodium | 3 | μg/ml |
| Vitamin C | 50 | μg/ml |
| Transferrin | 150 | μg/ml |
| Insulin | 6 | μg/ml |
| Progesterone | 8 | ng/ml |
| Putrescine | 14 | μg/ml |
| Sodium selenite | 8 | ng/ml |

### Example 2 Experimental Example of Biological Activity

Cultured Cell Processing

P3-P6 generation human ligament stem cells were seeded into wells coated with 100 μg/ml laminin, 200 μg/ml fibronectin and 100 ug/ml vitronectin at a density of about 9×10\hat{ }3/cm² in a plate or a petri dish. Culture the cells in a serum-free medium and a serum control medium respectively, and place them in a 37° C., 5% CO2 cell incubator. Change the medium every 3 days until it is almost full, and take a photo of the cells to observe the cell growth condition.

Analysis of Results

Cell Morphology Observation

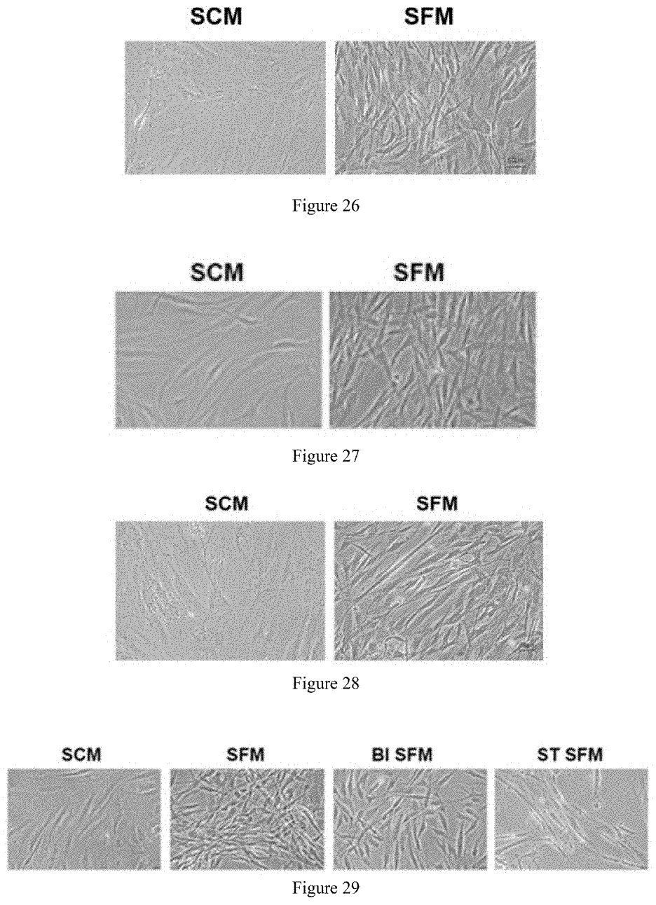

The method is the same as in Example 1. As shown in FIG. 26, the 20× microscope showed the cell morphology when the cells were basically overgrown. The results showed that compared with the SCM group, the human ligament stem cells cultured in SFM, human ligament stem cells cultured in SFM (serum-free medium) have more uniform morphology and size, rich cytoplasm, good adhesion, and a larger number.

Flow Cytometry to Detect the Expression of Stem Cell Surface Markers

The method is the same as in Example 1. As shown in Table 2, CD105, CD90, and CD44 are positive expression markers of human ligament stem cells, and CD34 and CD18 are negative expression of human ligament stem cells. The results showed that the positive marker expression of the SFM experimental group was greater than 95%, and the negative marker expression was less than 1%. Moreover, the cells in the SFM experimental group were more consistent than that of the SCM (serum-free medium) control group. This indicates that compared with SFM and SCM control, the cells cultured in the serum-free medium are more in line with the features of stem cells.

Clone Forming Ability Test

The method is the same as in Example 1. As shown in Table 3, the results showed that the clone formation ability of the SFM (serum-free medium) experimental group were significantly better than that of the SCM (serum medium) control group. This indicates that the serum-free medium is more in line with the features of stem cells than the serum-free medium.

### Example 3

In this example, two groups of culture media were prepared, namely: serum-free medium and serum control culture medium, and the cultured cells were tendon stem cells (hTSPCs) extracted from human normal tendon tissue. The following are detailed experiments and tests step:

Medium Configuration

Serum-Free Medium (SFM)

The serum-free medium includes a basal medium and additional components; the basal medium is selected from F12 medium, and 5 mmol of HEPES, 10000 U of penicillin and 10000 U of streptomycin are added to every 500 mL of F12 medium. Moreover, additional components were added to the medium, and the ratio of the concentration range of each component in the serum-free medium is: fibroblast growth factor:platelet-derived growth factor:transforming growth factor-β:glucocorticoid:heparin or its salt:vitamin C or its derivatives:transferrin:insulin:progesterone:putrescine or its salt:selenite=50:50:40:11:1000:100000:300,000:25000:25:25000:25. At the same time, the medium also contains 0.1 mM non-essential amino acids, 0.1 mM L-glutamic acid, 0.1 mM sodium pyruvate, and 0.1× B27 cell culture additive.

Further, the concentration of each component of the serum-free medium is as follows:

| | | |
|---|---|---|
| FGF2 | 20 | ng/ml |
| FGF4 | 20 | ng/ml |
| FGF18 | 10 | ng/ml |
| PDGF-AA | 25 | ng/ml |
| PDGF-AB | 25 | ng/ml |
| TGF-β 1 | 20 | ng/ml |
| TGF-β 2 | 20 | ng/ml |
| Dexamethasone | 15 | nM |
| Hydrocortisone | 14 | nM |
| Heparin | 1 | μg/ml |
| Vitamin C | 50 | μg/ml |
| Vitamin C sodium phosphate | 50 | μg/ml |
| Transferrin | 300 | μg/ml |
| Insulin | 25 | μg/ml |
| Progesterone | 25 | ng/ml |
| Putrescine | 15 | μg/ml |
| Putrescine dihydrochloride | 10 | μg/ml |
| Sodium selenite | 25 | ng/ml |
| Non-essential amino acid | 0.1 | mM |
| L-glutamic acid | 0.1 | mM |
| Sodium pyruvate | 0.1 | mM |

| | |
|---|---|
| B27 Cell Culture Additive | 0.1X |

### Example 3 Experimental Example of Biological Activity

Cultured Cell Processing

P3-P6 generation tendon stem cells were inoculated into 40 mg/ml gelatin-coated well plates, petri dishes or flasks at a density of about 9×10\hat{ }3/cm2. Culture the cells in a serum-free medium and a serum control medium respectively, and place them in a 37° C., 5% CO2 cell incubator, change the medium every 2-3 days, cultivate until the cells are basically full, and take pictures to observe cell growth.

Analysis of Results

Cell Morphology Observation

The method is the same as in Example 1. As shown in FIG. 27, under a 20× microscope, the cell morphology when the cells were basically overgrowth was shown. The results showed that compared with SCM group, the tendon stem cells cultured in SFM grew vigorously, and the cell proliferation was significantly better than that of the SCM group. The cell morphology and size were more uniform, the cytoplasm was transparent and abundant, and the adherence was good

Flow Cytometry to Detect the Expression of Stem Cell Surface Markers

The method is the same as in Example 1. As shown in Table 2, CD105, CD90, and CD44 are positive expression markers of tendon stem cells, and CD34 and CD18 are negative expression markers of tendon stem cells. The results showed that the positive marker expression of the SFM experimental group was greater than 95%, and the negative marker expression was less than 1%. Moreover, the cells in SFM experimental group were more in line with the characteristics of tendon stem cells than the cells in SCM control group. Therefore, compared with the SCM control, the cells cultured in the serum-free medium are more in line with the features of stem cells.

Clone Forming Ability Test

The method is the same as in Example 1. As shown in Table 3, the results show that the clone formation ability of the SFM experimental group is significantly better than that of the SCM control group. Therefore, compared with the SCM control, the cells cultured in the serum-free medium are more in line with the features of stem cells.

### Example 4

In this example, two groups of culture media were prepared, namely: a serum-free medium and a serum control medium, and the cultured cells were adipose-derived stem cells (ADSCs) extracted from human fat. The following are detailed experiments and detection steps:

Medium Configuration

Serum-Free Medium (SFM)

The serum-free medium includes a basal medium and additional components; the basal medium is selected from F12 medium, and 5 mmol of HEPES, 10000 U of penicillin and 10000 U of streptomycin are added to every 500 mL of F12 medium. Moreover, additional components were added to the medium, and the ratio of the concentration range of each component in the serum-free medium is: fibroblast growth factor:platelet-derived growth factor:transforming growth factor-β:glucocorticoid:heparin or its salt:vitamin C or its derivative:transferiron Protein:insulin:progesterone:putrescine or its salt:selenite=1:1:1:1:10:10:10:1:1:1:1. At the same time, the medium also contains 1 mM non-essential amino acids, 4 mM L-glutamic acid, 2 mM sodium pyruvate, 2× B27 cell culture additive, 1 μg/ml vitronectin, 1 μg/ml fibronectin, 1 μg/ml laminin.

| bFGF | 10 | ng/ml |
|---|---|---|
| PDGF-AA | 10 | ng/ml |
| TGF-β 2 | 10 | ng/ml |
| Dexamethasone | 25 | nM |
| Heparin sodium | 0.1 | μg/ml |
| Vitamin C | 0.1 | μg/ml |
| Transferrin | 0.1 | μg/ml |
| Insulin | 0.01 | μg/ml |
| Progesterone | 10 | ng/ml |
| Putrescine | 0.01 | μg/ml |
| Sodium selenite | 10 | ng/ml |
| Non-essential amino acid | 1 | mM |
| L-glutamic acid | 4 | mM |
| Sodium pyruvate | 2 | mM |
| B27 Cell Culture Additive | 2X | |
| Vitronectin | 1 | μg/ml |
| Fibronectin | 1 | μg/ml |
| Laminin | 1 | μg/ml |

### Experimental Example 4 of Biological Activity

Cultured Cell Processing

P3-P6 generation adipose-derived stem cells were seeded into a well plate, petri dish or flask at a density of about 5×10\hat{ }3/cm2, and the cells were cultured with serum-free medium and serum control medium, respectively. The cells were cultured in a 37° C., 5% CO2 cell incubator, and the medium was changed every 3 days until the cells were basically overgrown, and the cells were photographed to observe the growth of the cells.

Analysis of Results

Cell Morphology Observation

The method is the same as in Example 1. As shown in FIG. 28, a 20× microscope shows the cell morphology when the cells are basically overgrown. The adipose-derived stem cells cultured in SFM grew vigorously, and the cell proliferation was significantly better than that of the SCM group. In addition, the adipose-derived stem cells cultured in SFM have a more uniform cell morphology and size, a translucent and rich cytoplasm, and a good adherence to the wall.

Flow Cytometry to Detect the Expression of Stem Cell Surface Markers

The method is the same as in Example 1. As shown in Table 2, CD105, CD90, and CD44 are positive expression markers of tendon stem cells, and CD34 and CD18 are negative expression markers of tendon stem cells. The results showed that the positive marker expression of the SFM experimental group was greater than 95%, and the negative marker expression was less than 1%. Moreover, the cells in SFM experimental group were more in line with the characteristics of tendon stem cells than the cells in SCM control group. Therefore, compared with the SCM control, the cells cultured in the serum-free medium are more in line with the features of stem cells.

Clone Forming Ability Test

The method is the same as in Example 1. As shown in Table 3, the results show that the clone formation ability of the SFM experimental group is significantly better than that of the SCM control group. Therefore, compared with the SCM control, the cells cultured in the serum-free medium are more in line with the features of stem cells.

### Example 5

In this example, four groups of culture media were prepared, namely: serum-free medium, serum control medium, commercial Biological Industries MSC serum-free medium, commercial Gibco MSC serum-free culture, and the cultured cells were human Tendon stem cells (hTSPCs) extracted from normal tendon tissues, the following are the detailed experiments and detection procedures:

Medium Configuration

Serum-Free Medium (SFM)

The serum-free medium includes a basal medium and additional components; the basal medium is selected from F12 medium, and 5 mmol of HEPES, 10000 U of penicillin and 10000 U of streptomycin are added to every 500 mL of F12 medium. Moreover, additional components were added to the medium, and the ratio of the concentration range of each component in the serum-free medium is: fibroblast growth factor:platelet-derived growth factor:transforming growth factor-β:glucocorticoid:heparin or its salt:vitamin C or its derivative:transferrin:insulin:progesterone:putrescine or its salt:selenite salt:epidermal growth factor:CHIR99021=30:30:5:2:2000:80000:50000:100:5:100:5:10:1004. At the same time, the medium also contains 0.1 mM non-essential amino acids, 1 mM L-glutamic acid, 0.5 mM sodium pyruvate, and 1× B27 cell culture additive.

Further, the concentration of each component in the serum-free medium is as follows:

| | | |
|---|---|---|
| FGF2 | 30 | ng/ml |
| PDGF-BB | 30 | ng/ml |
| TGF-β 3 | 5 | ng/ml |
| Dexamethasone | 5 | nM |
| Heparin sodium | 2 | μg/ml |
| Vitamin C | 80 | μg/ml |
| Transferrin | 50 | μg/ml |
| Insulin | 0.1 | μg/ml |
| Progesterone | 5 | ng/ml |
| Putrescine | 0.1 | μg/ml |
| Sodium selenite | 5 | ng/ml |
| Epidermal growth factor | 10 | ng/ml |
| CHIR99021 | 2 | μM |
| Non-essential amino acid | 0.1 | mM |
| L-glutamic acid | 1 | mM |
| Sodium pyruvate | 0.5 | mM |

| | |
|---|---|
| B27 Cell Culture Additive | 1X |

### Comparative Example 2

Commercialized Biological Industries MSC Serum-Free Medium (BI SFM):

MSC NutriStem®XF basal medium (medium):MSC nutrient stem Cell®XF supplement (additive):penicillin and streptomycin=500 ml:3 ml:5 ml.

### Comparative Example 3

Commercialized Gibco MSC serum-free culture (ST SFM, or StemPro SFM):StemPro® MSC SFM Supplement CTS™:StemPro® MSC SFM Basal Medium CTS™:L-glutamine or GlutaMAX™-I CTS™=15 ml:84 ml:1 ml, of which the final concentration of L-glutamine or GlutaMAX™-I CTS™ is 2 mM.

### Experimental Example 5 of Biological Activity

Cultured Cell Processing

Tendon stem cells of P3-P6 generation were inoculated into 5 μg/ml laminin-coated well plates, petri dishes or flasks at a density of about 9×10\hat{ }3/cm2, and cultured with serum-free medium, serum control medium, commercial Biological Industries MSC serum-free medium, commercial Gibco MSC serum-free medium. The cells were cultured in a 37° C., 5% CO2 cell incubator, and the medium was changed every 2-3 days. Cell photographs, qPCR and other experiments were performed to observe cell growth and tendon gene expression.

Analysis of Results

Cell Morphology Observation

The method is the same as in Example 1. As shown in FIG. 29, under a 20× microscope, the cell morphology when the cells were basically overgrowth was shown. The results showed that the tendon stem cells cultured in SFM (serum-free medium group) grew vigorously, while the proliferation of the cells in the BI SFM and SCM groups was slower. The cells cultured in ST SFM group has poor cell growth, basically does not proliferate, and produces a lot of cell secretion impurities. Therefore, the cell proliferation of the SFM group was significantly better than that of the SCM (serum medium group) group, the BI SFM group and the ST SFM group, indicating that the serum-free medium is more suitable for tendon stem cells than the two commercial serum-free medium and serum control medium. The commercialized Gibco MSC serum-free culture (ST SFM) is completely unsuitable for the proliferation of tendon stem cells in vitro.

QPCR Detection of Gene Expression in Tendon Lines

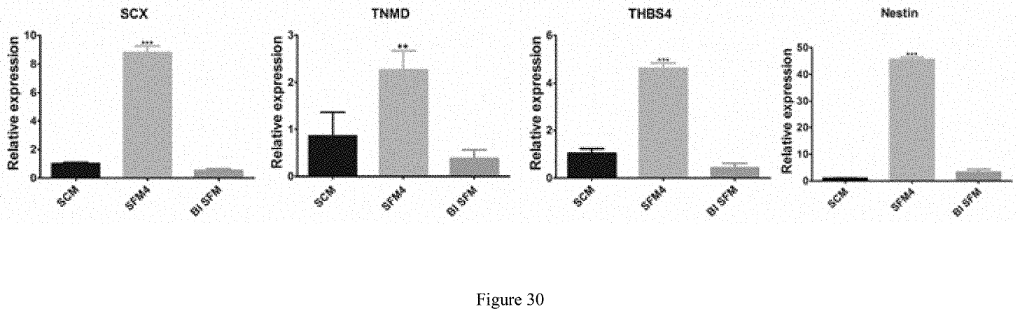

The method is the same as in Example 1. As shown in FIG. 30, serum-free medium (SFM), serum-free medium (SCM), and commercial Biological Industries MSC serum-free medium (BI SFM) are compared. SCX, Nestin, THBS4, TNMD these tendon-related genes were significantly high expressed in the SFM (serum-free medium) group, but the expression in the BI SFM commercial medium was lower, and there was no difference from the serum control group. Therefore, it shows that the serum-free medium is more conducive to the maintenance of tendon lineage phenotype of tendon stem cells than serum control medium and commercial BI MSC serum-free medium.

Flow Cytometry to Detect the Expression of Stem Cell Surface Markers

The method is the same as in Example 1. As shown in Table 2, CD105, CD90, and CD44 are positive expression markers of tendon stem cells, and CD34 and CD18 are negative expression markers of tendon stem cells. The results showed that the positive marker expression of the SFM experimental group was greater than 95%, and the negative marker expression was less than 1%. Moreover, the cells in SFM experimental group were more in line with the characteristics of tendon stem cells than the cells in SCM control group. Therefore, compared with the SCM control, the cells cultured in the serum-free medium are more in line with the features of stem cells.

Clone Forming Ability Test

The method is the same as in Example 1. As shown in Table 3, the results showed that the clone formation ability of the SFM (serum-free medium) experimental group was significantly better than that of the SCM (serum medium) control group. Thus, it shows that compared with serum-free medium, the cells cultured in serum-free medium are more in line with the features of stem cells.

### Example 6

In this example, two groups of culture media were prepared, namely: serum-free medium and serum control culture medium, and the cultured cells were tendon stem cells (Scx-GFP mTSPCs) extracted from the normal tendon tissue of Scx-GFP mice.

The following is the detailed experiment and detection steps:

Medium Configuration

Serum-Free Medium (SFM)

The serum-free medium includes the basal medium and additional components; the basal medium is selected from F12 medium, and 5 mmol of HEPES, 10000 U of penicillin and 10000 U of streptomycin are added to every 500 mL of F12 medium.

Moreover, additional components were added to the medium, and the ratio of the concentration range of each component in the serum-free medium is: fibroblast growth factor:platelet-derived growth factor:transforming growth factor-β:glucocorticoid:heparin or its salt:vitamin C or its derivative:transferrin:insulin:progesterone:putrescine or its salt:selenite=20:20:16:7:2000:4000:2000:10000:10:10000:10. At the same time, the medium also contains 0.01 mM non-essential amino acids, 0.01 mM L-glutamic acid, 0.01 mM sodium pyruvate, and 0.2× B27 cell culture additive. Further, the concentration of each component in the serum-free medium is as follows:

| | | |
|---|---|---|
| FGF-basic | 100 | ng/ml |
| PDGF-AA | 100 | ng/ml |
| TGF-β 1 | 80 | ng/ml |
| Dexamethasone | 90 | nM |
| Heparin sodium | 10 | μg/ml |
| Vitamin C | 20 | μg/ml |
| Transferrin | 10 | μg/ml |
| Insulin | 50 | μg/ml |
| Progesterone | 50 | ng/ml |
| Putrescine | 50 | μg/ml |
| Sodium selenite | 50 | ng/ml |
| Non-essential amino acid | 0.01 | mM |
| L-glutamic acid | 0.01 | mM |
| Sodium pyruvate | 0.01 | mM |

| | |
|---|---|
| B27 Cell Culture Additive | 0.2X |

### Experimental Example 6 of Biological Activity

Cultured Cell Processing

Tendon stem cells of P3-P6 generation were seeded in a low-adhesion 6-well plate at a density of about 5×10\hat{ }4/cm2, and the cells were cultured in serum-free medium and serum control medium, 2 ml per well, 3 replicate wells per group. The cells were cultured in a cell incubator at 37° C. and 5% CO2, the medium was changed every 2-3 days, and experiments such as cell photographing were performed to observe cell growth and tendon gene expression.

Analysis of Results

Cell Morphology Observation

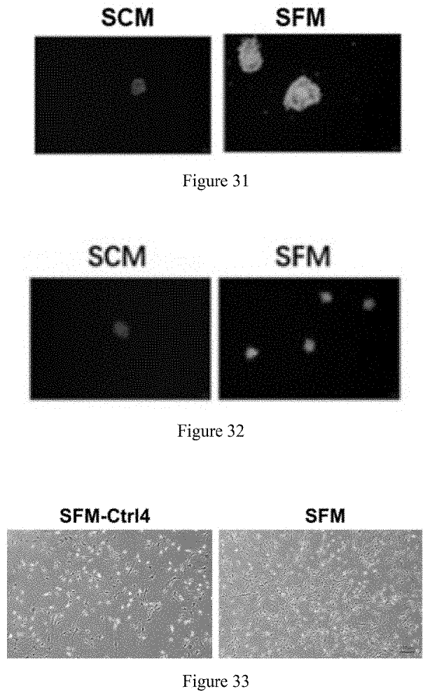

The method is the same as in Example 1. As shown in FIG. 31, under a 10× microscope, the cell morphology when the cells are basically overgrowth is shown. The results showed that the three-dimensional cell spheres formed by Scx-GFPmTSPCs cultured in SFM (serum-free medium group) were larger than those of SCM control group, indicating that the cell proliferation of SFM group was significantly better than that of SCM group. At the same time, SFM (serum-free medium group) cultured Scx-GFP mTSPCs formed three-dimensional cell spheres with stronger GFP fluorescence intensity, indicating that the serum-free medium is more suitable for the maintenance of the SCX phenotype of tendon stem cells than the serum control medium. The results show that our medium also supports the three-dimensional culture of cells.

Flow Cytometry to Detect the Expression of Stem Cell Surface Markers

The method is the same as in Example 1. As shown in Table 2, CD105, CD90, and CD44 are positive expression markers of tendon stem cells, and CD34 and CD18 are negative expression markers of tendon stem cells. The results showed that the positive marker expression of the SFM experimental group was greater than 95%, and the negative marker expression was less than 1%. Moreover, the cells in SFM experimental group were more in line with the characteristics of tendon stem cells than the cells in SCM control group. Therefore, compared with the SCM control, the cells cultured in the serum-free medium are more in line with the features of stem cells.

Clone Forming Ability Test

The method is the same as in Example 1. As shown in Table 3, the results showed that the clone formation ability of the SFM (serum-free medium) experimental group was significantly better than that of the SCM (serum medium) control group. Thus, it shows that compared with serum-free medium, the cells cultured in serum-free medium are more in line with the features of stem cells.

### Example 7

In this example, two groups of culture media were prepared, namely: serum-free medium and serum control culture medium, and the cultured cells were tendon stem cells (Scx-GFP mTSPCs) extracted from the normal tendon tissue of Scx-GFP mice.

The following is the detailed experiment and detection steps:

Medium Configuration

Serum-Free Medium (SFM)

The serum-free medium includes the basal medium and additional components; the basal medium is selected from F12 medium, and 5 mmol of HEPES, 10000 U of penicillin and 10000 U of streptomycin are added to every 500 mL of F12 medium. Moreover, additional components were added to the medium, and the ratio of the concentration range of each component in the serum-free medium is: fibroblast growth factor:platelet-derived growth factor:transforming growth factor-β:glucocorticoid:heparin or its salt:vitamin C or its derivative:transferrin:insulin:progesterone:putrescine or its salt:selenite:epidermal growth factor:CHIR99021=50:50:5:2:5000:100000:5000:5000:5:5000:5:1500:2510. At the same time, the medium also contains 0.01 mM non-essential amino acids, 0.01 mM L-glutamic acid, 0.01 mM sodium pyruvate, and 2× B27 cell culture additive.

Further, the concentration of each component in the serum-free medium is as follows:

| | | |
|---|---|---|
| FGF2 | 1 | ng/ml |
| PDGF-BB | 1 | ng/ml |
| TGF-β 2 | 0.1 | ng/ml |
| Dexamethasone | 0.1 | nM |
| Heparin calcium | 0.1 | μg/ml |
| Vitamin C | 2 | μg/ml |
| Transferrin | 0.1 | μg/ml |
| Insulin | 0.1 | μg/ml |
| Progesterone | 0.1 | ng/ml |
| Putrescine dihydrochloride | 0.1 | μg/ml |
| Sodium selenite | 0.1 | ng/ml |
| Epidermal growth factor | 30 | ng/ml |
| CHIR99021 | 0.1 | μM |
| Non-essential amino acid | 0.01 | mM |
| L-glutamic acid | 0.01 | mM |
| Sodium pyruvate | 0.01 | mM |

| | |
|---|---|
| B27 Cell Culture Additive | 2X |

### Experimental Example 7 of Biological Activity

Cultured Cell Processing

Tendon stem cells of P3-P6 generation were seeded in a low-adhesion 6-well plate at a density of about 1×10\hat{ }5/cm2, and the cells were cultured in serum-free medium and serum control medium, 2 ml per well, 3 replicate wells per group. The cells were cultured in a cell incubator at 37° C. and 5% CO2, the medium was changed every 2-3 days, and experiments such as cell photographing were performed to observe cell growth and tendon gene expression.

Analysis of Results

Cell Morphology Observation

The method is the same as in Example 1. As shown in FIG. 32, under a 10× microscope, the cell morphology when the cells are basically overgrowth is shown. The results showed that the three-dimensional cell spheres formed by Scx-GFPmTSPCs cultured in SFM (serum-free medium group) were larger than those of SCM control group, indicating that the cell proliferation of SFM group was significantly better than that of SCM group. At the same time, SFM (serum-free medium group) cultured Scx-GFP mTSPCs formed three-dimensional cell spheres with stronger GFP fluorescence intensity, indicating that the serum-free medium is more suitable for the maintenance of the SCX phenotype of tendon stem cells than the serum control medium. The results show that our medium also supports the three-dimensional culture of cells.

Flow Cytometry to Detect the Expression of Stem Cell Surface Markers

The method is the same as in Example 1. As shown in Table 2, CD105, CD90, and CD44 are positive expression markers of tendon stem cells, and CD34 and CD18 are negative expression markers of tendon stem cells. The results showed that the positive marker expression of the SFM experimental group was greater than 95%, and the negative marker expression was less than 1%. Moreover, the cells in SFM experimental group were more in line with the characteristics of tendon stem cells than the cells in SCM control group. Therefore, compared with the SCM control, the cells cultured in the serum-free medium are more in line with the features of stem cells.

Clone Forming Ability Test

The method is the same as in Example 1. As shown in Table 3, the results showed that the clone formation ability of the SFM (serum-free medium) experimental group was significantly better than that of the SCM (serum medium) control group. Thus, it shows that compared with serum-free medium, the cells cultured in serum-free medium are more in line with the features of stem cells.

### Example 8

In this example, two groups of culture media were prepared, namely: serum-free medium and serum-free medium for stem cells described in Chinese Patent (CN111206017A). The cultured cells are human mesenchymal stem cells. The following are detailed experiments and detection procedures:

Medium Configuration

The serum-free medium includes a basal medium and additional components; the basal medium is selected from DMEM low-sugar medium, and 1 mmol of HEPES is added to every 500 mL of DMEM low-sugar medium. Moreover, additional components were added to the medium, and the ratio of the concentration range of each component in the serum-free medium is: fibroblast growth factor:platelet-derived growth factor:transforming growth factor-β:glucocorticoid:heparin or its salt:vitamin C or its derivative:transferrin:insulin:progesterone:putrescine or its salt:selenite:epidermal growth factor:CHIR99021=40:15:5:2:3000:20000:50000:100:5:100:5:20:1004. At the same time, the medium also contains 0.1 mM non-essential amino acids, 1 mM L-glutamic acid, 0.5 mM sodium pyruvate, 1× B27 cell culture additive, 3 μg/ml vitronectin synthetic peptide, 3 μg/ml fibronectin synthetic peptide.

Further, the concentration of each component in the serum-free medium is as follows:

| FGF-basic | 40 | ng/ml |
|---|---|---|
| PDGF-BB | 15 | ng/ml |
| TGF-β 3 | 5 | ng/ml |
| Dexamethasone | 5 | nM |
| Heparin sodium | 3 | μg/ml |
| Vitamin C | 20 | μg/ml |
| Transferrin | 50 | μg/ml |
| Insulin | 0.1 | μg/ml |
| Progesterone | 5 | ng/ml |
| Putrescine | 0.1 | μg/ml |
| Sodium selenite | 5 | ng/ml |
| Epidermal growth factor | 20 | ng/ml |
| CHIR99021 | 2 | μM |
| Non-essential amino acid | 0.1 | mM |
| L-glutamic acid | 1 | mM |
| Sodium pyruvate | 0.5 | mM |
| B27 Cell Culture Additive | 1X | |
| Vitronectin synthetic peptide | 3 | μg/ml |
| Fibronectin synthetic peptide | 3 | μg/ml |
| Penicillin (10000 U) | 5 | mmoL |
| Streptomycin (10000 U) | 5 | mmoL |

### Comparative Example 4

Serum-free medium for stem cells described in Chinese Patent (CN111206017A): Add the following components to every 500 mL of DMEM low-sugar basal medium (GIBCO), and make the concentration of the added components in the serum-free medium:

| | | |
|---|---|---|
| Recombinant human serum albumin | 20 | mg/mL |
| Recombinant human PDGF-AA | 20 | ng/mL |
| Recombinant human PDGF-BB | 20 | ng/mL |
| Recombinant human bFGF | 5 | ng/mL |
| Recombinant human TGF-β 1 | 5 | ng/mL |
| Recombinant human EGF | 20 | ng/mL |
| Recombinant IGF | 20 | ng/mL |
| Recombinant human fibronectin | 5 | μg/mL |
| Heparin | 5 | μg/mL |
| Lipid concentrate | 0.1% | (v/v) |
| Recombinant human insulin | 2 | μg/mL |
| Transferrin | 1 | μg/mL |
| Sodium selenite | 1 | ng/mL |
| Galactose | 20 | mM |
| L-glutamine | 292 | mg/L |
| Putrescine | 50 | μM |
| Progesterone | 20 | nM |
| Hydrocortisone | 100 | nM |
| Vitamin C | 200 | μM |
| Vitamin A | 50 | μM |
| Sodium bicarbonate | 20.5 | mM |
| Penicillin (10000 U) | 5 | mmoL |
| Streptomycin (10000 U) | 5 | mmoL |

### Experimental Example 8 of Biological Activity

Cultured Cell Processing

Human mesenchymal stem cells of P3-P6 generation were inoculated into orifice plates or petri dishes at a density of about 5×10\hat{ }3/cm2, cultured in serum-free medium of Example 8 and Comparative Example 4, respectively. The cells were cultured in a cell incubator at 37° C. and 5% CO2. The medium was changed every 2-3 days, and experiments such as cell photographing were performed to observe cell growth and tendon gene expression.

Analysis of Results

Cell Morphology Observation

The method is the same as in Example 1. As shown in FIG. 33, the cell morphology of cells grown for 3 days under the 4× microscope. The results showed that the number of human mesenchymal stem cells cultured in SFM (serum-free medium group) in the same field of view under the same conditions was significantly more than that of comparative example 4 serum-free control group. In addition, the cells in the SFM group had a more uniform morphology, with smaller cells, more transparent and abundant cytoplasm, and better adhesion, indicating that the cell proliferation of the SFM group was significantly better than that of the comparative 4 serum-free control group.

Cell Count and Cell Proliferation Multiple Analysis

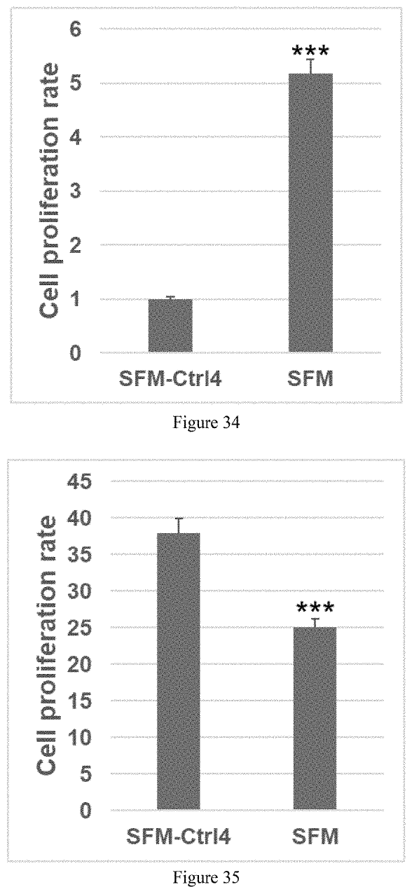

The method is the same as in Example 1. As shown in FIG. 34, the number of cells obtained in the serum-free control group of Comparative Example 4 is 1. The proliferation rate of human mesenchymal stem cells in the medium) group was significantly higher than that of the control group without serum.

Cell Doubling Time Analysis

The method is the same as in Example 1. As shown in FIG. 35, the doubling time of human mesenchymal stem cells in the SFM (serum-free medium) of Example 8 was significantly lower than that of SFM-Ctrl4 (comparative example 4 serum-free control group). The doubling time of the SFM-Ctrl4 group was 1.52 times that of the SFM group. The shorter the doubling time, the faster the cell proliferation, that is, the proliferation rate of human mesenchymal stem cells in SFM group was significantly faster than that of SFM-Ctrl4. This indicates that the serum-free medium composed of biologically active substances of the present invention has a significantly better ability to promote cell proliferation than the stem cell serum-free medium described in the Chinese patent (202010104684. 5).

Flow Cytometry to Detect the Expression of Stem Cell Surface Markers

The method is the same as in Example 1. As shown in Table 2, CD105, CD90, and CD44 are positive expression markers of human mesenchymal stem cells, and CD34 and CD18 are negative expression markers of human mesenchymal stem cells. The results showed that the expression of positive markers of SFM in the two groups was greater than 95%. The negative marker expression of human mesenchymal stem cells cultured in the SFM (serum-free medium) of Example 8 was less than 1%, and the negative marker expression of the cells cultured with SFM-Ctrl4 (comparative example 4 serum-free control group) was greater than 1%. It shows that the characteristics of the serum-free medium for culturing cell stem cells composed of biologically active substances of the present invention are slightly better than those of the serum-free medium for stem cells described in the Chinese patent (202010104684. 5).

Clone Forming Ability Test

The method is the same as in Example 1. As shown in Table 3, the results showed that the clone formation ability of the SFM (serum-free medium) experimental group of Example 8 was significantly better than that of the SFM-Ctrl4 (comparative example 4 serum-free control group). This shows that the characteristics of the serum-free medium composed of biologically active substances of the present invention for culturing cell stem cells are better than those described in the Chinese patent (202010104684. 5).

In summary, the Chinese patent (CN111206017A) discloses a stem cell serum-free medium and its application. From the experimental data provided by the invention patent and the comparative experiment conducted by the invention patent, it is found that the evaluation from the multi-angles of cell morphology, cell proliferation rate and cell doubling time shows that the proliferation rate of stem cells cultivated in this patent is significantly slower than that of the serum-free medium prepared by the bioactive substance composition of the patent of the present invention (FIGS. 32-34). Moreover, the patent does not provide sufficient evidence to prove that its published medium can be used for primary culture. It does not prove the safety of its cultured cells, and there is no in vivo animal experiment to prove that its cultured cells can be used for tissue engineering and injury repair. It cannot prove that its cultured cells can be used for clinical cell therapy.

### Example 9

In this example, a serum-free medium was prepared, and the cultured cells were human chondrocytes. The following are detailed experiments and detection steps:

Medium Configuration

The serum-free medium includes a basal medium and additional components; the basal medium is selected from DMEM low-sugar medium, and 1 mmol of HEPES is added to every 500 mL of DMEM low-sugar medium. Moreover, additional components were added to the medium, and the ratio of the concentration range of each component in the serum-free medium is: fibroblast growth factor:platelet-derived growth factor:transforming growth factor-β:glucocorticoid:heparin or its salt:vitamin C or its derivative:transferrin:insulin:progesterone:putrescine or its salt:selenite:epidermal growth factor:CHIR99021=40:40:3:2:5000:10000:1000:10:1:2:1:20:1004. At the same time, the medium also contains 0.1 mM non-essential amino acids, 1 mM L-glutamic acid, 0.5 mM sodium pyruvate, and 1× B27 cell culture additive.

Further, the concentration of each component in the serum-free medium is as follows:

| FGF2 synthetic peptide | 40 | ng/ml |
|---|---|---|
| PDGF-BB synthetic peptide | 40 | ng/ml |
| TGF-β 3 synthetic peptide | 3 | ng/ml |
| Dexamethasone | 5 | nM |
| Heparin sodium | 5 | μg/ml |
| Vitamin C | 10 | μg/ml |
| Transferrin | 1 | μg/ml |
| Insulin | 0.01 | μg/ml |
| Progesterone | 1 | ng/ml |
| Putrescine | 2 | ng/ml |
| Sodium selenite | 1 | ng/ml |
| Epidermal growth factor synthetic peptide | 20 | ng/ml |
| CHIR99021 | 2 | μM |
| Non-essential amino acid | 0.1 | mM |
| L-glutamic acid | 1 | mM |
| Sodium pyruvate | 0.5 | mM |
| B27 Cell Culture Additive | 1X | |
| Penicillin (10000 U) | 5 | mmoL |
| Streptomycin (10000 U) | 5 | mmoL |

### Experimental Example 9 of Biological Activity

Cultured Cell Processing

Human chondrocytes of P3-P6 generation were inoculated into a common 10 cm petri dish at a density of about 1×10\hat{ }4/cm2 in a serum-free medium in a 37° C., 5% CO2 cell incubator. The medium was changed every 2-3 days, and the cells were subjected to cell photography and other experiments to observe the cell growth.

Analysis of Results

Cell Morphology Observation

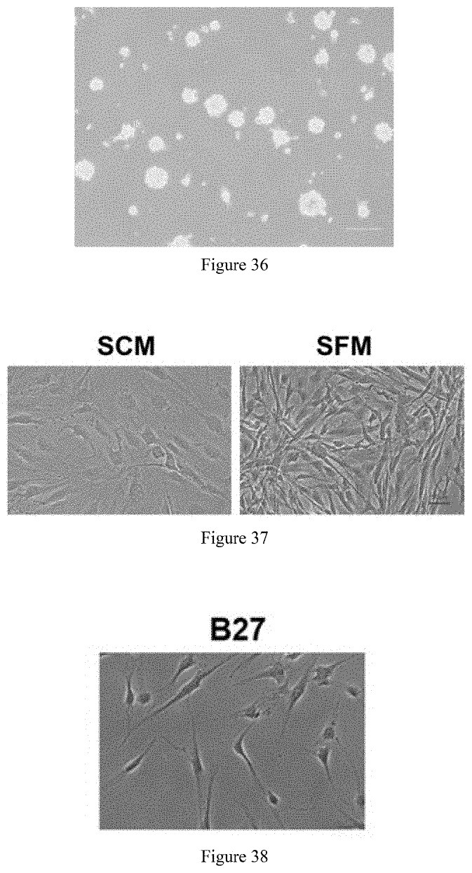

The method is the same as in Example 1. As shown in FIG. 36, 4× microscope shows that human chondrocytes cultured in SFM (serum-free medium group) can form large three-dimensional cell spheres well. In addition, the diameter of the spheres showed a tendency to increase, indicating that the serum-free medium is suitable for the culture of chondrocytes. At the same time, this result shows that our medium also supports the three-dimensional culture of cells.

Cell Count and Cell Proliferation Multiple Analysis

The method is the same as in Example 1. The cell count results showed that the number of harvested cells was 4.29×10\hat{ }6, and the total amount of initial cell inoculation was 5.5×10\hat{ }5. Therefore, the cell proliferation was 7.8 times. This result shows that the serum-free medium composed of biologically active substances of the present invention is suitable for chondrocytes in vitro expansion culture.

### Example 10

In this example, two groups of culture media were prepared, namely a serum-free medium and a serum control culture medium. The cultured cells were human skeletal stem cells. The following are detailed experiments and detection procedures:

Medium Configuration

The serum-free medium includes a basal medium and additional components; the basal medium is selected from BEM medium, and 1 mmol of HEPES is added to every 500 mL of BEM medium. Moreover, additional components were added to the medium, and the ratio of the concentration range of each component in the serum-free medium is: fibroblast growth factor synthetic peptide:platelet-derived growth factor synthetic peptide:transforming growth factor-β synthetic peptide:glucocorticoid:heparin or its salt:vitamin C or its derivatives:transferrin:insulin:progesterone:putrescine or its salt:selenite:epidermal growth factor synthetic peptide:CHIR99021=30:30:20:5:2000:80,000:80,000:5000:7:10000:7:20:1004. At the same time, the medium also contains 0.1 mM non-essential amino acids, 1 mM L-glutamic acid, 0.5 mM sodium pyruvate, 1× B27 cell culture additive, 0.1 μg/ml vitronectin synthetic peptide, 0.1 μg/ml fibronectin synthetic peptide, 0.1 μg/ml laminin synthetic peptide.

Further, the concentration of each component in the serum-free medium is as follows:

| FGF-basic | 20 | ng/ml |
|---|---|---|
| FGF1 synthetic peptide | 10 | ng/ml |
| PDGF-AA | 20 | ng/ml |
| PDGF-AB synthetic peptide | 10 | ng/ml |
| TGF-β3 | 10 | ng/ml |
| TGF-β 1 synthetic peptide | 10 | ng/ml |
| Dexamethasone | 12.5 | nM |
| Heparin sodium | 2 | μg/ml |
| Vitamin C | 80 | μg/ml |
| Transferrin | 80 | μg/ml |
| Insulin | 5 | μg/ml |
| Progesterone | 7 | ng/ml |
| Putrescine | 10 | μg/ml |
| Sodium selenite | 7 | ng/ml |
| Epidermal growth factor | 20 | ng/ml |
| CHIR99021 | 2 | μM |
| Non-essential amino acid | 0.1 | nM |
| L-glutamine | 1 | mM |
| Sodium pyruvate | 0.5 | mM |
| B27 Cell Culture Additive | 1X | |
| Vitronectin synthetic peptide | 0.1 | μg/ml |
| Fibronectin synthetic peptide | 0.1 | μg/ml |
| Laminin synthetic peptide | 0.1 | μg/ml |
| Penicillin (10000 U) | 5 | mmol |
| Streptomycin (10000 U) | 5 | mmoL |

### Experimental Example 10 of Biological Activity

Cultured Cell Processing

Human skeletal stem cells of P3-P6 generation were seeded into 500 mg/ml RGD (Arg-Gly-Asp) peptide and 200 mg/ml KRSR (Lys-Arg-Ser-Arg) peptide coated 10 cm petri dish. The cells were cultured in a serum-free medium in a cell incubator at 37° C. and 5% CO2, and the medium was changed every 2-3 days. Experiments such as taking pictures of the cells were performed to observe the growth of the cells.

Analysis of Results

Cell Morphology Observation

The method is the same as in Example 1. As shown in FIG. 37, observation under the 20× microscope showed that the human bone stem cells cultured in SFM (serum-free medium) grew vigorously, and the cell proliferation was significantly better than that of the SCM (serum medium) group. In addition, the human skeletal stem cells cultured in SFM (serum-free medium) had a more uniform cell morphology and size, a transparent and abundant cytoplasm, and a good adherence to the wall. This result indicates that the composition composed of biologically active substances of the present invention is suitable for the expansion and culture of bone stem cells in vitro.

Clone Forming Ability Test

The method is the same as in Example 1. As shown in Table 3, the results show that the clone formation ability of the SFM (serum-free medium) experimental group of Example 10 is significantly better than that of the SCM serum control group. Therefore, it is shown that the features of the stem cells of the skeletal stem cells cultured in the serum-free medium composed of the biologically active substance of the present invention are better than those of the skeletal stem cells cultured in the serum control group.

### Comparative Example 5

Medium Containing Only B27 Cell Culture Additives (B27)

The medium containing only B27 cell culture additives was selected from DMEM/F12 medium, and 5 mmol of HEPES, 10000 U of penicillin and 10000 U of streptomycin were added to every 500 mL of DMEM/F12 medium. In addition, the additional components were added, and the concentration of the additional components in the serum-free medium were:

| | | |
|---|---|---|
| Non-essential amino acid | 0.1 | mM |
| L-glutamic acid | 2 | mM |
| Sodium pyruvate | 1 | mM |

| | |
|---|---|
| B27 Cell Culture Additive | 1X |

### Experimental Example 5 of Biological Activity

Cultured Cell Processing

Tendon stem cells of P3-P6 generation were inoculated into a 12-well plate coated with 20 mg/ml type I collagen at a density of about 9×10\hat{ }3/cm2. Culture the cells with serum-free medium, B27 control medium and serum control medium. 1 ml per well, 3 multiple wells per group. Place the cells in a 37° C., 5% CO2 cell incubator, change the medium every 3 days, culture for 5 days, and take photos of the cells to observe the cell growth.

Analysis of Results

Cell Morphology Observation

The method is the same as in Example 1. As shown in FIG. 38, under a 20× microscope, the growth of the cells after 5 days of culture was displayed. The results showed that the cells in group B27 did not proliferate, indicating that the cell culture additives alone cannot effectively proliferate the cells.

### Comparative Example 6

In this comparative example, a set of serum-free media disclosed in the paper (Chinese Journal of Experimental Surgery. 2014. 31(2): 395-398) was configured, and the cultured cells were tendon stem cells (hTSPCs) extracted from human normal tendon tissue. The following is the detailed experiment and detection steps:

Medium Configuration

Comparative Serum-Free Medium

The serum-free medium includes a basal medium and additional components; the basal medium is selected from α-MEM medium, and every 500 mL of α-MEM medium is added with 25 μg IGF-1 and 5 ug TGF-β3, namely 50 μg/L IGF-1 and 10 μg/L TGF-β3.

### Comparative Example 6 of Biological Activity

Cultured Cell Processing

The primary culture of the cultured cells was carried out with serum culture medium.

After passage, culture in the comparative medium was carried out. Other methods are the same as in Example 1.

Analysis of Results

Cell Morphology Observation

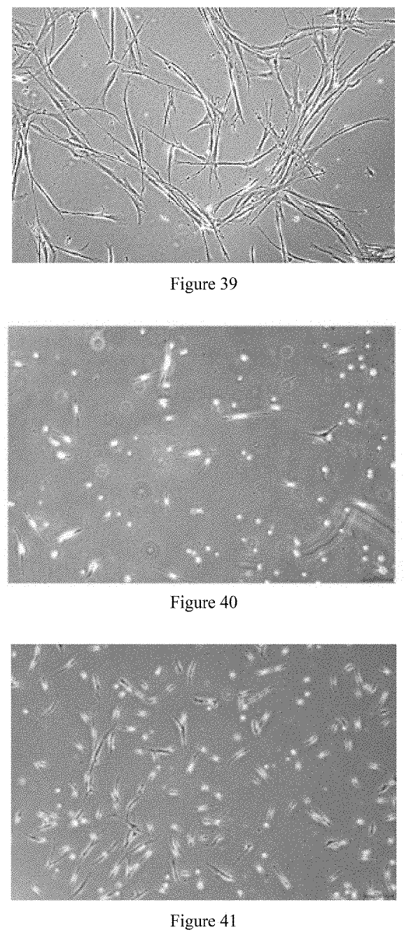

The method is the same as in Example 1. As shown in FIG. 39, the growth of the cells after 5 days of culture under the 4× microscope showed that the tendon stem cells cultured in the serum-free medium of the comparative example showed slow proliferation. This result indicates that the medium of this paper cannot maintain cell proliferation. At the same time, since the cultured cells were derived from the serum medium, there was serum residue, and the safety was 0 points. Therefore, this comparative example shows that the primary culture is a serum culture medium, and the subsequent secondary culture is a serum-free culture. The cells obtained cannot meet the requirements of the number and quality of clinical treatment cells.

### Comparative Example 7

In this comparative example, a set of serum-free medium with a concentration far beyond the concentration range of the existing biologically active substances was configured, and the cultured cells were tendon stem cells (hTSPCs) extracted from human normal tendon tissue. The following are detailed experiments and detection procedures:

Medium Configuration

Comparative Serum-Free Medium

The serum-free medium includes a basal medium and additional components; the basal medium is selected from DMEM/F12 medium, and every 500 mL of DMEM/F12 medium was supplemented with 5 mmol of HEPES, 10,000 U of penicillin, and 10,000 U of streptomycin. In addition, the additional components were added, and the concentration of the additional components in the serum-free medium were: fibroblast growth factor:platelet-derived growth factor:transforming growth factor-β:glucocorticoid:heparin or its salt:vitamin C or its derivative:transferrin:insulin:progesterone:putrescine or its salt:selenite=203:120:100:120:13000:200000:400,000:100000:100:100000:100. At the same time, the medium also contains 0.1 mM non-essential amino acids, 2 mM L-glutamic acid, 1 mM sodium pyruvate, and 6× B27 cell culture additives.

| | | |
|---|---|---|
| FGF-basic | 203 | ng/ml |
| PDGF-AA | 120 | ng/ml |
| TGF-β 1 | 100 | ng/ml |
| Dexamethasone | 300 | nM |
| Heparin sodium | 200 | μg/ml |
| Transferrin | 400 | μg/ml |
| Insulin | 100 | μg/ml |
| Progesterone | 100 | ng/ml |
| Putrescine | 100 | μg/ml |
| Sodium selenite | 100 | ng/ml |
| Non-essential amino acid | 0.1 | mM |
| L-glutamic acid | 2 | mM |
| Sodium pyruvate | 1 | mM |

| | |
|---|---|
| B27 Cell Culture Additive | 6X |

Comparative Example 6 of Biological Activity

Cultured Cell Processing

Tendon stem cells of P3-P6 generation were inoculated into a 12-well plate coated with 20 mg/ml type I collagen at a density of about 9×10\hat{ }3/cm2. Serum-free medium, B27 control medium and serum control were used respectively. Cultivate the cells, 1 ml per well, 3 replicate wells per group, and place them in a 37° C., 5% CO2 cell incubator. Change the medium every 3 days for 5 days, and take photos of the cells to observe the cell growth.

Analysis of Results

Cell Morphology Observation

The method is the same as in Example 40. As shown in FIG. 39, the growth of the cells after 5 days of culture was displayed under a 4× microscope. The results showed that the tendon stem cell cells cultured in the serum-free medium of the comparative example did not proliferate or even died. The concentration range of each component of the medium is unique.

### Comparative Example 8

The medium prepared in this comparative example was not added with fibroblast growth factor, and other conditions were the same as in Example 2.

Analysis of Results

Cell Morphology Observation

The method is the same as in Example 1. As shown in FIG. 41, the growth of the cells after 5 days of culture was shown under a 4× microscope. The results showed that the cultured tendon stem cells in the serum-free medium of this comparative example did not proliferate. This result indicates that the components of the biologically active composition and serum-free medium developed by the present invention are necessary for its function, and the uniqueness of each component of the bioactive substance composition of the present invention.

### Comparative Example 9

The medium prepared in this comparative example did not add transforming growth factor-β, but added 5 ng/ml epidermal growth factor, and other conditions were the same as in Example 2.

Analysis of Results

The cell culture effect of the comparative example shows that cell proliferation is slowed down and the phenotype maintenance ability is significantly reduced, indicating that the components of the biologically active composition developed by the present invention are necessary for its function and cannot be replaced by other components, indicating the uniqueness of each component of the bioactive substance composition of the present invention.

### Example 11

A set of serum-free medium was prepared in this example and the cultured cells were tendon stem cells (Scx-GFP mTSPCs) extracted from the normal tendon tissue of Scx-GFP mice. The following are the detailed experiments and detection steps:

Medium Configuration

The serum-free medium includes a basal medium and additional components; the basal medium is selected from DMEM/F12 medium, and every 500 mL of DMEM/F12 medium was supplemented with 5 mmol of HEPES, 10,000 U of penicillin, and 10,000 U of streptomycin. In addition, the additional components were added, and the concentration of the additional components in the serum-free medium were: fibroblast growth factor:platelet-derived growth factor:transforming growth factor-β:glucocorticoid:heparin or its salt:vitamin C or its derivative:transferrin:insulin:progesterone:putrescine or its salt:selenite=5:5:2:1:4000:90000:200000:15000:15:15000:15. At the same time, the medium also contains 0.1 mM non-essential amino acids, 1 mM L-glutamine, 0.5 mM sodium pyruvate, 1×B27 cell culture supplement.

Further, the concentration of each component in the serum-free medium is as follows:

| FGF-basic | 5 | ng/ml |
|---|---|---|
| PDGF-AA | 5 | ng/ml |
| TGF-β3 | 2 | ng/ml |
| Dexamethasone | 2.5 | nM |
| Heparin sodium | 4 | μg/ml |
| Vitamin C | 90 | μg/ml |
| Transferrin | 200 | μg/ml |
| Insulin | 15 | μg/ml |
| Progesterone | 15 | ng/ml |
| Putrescine | 15 | μg/ml |
| Sodium selenite | 15 | ng/ml |
| Non-essential amino acid | 0.1 | mM |
| L-glutamine | 1 | mM |
| Sodium pyruvate | 0.5 | mM |
| B27 Cell Culture Additive | 1X | |
| Penicillin (10000 U) | 5 | mmoL |
| Streptomycin (10000 U) | 5 | mmoL |

### Experimental Example 11 of Biological Activity

The treatment of cultured cells was the same as in Example 6.

Analysis of Results

The cell culture effect of this example was similar to that of Example 6. It shows that the culture medium of the present invention supports the culture of mouse tendon stem cells and also supports cell suspension culture.

### Example 12

In this embodiment, a set of serum-free medium was prepared, and the cultured cells were ligament stem cells obtained by separation and culture of human ligament tissue.

The following are detailed experiments and detection steps:

Medium Configuration

Serum-Free Medium (SFM)

The serum-free medium includes a basal medium and additional components; the basal medium is selected from F10 medium, and every 500 mL of F10 medium was supplemented with 5 mmol of HEPES, 10,000 U of penicillin, and 10,000 U of streptomycin. In addition, the additional components were added, and the concentration of the additional components in the serum-free medium were: fibroblast growth factor:platelet-derived growth factor:transforming growth factor-β:glucocorticoid:heparin or its salt:vitamin C or its derivative:transferrin:insulin:progesterone:putrescine or its salt:selenite=26:15:30:8:500:1000:75000:3000:4:7:4. At the same time, the medium also contains 0.1 mM non-essential amino acids, 2 mM L-glutamic acid, 1 mM sodium pyruvate, 1× B27 cell culture additive, and 2 μg/ml vitronectin.

Further, the concentration of each component in the serum-free medium is as follows:

| FGF-basic | 26 | ng/ml |
|---|---|---|
| PDGF-BB | 15 | ng/ml |
| TGF-β 2 | 30 | ng/ml |
| Dexamethasone | 20 | nM |
| Heparin sodium | 0.5 | μg/ml |
| Vitamin C | 1 | μg/ml |
| Transferrin | 75 | μg/ml |
| Insulin | 3 | μg/ml |
| Progesterone | 4 | ng/ml |
| Putrescine | 7 | μg/ml |
| Sodium selenite | 4 | ng/ml |
| Non-essential amino acid | 0.1 | mM |
| L-glutamic acid | 2 | mM |
| Sodium pyruvate | 1 | mM |
| B27 Cell Culture Additive | 1X | |
| Vitronectin | 2 | ug/ml |
| Penicillin (10000 U) | 5 | mmoL |
| Streptomycin (10000 U) | 5 | mmoL |

### Experimental Example 12 of Biological Activity

The treatment of cultured cells is the same as in Example 2

Analysis of Results

The cell culture effect of this example is similar to that of example 2. It shows that the culture medium of the present invention supports the cultivation of human ligament stem cells.

TABLE 1
The viability of cultured cells in different
examples and comparative examples

| | Cell viability |
|---|---|
| Example 1 | 98% |
| Example 2 | 95.4% |
| Example 3 | 90.3% |
| Example 4 | 92% |
| Example 5 | 97.6% |
| Example 6 | 88% |
| Example 7 | 91% |
| Example 8 | 95.4% |
| Example 9 | 92% |
| Example 10 | 94.5% |
| Example 11 | 90.6% |
| Example 12 | 93% |
| Comparative | 78% |
| Example 1 | |
| Comparative | 90% |
| Example 2 | |
| Comparative | 65% |
| Example 3 | |
| Comparative | 87.4% |
| Example 4 | |
| Comparative | 40% |
| Example 5 | |
| Comparative | 72% |
| Example 6 | |
| Comparative | 0 |
| Example 7 | |
| Comparative | 71% |
| Example 8 | |
| Comparative | 69.5% |
| Example 9 | |

TABLE 2
The results of flow cytometry indicate the expression
of CD markers on the surface of cells cultured
in different examples and comparative examples

| | CD105 | CD90 | CD44 | CD34 | CD18 |
|---|---|---|---|---|---|
| Example 1 | 98.9% | 99.9% | 99.9% | 0.4% | 0.1% |
| Example 2 | 98.7% | 99.8% | 99.5% | 0.3% | 0.3% |
| Example 3 | 98.5% | 99.5% | 99.4% | 0.1% | 0.3% |
| Example 4 | 99.3% | 99.8% | 99.7% | 0.5% | 0.4% |
| Example 5 | 99.5% | 99.7% | 99.2% | 0.3% | 0.1% |
| Example 6 | 98.1% | 97.3% | 97.5% | 0.6% | 0.6% |
| Example 7 | 98.3% | 96.8% | 97.2% | 0.8% | 1% |
| Example 8 | 99.8% | 99.2% | 98.6% | 0.8% | 0.6% |
| Example 11 | 97.9% | 98.2% | 97.4% | 0.2% | 0.5% |
| Example 12 | 98.9% | 98.8% | 99.2% | 0.7% | 0.9% |
| Comparative | 97.2% | 95.5% | 96.5% | 2.8% | 1% |
| Example 1 | | | | | |
| Comparative | 95.5% | 92.2% | 96.3% | 2.4% | 2% |
| Example 2 | | | | | |
| Comparative | \ | \ | \ | \ | \ |
| Example 3 | | | | | |
| Comparative | 99.6% | 98.7% | 95.4% | 1.1% | 1.6% |
| Example 4 | | | | | |
| Comparative | \ | \ | \ | \ | \ |
| Example 5 | | | | | |
| Comparative | \ | \ | \ | \ | \ |
| Example 6 | | | | | |
| Comparative | \ | \ | \ | \ | \ |
| Example 7 | | | | | |
| Comparative | \ | \ | \ | \ | \ |
| Example 8 | | | | | |
| Comparative | \ | \ | \ | \ | \ |
| Example 9 | | | | | |

Note:
\ Means that the cells did not proliferate in this medium, and the amount of cells is too small to be detected.

TABLE 3
CFU experiment results show the clone formation
ability of P3 generation cell tendon line in
different examples and comparative examples.

| | Number of single clones |
|---|---|
| | formed in each well/piece |
| Example 1 | 25 |
| Example 2 | 19 |
| Example 3 | 20 |
| Example 4 | 17 |
| Example 5 | 23 |
| Example 6 | 15 |
| Example 7 | 13 |
| Example 8 | 23 |
| Example 10 | 21 |
| Example 11 | 16 |
| Example 12 | 20 |
| Comparative | 12 |
| Example 1 | |
| Comparative | 6 |
| Example 2 | |
| Comparative | 0 |
| Example 3 | |
| Comparative | 12 |
| Example 4 | |
| Comparative | 0 |
| Example 5 | |
| Comparative | 0 |
| Example 6 | |
| Comparative | 0 |
| Example 7 | |
| Comparative | 0 |
| Example 8 | |
| Comparative | 4 |
| Example 9 | |

TABLE 4
Percentage of Nestin+ Cells Cultured
in Each Example and Comparative Example

| | Nestin + TSPC(%) |
|---|---|
| Example 1 | 94 |
| Example 2 | 90 |
| Example 3 | 78 |
| Example 4 | 72 |
| Example 5 | 92 |
| Example 6 | 35 |
| Example 7 | 60 |
| Example 8 | 92 |
| Example 10 | 86 |
| Example 11 | 62 |
| Example 12 | 71 |
| Comparative | 5 |
| Example 1 | |
| Comparative | 3 |
| Example 2 | |
| Comparative | 0 |
| Example 3 | |
| Comparative | 5 |
| Example 4 | |
| Comparative | 0 |
| Example 5 | |
| Comparative | 10 |
| Example 6 | |
| Comparative | 0 |
| Example 7 | |
| Comparative | 5 |
| Example 8 | |
| Comparative | 2 |
| Example 9 | |

TABLE 5
Scoring of cells cultured in each example and comparative example

| | | | | 4. Tendon | | |
|---|---|---|---|---|---|---|
| | | | | phenotype | 5. The in vivo | |
| | | | | and tendon | ability to | |
| | 1. Proliferation | 2. Stem cell | | differentiation | repair tendon | Total |
| | rate | phenotype | 3. Security | ability | injuries | score |
| Example 1 | 30 | 20 | 10 | 20 | 20 | 100 |
| Example 2 | 20 | 20 | 10 | 20 | 20 | 90 |
| Example 3 | 25 | 15 | 10 | 15 | 20 | 85 |
| Example 4 | 20 | 15 | 10 | 15 | 20 | 80 |
| Example 5 | 30 | 20 | 10 | 20 | 20 | 100 |
| Example 6 | 15 | 15 | 10 | 10 | 15 | 65 |
| Example 7 | 10 | 10 | 10 | 15 | 15 | 60 |
| Example 8 | 30 | 20 | 10 | 20 | 20 | 100 |
| Example 10 | 30 | 20 | 10 | 20 | 0 | 80 |
| Example 11 | 15 | 15 | 10 | 15 | 15 | 70 |
| Example 12 | 20 | 20 | 10 | 15 | 20 | 85 |
| Comparative | 5 | 10 | 0 | 0 | 0 | 15 |
| Example 1 | | | | | | |
| Comparative | 25 | 10 | 10 | 0 | 0 | 45 |
| Example 2 | | | | | | |
| Comparative | 0 | 0 | 10 | 0 | 0 | 10 |
| Example 3 | | | | | | |
| Comparative | 25 | 15 | 0 | 0 | 0 | 40 |
| Example 4 | | | | | | |
| Comparative | 0 | 0 | 10 | 0 | 0 | 10 |
| Example 5 | | | | | | |
| Comparative | 0 | 0 | 0 | 5 | 0 | 5 |
| Example 6 | | | | | | |
| Comparative | 0 | 0 | 10 | 0 | 0 | 10 |
| Example 7 | | | | | | |
| Comparative | 0 | 0 | 10 | 0 | 0 | 10 |
| Example 8 | | | | | | |
| Comparative | 5 | 0 | 10 | 0 | 0 | 15 |
| Example 9 | | | | | | |

The comparison of each example with Comparative Example 1 shows that the serum-free medium and/or composition described in this application is a completely serum-free medium, which can completely replace the serum-containing medium, realize the primary culture and secondary culture of cells, and realize the rapid proliferation of cells in vitro and the maintenance or improvement of phenotype.

The comparison between Example 1 and Comparative Example 1 shows that the cells cultured in the serum-free medium and/or composition described in this application proliferate rapidly and the phenotype is significantly increased. These cells cultured in vitro with good proliferation and phenotype are still powerful when transplanted into the body, and can realize tissue injury repair. The tissue injury repair effect of the cell in Example 1 is significantly better than that of Comparative Example 1 cells obtained from the control group cultured in serum-containing medium. It shows that the cells obtained by the serum-free medium and/or composition culture of the present application have strong functions, and can quickly participate in the regeneration of injured parts and repair tissue injury when implanted in the body. The tissue or organ injury is selected from musculoskeletal system tissue or organ injury; preferably, the musculoskeletal system tissue or organ injury is selected from at least one of tendon and/or ligament injury, cartilage injury, bone injury, muscle injury, skin injury, blood vessel injury.

The cells cultured in Examples 6, 7, and 11 are of animal origin, and the cells cultured in other examples are of human origin, indicating that the serum-free medium and/or composition described in this application can achieve in vitro culture of cells of human or animal origin. At the same time, Examples 6 and 7 are three-dimensional suspension culture, and other examples are adherent culture, indicating that the serum-free medium and/or composition described in this application can not only carry out suspension culture of cells, but also carry out adherent culture of cells.

The results of comparison between Example 5 and Comparative Examples 2 and 3 (common MSC commercial serum-free medium) show that the cell proliferation ability, stem cell phenotype and tendinoid phenotype of the serum-free medium and/or composition described in this application are significantly better than that of Comparative Examples 2 and 3. It shows that the serum-free medium and/or composition described in this application are more suitable for in vitro culture of cells and maintenance/improvement of phenotype.

The experimental results of Comparative Example 7 show that the use concentration range of the biologically active substance and the serum-free medium developed by the present invention is unique, and the concentration range beyond the concentration range is not effective. The experimental results of Comparative Examples 8 and 9 indicate that the biologically active composition and the serum-free medium developed by the present invention are necessary for their functions and cannot be replaced. It illustrates the uniqueness of the composition and concentration of each component of the bioactive substance composition of the present invention.

In summary, this application adjusts the serum-free medium used in the process of in vitro expansion of stem cells by adjusting the basal medium, bioactive substance composition, additives and their content, and/or the bioactive substance composition of the composition. The additives and their contents are verified using different cell cultures, and a serum-free medium and/or composition that can improve the proliferation ability and phenotype of cells in vitro is obtained. The cells are selected from any one or more of cells derived from tendons and/or ligaments, mesenchymal stem cells, meniscal stem cells, chondrocytes, skeletal stem cells, and muscle stem cells. Preferably, the cell common features and cell-specific phenotypes of the cells cultured in the serum-free medium and/or the composition have individual scores of each individual item reaching their respective passing lines and the total score reaching 60 points or more. Preferably, the cell common features and cell-specific phenotypes of the cells cultured in the serum-free medium reach the respective passing line and the total score reaches 80 points or more. Preferably, the cell common features and the cell-specific phenotype of the cells cultured in the serum-free medium have a single score of each individual item reaching their respective passing line and a total score of 90 points or more.

It can be seen from the various examples and comparative examples that the serum-free medium and/or composition described in this application can achieve in vitro expansion and phenotype maintenance of tendon and/or ligament-derived cells, mesenchymal stem cells, meniscus stem cells, chondrocytes, skeletal stem cells, and muscle stem cells. These cells are the main cell members of the musculoskeletal system and play an important role in the formation and function of the musculoskeletal system.

For example, tendon is composed of two major components: tendon-derived cells and collagen matrix. Tendon-derived cells are the only cell member of tendon tissue and play a major role in the development, homeostasis maintenance, and injury repair of tendon tissue, and the collagen matrix in tendon is also formed by the secretion of tendon-derived cells. The serum-free medium and/or composition described in the present application realize the in vitro proliferation and phenotype maintenance of tendon stem cells, and therefore, the in vitro culture and functional maintenance of tendon tissue can also be realized. Therefore, the serum-free medium and/or composition described in this application can be used for in vitro culture and functional maintenance of tissues derived from the exercise system. Preferably, the exercise system tissue is selected from tendon tissue, ligament tissue, meniscus tissue, cartilage tissue, adipose tissue, muscle tissue. At the same time, it can be seen from the various examples that the serum-free medium and composition can promote the in vitro expansion of cells and the maintenance or improvement of phenotype, and the core components of these two substances are biologically active substances. It also shows that the bioactive substance composition has super activity and can be used to prepare cell culture reagents. The bioactive substance composition, the serum-free medium, and the composition utilize each component to simulate the complex microenvironment of cell growth in vivo to realize cell culture in vitro. In the process of tissue injury, the microenvironment of the tissue injury site is destroyed, resulting in slow tissue regeneration/repair. Injecting and/or smearing the bioactive substance composition and/or serum-free medium and/or the composition at the injury site can rapidly remodel the microenvironment of the injury site in the body, and accelerate injury repair and tissue regeneration. Therefore, the bioactive substance composition and/or serum-free medium and/or composition described in the present application have application in the preparation of a medicine for the treatment of tissue and/or organ injury. The tissue or organ injury is selected from musculoskeletal system tissue or organ injury; preferably, the musculoskeletal system tissue or organ injury is selected from at least one of tendon and/or ligament injury, cartilage injury, bone injury, muscle injury, skin injury, blood vessel injury.

What is claimed is:

1. A bioactive substance composition, wherein the bioactive substance composition comprises fibroblast growth factor, platelet-derived growth factor, transforming growth factor-β, glucocorticoid, heparin or its salt, vitamin C or its derivatives, transferrin, insulin, progesterone, putrescine or its salt, selenite, wherein the mass-volume concentration range ratio of each component is:
fibroblast growth factor:platelet-derived growth factor:transforming growth factor-β:glucocorticoid:heparin or its salt:vitamin C or its derivative:transferrin:insulin:progesterone:putrescine or its salt:selenite=1-50:1-50:1-40:1-11:10-5000:10-100000:10-300000:1-25000:1-25:1-25000:1-25;
preferably, fibroblast growth factor:platelet-derived growth factor:transforming growth factor-β:glucocorticoid:heparin or its salt:vitamin C or its derivative:transferrin:insulin:progesterone:putrescine or its salt:selenite=5-40:5-40:2-30:1-8:500-4000:1000-90000:1000-200000:10-15000:1-15:2-15000:1-15;
more preferably, fibroblast growth factor:platelet-derived growth factor:transforming growth factor-β:glucocorticoid:heparin or its salt:vitamin C or its derivative:transferrin:insulin:progesterone:putrescine or its salt:selenite=10-30:10-30:3-20:2-5:1000-2000:10000-80000:2000-80000:100-5000:2-7:7-10000:2-7;
preferably, the mass-volume concentration of the transferrin in the bioactive substance composition is 0.1-300 μg/ml, and the mass ratio is 0.00001%-0.03%; preferably, the mass-volume concentration of the transferrin in the bioactive substance composition is 1-200 μg/ml, and the mass ratio is 0.0001%-0.02%; more preferably, the mass-volume concentration of the transferrin in the bioactive substance composition is 1-150 μg/ml, and the mass ratio is 0.0001%-0.015%;
preferably, the mass-volume concentration of the insulin in the bioactive substance composition is 0.01-50 μg/ml, and the mass ratio is 0.000001%-0.005%; preferably, the mass-volume concentration of the insulin in the bioactive substance composition is 0.1-g/ml, and the mass ratio is 0.00001%-0.003%; more preferably, the mass-volume concentration of the insulin in the bioactive substance composition is 1-20 μg/ml, and the mass ratio is 0.0001%-0.002%;
preferably, the mass-volume concentration of the progesterone in the bioactive substance composition is 0.1-50 ng/ml, and the mass ratio is 0.00000001%-0.000005%; preferably, the mass-volume concentration of the progesterone in the bioactive substance composition is 1-30 ng/ml, and the mass ratio is 0.0000001%-0.000003%; more preferably, the mass-volume concentration of the progesterone in the bioactive substance composition is 2-20 ng/ml, and the mass ratio is 0.0000002%-0.000002%.

2. The bioactive substance composition according to claim 1, wherein the fibroblast growth factor in the bioactive substance composition is selected from any one or more of FGF-basic, FGF1, FGF2, FGF4, FGF7, FGF10, FGF18, and fibroblast growth factor synthetic peptides; preferably, the mass-volume concentration of the fibroblast growth factor in the bioactive substance composition is 1-100 ng/ml, and the mass ratio is 0.0000001%-0.00001%; preferably, the mass-volume concentration of the fibroblast growth factor in the bioactive substance composition is 5-70 ng/ml, and the mass ratio is 0.0000005%-0.000007%; more preferably, the mass-volume concentration of the fibroblast growth factor in the bioactive substance composition is 10-40 ng/ml, and the mass ratio is 0.000001%-0.000004%.

3. The bioactive substance composition according to claim 1, wherein the platelet-derived growth factor in the bioactive substance composition is selected from any one or more of PDGF-AA, PDGF-AB, PDGF-BB, synthetic peptides of platelet-derived growth factor; Preferably, the mass-volume concentration of the platelet-derived factor in the bioactive substance composition is 1-100 ng/ml, accounting for 0.0000001%-0.00001% by mass; preferably, the mass-volume concentration of the platelet-derived factor in the bioactive substance composition is 5-70 ng/ml, accounting for 0.0000005%-0.000007% by mass; more preferably, the mass-volume concentration of the platelet-derived factor in the bioactive substance composition is 10-40 ng/ml, accounting for 0.000001%-0.000004% by mass.

4. The bioactive substance composition according to claim 1, wherein the transforming growth factor-β in the bioactive substance composition is selected from any one or more of TGF-β1, TGF-β2, TGF-β3, and transforming growth factor-β synthetic peptides; preferably, the mass-volume concentration of the transforming growth factor-β in the bioactive substance composition is 0.1-80 ng/ml, accounting for 0.00000001%-0.000008% by mass; preferably, the mass-volume concentration of the transforming growth factor-β in the bioactive substance composition is 2-50 ng/ml, accounting for 0.0000002%-0.000005% by mass; more preferably, the mass-volume concentration of the transforming growth factor-β in the bioactive substance composition is 5-25 ng/ml, accounting for 0.0000005%-0.0000025% by mass.

5. The bioactive substance composition according to claim 1, wherein the glucocorticoid in the bioactive substance composition is selected from any one or more of dexamethasone or its salt, dexamethasone solvent, hydrocortisone or its salt, cortisone acetate, cortisone acetate or its salt, methylprednisone sodium succinate, prednisone, betamethasone, betamethasone valerate, beclomethasone propionate, prednisolone acetate, prednisolone acetate, or prednisolone; preferably, the molar concentration of the glucocorticoid in the bioactive substance composition is 0.1-90 nM, accounting for 0.0000000039%-0.00000354% by mass; preferably, the molar concentration of the glucocorticoid in the bioactive substance composition is 1-50 nM, accounting for 0.000000039%-0.00000197% by mass; more preferably, the molar concentration of the glucocorticoid in the bioactive substance composition is 1-20 nM, accounting for 0.000000039%-0.000000785% by mass.

6. The bioactive substance composition according to claim 1, wherein the heparin or its salt in the bioactive substance composition is selected from any one or more of heparin, heparin sodium and heparin calcium; preferably, the mass volume concentration of the heparin or its salt in the bioactive substance composition is 0.1-10 μg/ml, accounting for 0.00001%-0.001% by mass; preferably, the mass volume concentration of the heparin or its salt in the bioactive substance composition is 0.5-8 μg/ml, accounting for 0.00005%-0.0008% by mass; more preferably, the mass volume concentration of the heparin or its salt in the bioactive substance composition is 1-5 μg/ml, accounting for 0.0001%-0.0005% by mass.

7. The bioactive substance composition according to claim 1, wherein the vitamin C or its derivatives in the bioactive substance composition are selected from any one or more of Vitamin C (i.e. ascorbic acid), ascorbic acid glucoside, ethyl vitamin C, 3-o-ethyl ascorbic acid, magnesium phosphate of vitamin C, sodium phosphate of vitamin C, L-ascorbic acid 2-phosphate sesquimagnesium salt complex, vitamin C tetraisopalmitate, ascorbic acid palmitate, ascorbic acid 2-phosphate 6-palmitate, esterified vitamin C, other solvates of ascorbic acid; preferably, the mass volume concentration of the vitamin C or its derivatives in the bioactive substance composition is 0.1-100 μg/ml, accounting for 0.00001%-0.01% by mass; preferably, the mass volume concentration of the vitamin C or its derivatives in the bioactive substance composition is 1-100 μg/ml, accounting for 0.0001%-0.01% by mass; more preferably, the mass volume concentration of the vitamin C or its derivatives in the bioactive substance composition is 10-80 μg/ml, accounting for 0.001%-0.008% by mass.

8. The bioactive substance composition according to claim 1, wherein the putrescine or its salt in the bioactive substance composition is selected from any one or more of putrescine and putrescine dihydrochloride; the mass volume concentration of putrescine or its salt in the bioactive substance composition is 0.01-50 μg/ml, accounting for 0.000001%-0.005% by mass; preferably, the mass volume concentration of putrescine or its salt in the bioactive substance composition is 0.1-40 μg/ml, accounting for 0.00001%-0.004% by mass; more preferably, the mass volume concentration of putrescine or its salt in the bioactive substance composition is 1-30 μg/ml, accounting for 0.0001%-0.003% by mass.

9. The bioactive substance composition according to claim 1, wherein the selenite in the bioactive substance composition is a water-soluble selenite; preferably, the selenite is sodium selenite; the mass volume concentration of the selenite in the bioactive substance composition is 0.1-50 ng/ml, accounting for 0.00000001%-0.000005% by mass; preferably, the mass volume concentration of the selenite in the bioactive substance composition is 1-30 ng/ml, accounting for 0.0000001%-0.000003% by mass; more preferably, the mass volume concentration of the selenite in the bioactive substance composition is 2-20 ng/ml, accounting for 0.0000002%-0.000002% by mass.

10. A method for preparing the bioactive substance composition, wherein the preparation of the bioactive substance composition comprises the following steps: mixing fibroblast growth factor, platelet-derived growth factor, transforming growth factor-β, glucocorticoid, heparin or its salt, vitamin C or its derivatives, transferrin, insulin, progesterone, putrescine or its salt and selenite in proportion; the addition order of each component is not in order; the mass volume concentration range of each component is:
fibroblast growth factor:platelet-derived growth factor:transforming growth factor-β:glucocorticoid:heparin or its salt:vitamin C or its derivative:transferrin:insulin:progesterone:putrescine or its salt:selenite=1-50:1-50:1-40:1-11:10-5000:10-100000:10-300000:1-25000:1-25:1-25000:1-25;
preferably, fibroblast growth factor:platelet-derived growth factor:transforming growth factor-β:glucocorticoid:heparin or its salt:vitamin C or its derivative:transferrin:insulin:progesterone:putrescine or its salt:selenite=5-40:5-40:2-30:1-8:500-4000:1000-90000:1000-200000:10-15000:1-15:2-15000:1-15;
more preferably, fibroblast growth factor:platelet-derived growth factor:transforming growth factor-β:glucocorticoid:heparin or its salt:vitamin C or its derivative:transferrin:insulin:progesterone:putrescine or its salt:selenite=10-30:10-30:3-20:2-5:1000-2000:10000-80000:2000-80000:100-5000:2-7:7-10000:2-7;
preferably, the mass-volume concentration of the transferrin in the bioactive substance composition is 0.1-300 μg/ml, and the mass ratio is 0.00001%-0.03%; preferably, the mass-volume concentration of the transferrin in the bioactive substance composition is 1-200 μg/ml, and the mass ratio is 0.0001%-0.02%; more preferably, the mass-volume concentration of the transferrin in the bioactive substance composition is 1-150 μg/ml, and the mass ratio is 0.0001%-0.015%;
preferably, the mass-volume concentration of the insulin in the bioactive substance composition is 0.01-50 μg/ml, and the mass ratio is 0.000001%-0.005%; preferably, the mass-volume concentration of the insulin in the bioactive substance composition is 0.1-30 μg/ml, and the mass ratio is 0.00001%-0.003%; more preferably, the mass-volume concentration of the insulin in the bioactive substance composition is 1-20 μg/ml, and the mass ratio is 0.0001%-0.002%;
preferably, the mass-volume concentration of the progesterone in the bioactive substance composition is 0.1-50 ng/ml, and the mass ratio is 0.00000001%-0.000005%; preferably, the mass-volume concentration of the progesterone in the bioactive substance composition is 1-30 ng/ml, and the mass ratio is 0.0000001%-0.000003%; more preferably, the mass-volume concentration of the progesterone in the bioactive substance composition is 2-20 ng/ml, and the mass ratio is 0.0000002%-0.000002%;
preferably, the temperature for the mixing is 0-37° C.

11. A serum-free medium, wherein the serum-free medium comprises a basic culture medium and additional components comprising a bioactive substance composition described in claim 1; the serum-free medium is a complete serum-free medium; preferably, the culture refers to primary culture and secondary culture of cells and/or tissues; more preferably, the culture refers to maintaining the proliferation and phenotype of cells and/or tissues, or enhancing the proliferation and phenotype of cells and/or tissues;
preferably, the cell common characteristics and cell-specific phenotypes of the cells cultured in the serum-free medium all reach their respective pass lines, and the total cell score reaches more than 60 points.

12. The serum-free medium according to claim 11, wherein the basic medium is selected from any one or more of DMEM low sugar medium, DMEM high sugar medium, DMEM/F12 medium, F12 medium, F10 medium, MEM medium, BEM medium, RPMI 1640 medium, Media 199 medium, IMDM medium, mTesR medium and E8 medium.

13. A composition, wherein the composition comprises at least one bioactive component and at least one additive, and the bioactive component is selected from any of the bioactive substance compositions of claim 1;
preferably, the cell common characteristics and cell-specific phenotypes of the cells cultured by the composition all reach their respective pass lines, and the total cell score reaches more than 60 points; preferably, the cell common characteristics and cell-specific phenotypes of the cells cultured by the composition all reach their respective pass lines, and the total cell score reaches more than 80 points; preferably, the cell common characteristics and cell-specific phenotypes of the cells cultured by the composition all reach their respective pass lines, and the total cell score reaches more than 90 points; more preferably, the cell common characteristics and cell-specific phenotypes of the cells cultured by the composition all reach their respective pass lines, and the total cell score reaches more than 100 points.

14. The composition according to claim 13, wherein the additive is selected from any one or more of cell culture additives, growth factors, small molecule drugs, hormones, vitamins, wall promoting substances, macromolecular proteins, synthetic peptides, amino acids, lipids, enzymes, carbohydrate, pH regulating substances, trace elements and antibiotics;
the cell culture additive comprises one or more of B27 cell culture additive, N2 cell culture additive, chemically defined lipid concentrate, ITS, and fatty acid additive; More preferably, calculated by the total volume of the composition, the concentration of the cell culture additive in the composition is 0.1-5×; more preferably, based on the total volume of the composition, the concentration of the cell culture additive in the composition is 0.5-2×;
the growth factor comprises one or more of vascular endothelial growth factor, vascular endothelial growth factor synthetic peptide, epidermal growth factor, epidermal growth factor synthetic peptide, insulin-like growth factor, insulin-like growth factor synthetic peptide, nerve growth factor, nerve growth factor synthetic peptide, colony stimulating factor, colony stimulating factor synthetic peptide, growth hormone release inhibiting factor, growth hormone release inhibiting factor synthetic peptide; the mass volume concentration of the growth factor is 1-100 ng/ml; preferably, the mass volume concentration of the growth factor is 1-50 ng/ml; more preferably, the mass volume concentration of the growth factor is 5-40 ng/ml;
preferably, the small molecule drug is selected from GSK3 inhibitor; the GSK3 inhibitor is selected from CHIR99021; preferably, the molar concentration of the small molecule drug is 0.1-10 μM; more preferably, the molar concentration of the small molecule drug is 0.1-5 μM;
preferably, the amino acid is selected from any one or more of nonessential amino acids, L-glutamic acid and L-glutamine; more preferably, the molar concentration of the amino acid is 0.01-4 mM;
preferably, the carbohydrate is sodium pyruvate; more preferably, the mass volume concentration of the carbohydrate is 0.01-2 mM;
preferably, the pH maintaining agent is selected from any one or more of 4-hydroxyethyl piperazine ethanesulfonic acid (HEPES) and L-glyceryl phosphate disodium salt water complexes; more preferably, the molar concentration of the pH maintaining agent is 1-20 mM;
preferably, the adhesion promoting substance is selected from any one or more of laminin, fibronectin, vitronectin, collagen, gelatin and the synthetic peptide of the adhesion promoting substance; preferably, the mass volume concentration range of laminin is 0.1-100 μg/ml; preferably, the mass volume concentration range of fibronectin is 0.1-200 μg/ml; preferably, the mass volume concentration range of vitronectin is 0.1-100 μg/ml; preferably, the mass volume concentration range of collagen is 0.1-100 μg/ml; preferably, the mass volume concentration range of gelatin is 0.1-100 μg/ml; preferably, the mass volume concentration range of synthetic peptides of the adhesion promoting substance is 0.1-100 μg/ml;
preferably, the antibiotic is selected from any one or more of penicillin, streptomycin and gentamicin; more preferably, the mass volume concentration range of the antibiotic is 50-100 μg/mL.

15. A usage of the bioactive substance composition of claim 1, wherein the use is selected from the culture of cells and/or tissues, or the use in the preparation of tissue and/or organ injury treatment drugs;
preferably, the cells are selected from any one or more of tendon and/or ligament derived cells, mesenchymal stem cells, meniscal stem cells, chondrocytes, skeletal stem cells, and muscle stem cells;
preferably, the tissue is the tissue derived from the musculoskeletal system; preferably, the tissue derived from the musculoskeletal system is selected from tendon tissue, ligament tissue, meniscus tissue, cartilage tissue, fat tissue and muscle tissue;
preferably, the tissue and/or organ injury is the tissue and/or organ injury of the musculoskeletal system; preferably, the tissue and/or organ injury of the musculoskeletal system is selected from at least one of tendon and/or ligament injury, cartilage injury, bone injury, muscle injury, skin injury, and blood vessel injury.

16. A cell or tissue culture method, wherein the culture method comprises the step of contacting cells and/or tissues with a serum-free medium and/or a composition; the serum-free medium is the serum-free medium as described in claim 11; preferably, the culture method is selected from suspension culture method and adherent culture method; preferably, the adherent culture method is selected from the method of coating the surface of culture carriers by adhesion promoting substance, and the method of adding the adhesion promoting substance to the culture medium;
preferably, the method of coating the surface of culture carriers by adhesion promoting substance comprises the following steps: 1) Treating the culture carrier with the adhesion promoting substance, preferably, the culture carrier is selected from at least one of the pore plate, culture dish, culture bottle, microcarrier, microsphere, microarray and bioactive material; 2) Inoculate cells and/or tissues into the culture carriers treated in step 1); 3) Add to the serum-free medium and/or the composition for culture;
more preferably, the method of adding the adhesion promoting substance to the culture medium comprises the following steps: 1) inoculating cells and/or tissues into a culture carrier, preferably, the culture carrier is selected from at least one of the pore plates, culture dishes, culture bottles, microcarriers, microspheres, microarrays, and bioactive materials; 2) Add the adhesion promoting substance directly to the serum-free medium and/or the composition, and then add it to the culture carrier in step 1) for cell culture;
preferably, the suspension culture method comprises the following steps: 1) Inoculate cells and/or tissues into low-adhesive or non-adhesive culture well plates, culture dishes, culture flasks, other culture carriers, cell dynamic culture bioreactors; 2) Add the serum-free medium and/or composition for culture;
preferably, the cells are selected from any one or more of tendon and/or ligament derived cells, mesenchymal stem cells, meniscal stem cells, chondrocytes, skeletal stem cells, and muscle stem cells;
preferably, the tissue is the tissue derived from the musculoskeletal system; preferably, the tissue derived from the musculoskeletal system is selected from tendon tissue, ligament tissue, meniscus tissue, cartilage tissue, fat tissue and muscle tissue;
preferably, the adhesion promoting substance is selected from any one or more of laminin, fibronectin, vitronectin, collagen, gelatin and the synthetic peptide of the adhesion promoting substance;
preferably, the synthetic peptide of the adhesion promoting substance is a synthetic polypeptide, oligopeptide or amino acid sequence that can replace the adhesion promoting substance to promote cell adhesion, including any one or more of laminin synthetic peptide, fibronectin synthetic peptide, fibronectin synthetic peptide, RGD (Arg Gly Asp) peptide, KRSR (Lys Arg Ser Arg) peptide;
preferably, the concentration range of laminin is 0.1-100 μg/ml, and/or the concentration range of fibronectin is 0.1-200 μg/ml, and/or fibronectin 0.1-100 μg/ml, and/or the concentration range of collagen is 0.1-100 mg/ml, and/or the concentration of gelatin is 0.1-100 mg/ml;
preferably, the concentration range of laminin synthetic peptide is 0.1-100 μg/ml, and/or the concentration range of the fibronectin synthetic peptide is 0.1-200 μg/ml, and/or hyaluronan synthetic peptide 0.1-100 μg/ml, and/or the RGD (Arg-Gly-Asp) peptide concentration range is 50-1000 mg/ml, and/or the KRSR (Lys-Arg-Ser-Arg) peptide concentration range is 50-1000 mg/ml.

17. A cell and/or tissue, wherein the cell and/or tissue are obtained by culturing in the serum-free medium and/or the composition; the serum-free medium is prepared by the method of claim 11;
preferably, the cells are selected from any one or more of tendon and/or ligament derived cells, mesenchymal stem cells, meniscal stem cells, chondrocytes, bone stem cells, and muscle stem cells;
preferably, the tissue is the tissue derived from the musculoskeletal system; preferably, the tissue derived from the musculoskeletal system is selected from tendon tissue, ligament tissue, meniscus tissue, cartilage tissue, fat tissue and muscle tissue;
preferably, the scores of each single item in the cell common features and cell-specific phenotypes of the cells reach their respective pass lines, and the total score of the cells reaches more than 60 points; preferably, the scores of each single item in the cell common features and cell-specific phenotypes of the cells reach their respective pass lines, and the total score of the cells reaches more than 80 points; preferably, the scores of each single item in the cell common features and cell-specific phenotypes of the cells reach their respective pass lines, and the total score of the cells reaches more than 90 points; more preferably, the scores of each single item in the cell common features and cell-specific phenotypes of the cells reach their respective pass lines, and the total score of the cells reaches more than 100 points.
